(19) United States
(12) Patent Application Publication
(10) Pub. No.: US 2024/0002874 A1
(43) Pub. Date: Jan. 4, 2024

(54) INCREASING PHOTOSYNTHETIC CAPACITY IN PLANTS

(71) Applicant: University of Essex Enterprises Limited, Colchester (GB)

(72) Inventors: Philip M Mullineaux, Colchester (GB); Ellie J. Stallard, Colchester (GB)

(73) Assignee: University of Essex Enterprises Limited, Colchester (GB)

(21) Appl. No.: 18/257,836

(22) PCT Filed: Nov. 18, 2021
(86) PCT No.: PCT/GB2021/052987

§ 371 Date: Jun. 15, 2023

(30) Foreign Application Priority Data
Dec. 18, 2020 (GB) 2020158.8

(51) Int. Cl. C12N 15/82; C12Q 1/6895

(52) U.S. Cl. CPC C12N 15/8269; C12Q 1/6895; C12Q 2600/156

(57) ABSTRACT

The invention relates to methods of increasing photosynthetic capacity in plants by modulating the cryptochrome 1 (CRY1)-directed signalling pathway. In certain embodiments, the invention relates to removing constraints on maximal photosynthetic capacity by reducing or abolishing SPA gene expression and/or activity. The invention further relates to plants having increased photosynthetic capacity, identifying plants with increased photosynthetic capacity and methods of producing food or feed products.

### FIELD OF THE INVENTION

The present invention relates to methods of increasing photosynthetic capacity in plants by modulating the cryptochrome 1 (CRY1)-directed signalling pathway. In certain embodiments, the invention relates to removing constraints on maximal photosynthetic capacity by reducing or abolishing SPA gene expression and/or activity. The invention further relates to plants having increased photosynthetic capacity, identifying plants with increased photosynthetic capacity and methods of producing food or feed products.

### BACKGROUND TO THE INVENTION

There is an urgent need to increase productivity of crop species to keep pace with a world population that will peak in 2050. Yield increases delivered by conventional plant breeding has slowed or ceased for all major commodity crops. Therefore, there is an urgent need to provide new solutions for enhancing crop productivity. Whereas modern varieties of crops have attained near theoretical maxima for light energy interception by leaf canopies and the partitioning of photosynthate to the harvestable crop product (e.g. seed), conventional breeding has made virtually no gains on the conversion efficiency of light energy to chemical energy and photosynthate.

The exposure of plants to increased light intensities can lead to the development of enhanced photosynthetic capacity in mature leaves and is termed dynamic acclimation. Dynamic acclimation is an important determinant of plant fitness (or crop yield), is under genetic as well as environmental control and includes changes in the expression of many genes (Murchie and Horton, 1997; Walters et al., 1999; Oguchi et al., 2003; Murchie et al., 2005; Eberhard et al., 2008; Athanasiou et al., 2010; Schottler and Toth, 2014; van Rooijen et al 2015; Vialet-Chabrand et al., 2017).

In young expanding leaves, acclimation to increased light intensity (i.e., high light) brings about increased photosynthetic capacity by eliciting changes in both leaf morphology and changes to chloroplast physiology. In contrast, in mature leaves exposure to sustained or episodic high light brings about changes primarily in chloroplast physiology that raise the light use efficiency of photosynthesis, which can reflect increased rates of photosynthesis and/or a decreased number of photosystem II (PSII) reaction centers (Walters et al., 1999; Murchie et al., 2005; Athanasiou et al., 2010; van Rooijen et al., 2015; Vialet-Chabrand et al., 2017).

How high light exposure initiates chloroplast-level dynamic acclimation remains poorly understood. Current approaches to increasing photosynthetic capacity are to manipulate the ability of plants to dissipate excess excitation energy, engineer bypasses or shunts in the photorespiratory cycle and liner electron flux or by over-expressing components of primary metabolism and the Calvin-Benson cycle. However, such approaches require re-engineering of the primary metabolism or photosynthetic apparatus of the plant. This may lead to adverse pleiotropic effects, and the impact of such interventions on the plant's ability to deal with environmental stress remains unknown.

There remains a need for methods of increasing photosynthetic capacity in plants. It is an aim of certain embodiments of the present invention to at least partly mitigate the above-mentioned problems associated with the prior art.

### SUMMARY OF CERTAIN EMBODIMENTS OF THE INVENTION

The invention relates to the finding that manipulating the cryptochrome 1 (CRY1)-directed signalling pathway promotes the maximum photosynthetic capacity that mature fully expanded leaves (so-called photosynthate source leaves that drive plant productivity) can achieve when challenged with an increased light intensity.

In certain embodiments, the invention relates to the unexpected finding that Suppressor of PhyA-105 (SPA) proteins are negative regulators of photosynthetic capacity. Moreover, the inventors have shown that Constitutively Photomorphogenic 1 (COP1) proteins, that interact with SPA proteins, also constrain maximum photosynthetic capacity in plants.

The inventors' have also shown that B-BOX DOMAIN CONTAINING PROTEIN32 (BBX32) is a negative regulator of photosynthetic capacity and works in tandem with a second gene, LONG HYPOCOTYL5 (HY5), which is a positive regulator of this process. Furthermore, elucidation of the gene regulatory networks controlling dynamic acclimation to high light also reveals that Cryptochrome 1 (CRY1) and Phytochrome B (PHYB) are positive regulators of photosynthetic capacity.

In summary, elucidation of the CRY1-directed signalling pathways herein allows the development of crops with improved fitness and/or yield. The invention shows that it is possible to release negative controls that cause genes such as SPA to constrain photosynthetic capacity and thereby achieve a substantial increase in photosynthesis and therefore yield. Furthermore, this is achieved by exploiting plants' intrinsic photosynthetic capacity and not be carrying out re-engineering of primary metabolism or the photosynthetic apparatus.

Accordingly, the invention provides a method of increasing photosynthetic capacity in a plant, the method comprising modulating Cryptochrome 1 (CRY1)-directed signalling in the plant. Typically, the method comprises the step of determining photosynthetic capacity. For example, the method may comprise quantifying photosystem II (PSII) operating efficiency, linear electron flux and/or quantum yield of CO₂ assimilation in the plant (e.g., in mature leaves of the plant) as further described herein.

In certain embodiments, the invention provides any method of increasing photosynthetic capacity in a plant (e.g., in mature leaves of the plant) as described herein, wherein the plant is determined to have increased photosynthetic capacity as compared to a control plant (e.g., a plant not having modulated CRY1-directed signalling.)

In preferred embodiments, modulating CRY1-directed signalling comprises reducing or abolishing the expression of at least one nucleic acid sequence encoding a SPA polypeptide (e.g. one, two or three of SPA1, SPA2, SPA3 and/or SPA4) and/or reducing or abolishing the activity of a SPA polypeptide in the plant.

Any suitable method of reducing or abolishing expression and/or activity of SPA may be used, including, but not limited to, mutagenic procedures resulting in SPA genes in a target species being deleted or inactivated (e.g. insertional mutagenesis using transposons or T-DNA, chemical mutagenesis, gene editing, antisense or interfering RNA procedures, using constitutive or inducible promoters as further described herein).

CRY1-directed signalling has ancient evolutionary origins, and SPA genes are present and well conserved across the Streptophyte lineage, meaning that all land plants have these genes. As such, the method of the invention is applicable to any suitable crop species.

The invention also provides plants (e.g. crop plants such as tomato, barley etc.) having modulated CRY-directed signalling, wherein the PSII operating efficiency (Fq′/Fm′) in the plant is increased by at least about 10%, 20%, 30%, 40%, 50%, 60%, 70%, 80%, 90%, 100% or more as compared to a control or wild-type plant (typically wherein the actinic photosynthetically active photon flux density (PFFD) is about 800 μmol m⁻² s⁻¹). Preferably, the PSII operating efficiency is increased by at least about 40% or more as compared to a control or wild-type plant, wherein the actinic PFFD is about 800 μmol m⁻² s⁻¹.

The invention also provides methods of identifying one or more alleles associated with increased photosynthetic capacity in one or more plants, the method comprising:
(a) detecting in the plant(s) one or more polymorphism(s) in a nucleic acid sequence encoding any polypeptide described herein (e.g. SPA polypeptide), wherein the one or more polymorphism(s) are associated with increased photosynthetic capacity; and
(b) identifying one or more allele(s) at the one or more polymorphism(s) that are associated with increased photosynthetic capacity.

The invention also provides methods of producing a food or feed product in a plant grown under controlled light conditions, the method comprising:
(a) obtaining a plant having increased photosynthetic capacity according to any method described herein;
(b) isolating a plant part or seed from the plant; and
(c) producing a food or feed product from the plant part or seed.

The invention also provides a method of quantifying photosynthetic capacity in one or more plants having modulated CRY-directed signalling, wherein the method comprises:
(a) subjecting the plant to any high light (HL) conditions as described herein;
(b) subjecting the plant to any low light (LL) conditions as described herein;
(c) subjecting the plants to increasing actinic PPFD as described herein; and
(d) quantifying PSII operating efficiency, linear electron flux and/or quantum yield of CO₂ assimilation in the one or more plants.

### DETAILED DESCRIPTION OF EMBODIMENTS OF THE INVENTION

Embodiments of the present invention will now be described hereinafter, by way of example only, with reference to the accompanying drawings in which:

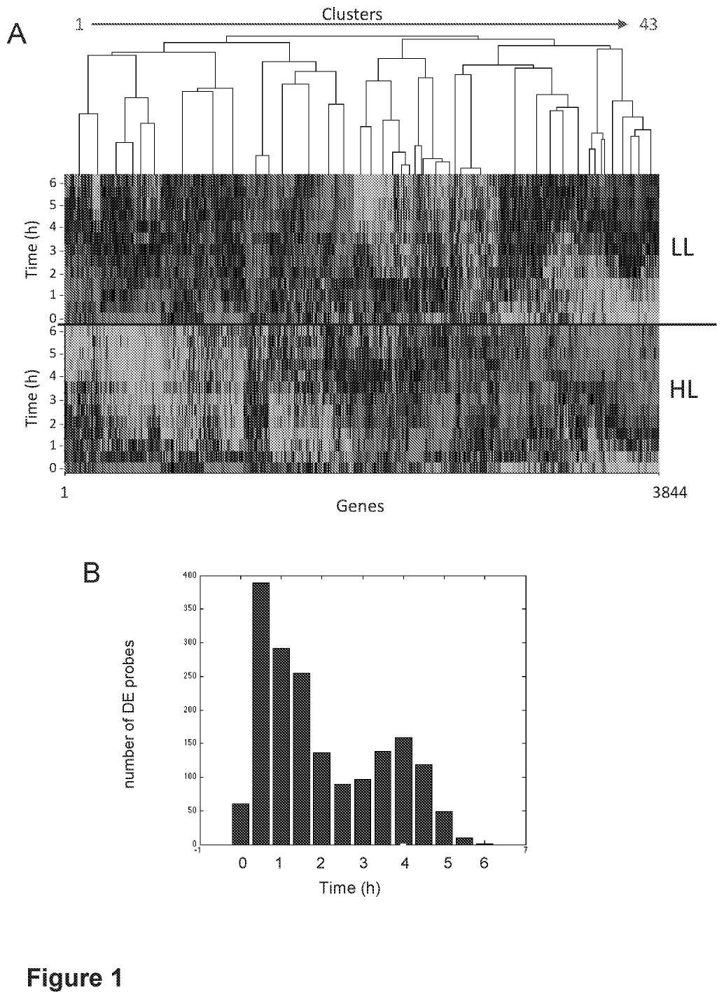

FIG. 1 shows temporal patterns of gene expression in low light (LL)- and high light (HL)-exposed leaf 7. (A) Visual output of co-clustered expression values by SplineCluster. This was done for the 3844 genes already identified as differentially expressed in HL vs LL over the time of the experiment (see Results). The values range from log₂ 2.5 (red) to −log₂ 2.0 (green). The 43 temporal clusters can be counted in the accompanying dendrogram. The timepoints are shown on the y-axes for the HL and LL gene expression. (B) The number of HL/LL differentially expressed probes at each time point.

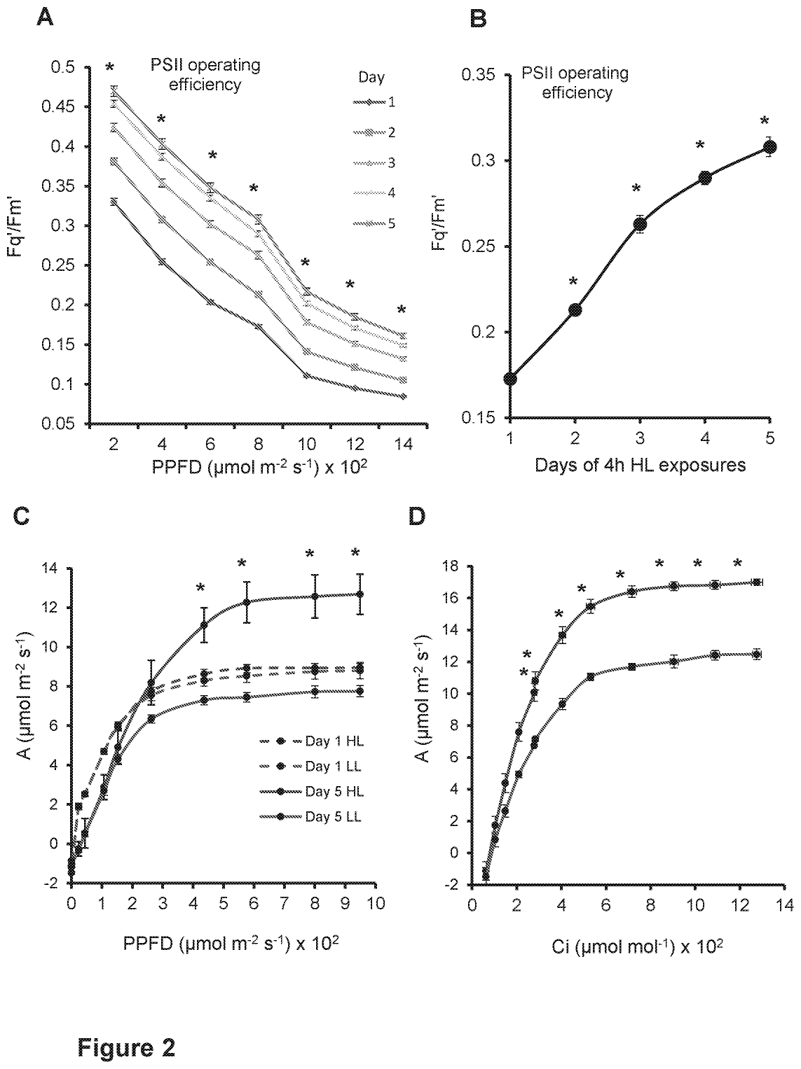

FIG. 2 shows induction of dynamic acclimation by repeated daily exposure to HL. (A) Plants were exposed daily to 4 h HL and Fq′/Fm′ determined for mature leaves. After the HL, plants were dark adapted and imaged under increasing actinic PPFD from 200 to 1400 μmol m⁻² s⁻¹ in 200 μmol m⁻² s⁻¹ increments every 5 min. The data were collected as CF images and processed digitally to collect values from mature leaves. The plants were treated in this way daily for 5 days: day 1 (blue), day 2 (red), day 3 (olive green), day 4 (purple) and day 5 (light blue). The data (mean±SE) correspond to 38 plants at 24-28 dpg over 6 experiments and the asterisks show differences in CF parameters between days 1 and 5 were significant (P ANOVA and TukeyHSD). (B) Daily changes in Fq′/Fm′ plotted from the data in A and supplementary data (right panel). Fq′/Fm′ values are from the same plants over the daily HL exposures showing the increase in PSII operating efficiency at 800 μmol m⁻² s⁻¹ PPFD actinic light over the 5 days of the experiments. (C) Photosynthesis plotted as CO₂ assimilation rate (A) as a function of actinic PPFD in mature leaf 7 (mean±SE; n=8 plants for each treatment; 49 dpg). Measurements were taken the day after 1 (dashed lines) and 5 days (solid lines) of daily 4 h HL exposures (blue lines) along with the LL control plants (red lines) not subjected to this treatment. (D) Photosynthesis plotted as CO₂ assimilation rate (A) as function of leaf internal CO₂ concentration (Ci) in mature leaf 7 (mean±SE; n=8 plants for each treatment; 49 dpg). Measurements were taken the day after 5 days of daily 4 h HL exposures (blue line) along with the LL control (red line). A was determined by Infra-Red Gas Analysis (see Methods). Asterisks indicate significant differences (P<0.02; covariant T and two-tailed F tests) between LL and HL-exposed plants.

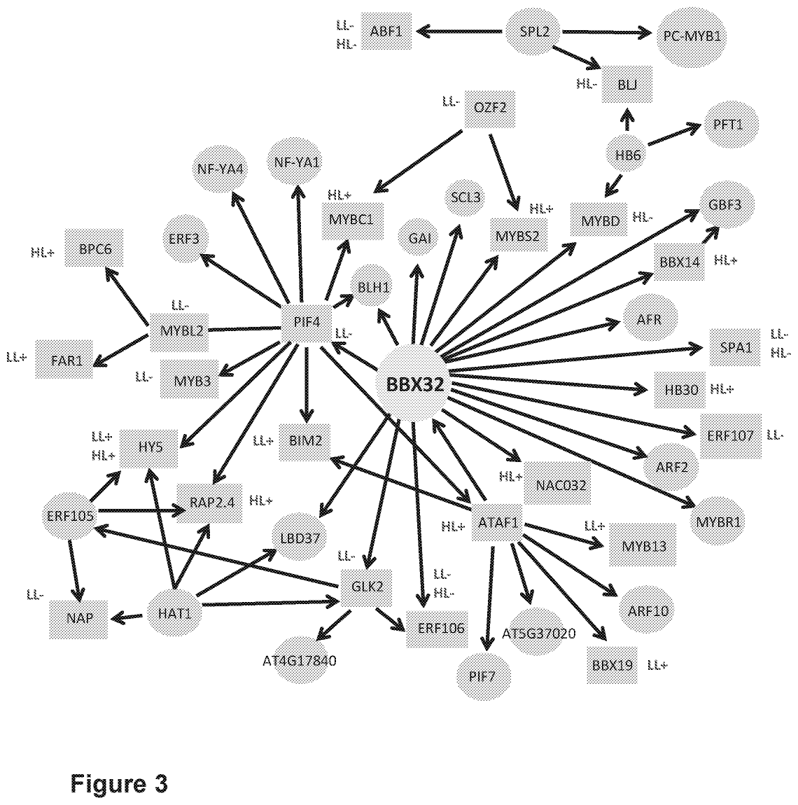

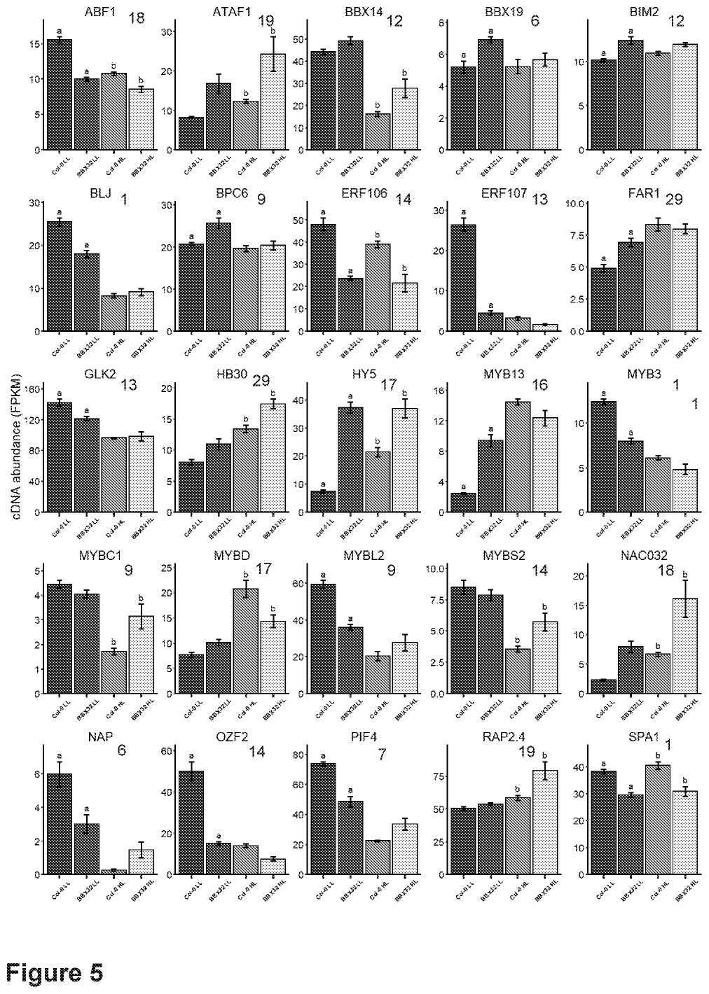

FIG. 3 shows inferred HL gene regulatory network. The network shown was generated from the time series expression data for HL DEGs. The DEGs code for transcription (co)factors that are also light and/or PHYA/PHYB regulated in de-etiolating seedlings. The network was generated using Variational Bayesian State Space Modelling (VBSSM; threshold z-score=2.33; see Methods) and initially visualised using Cytoscape (v3.3.2; Shannon et al., 2003) but re-drawn manually to improve clarity. The network shown is from the second iteration of the modelling, which omitted expression data for LHY. The genes depicted in rectangular nodes were responsive to BBX32 over-expression in HL and/or LL exposed leaves and showed significantly (P<0.05; Tukey HSD) by showing higher (+) or lower (−) transcript abundance than Col-0 (see FIG. 5). Locus codes for the network genes can be found in Methods.

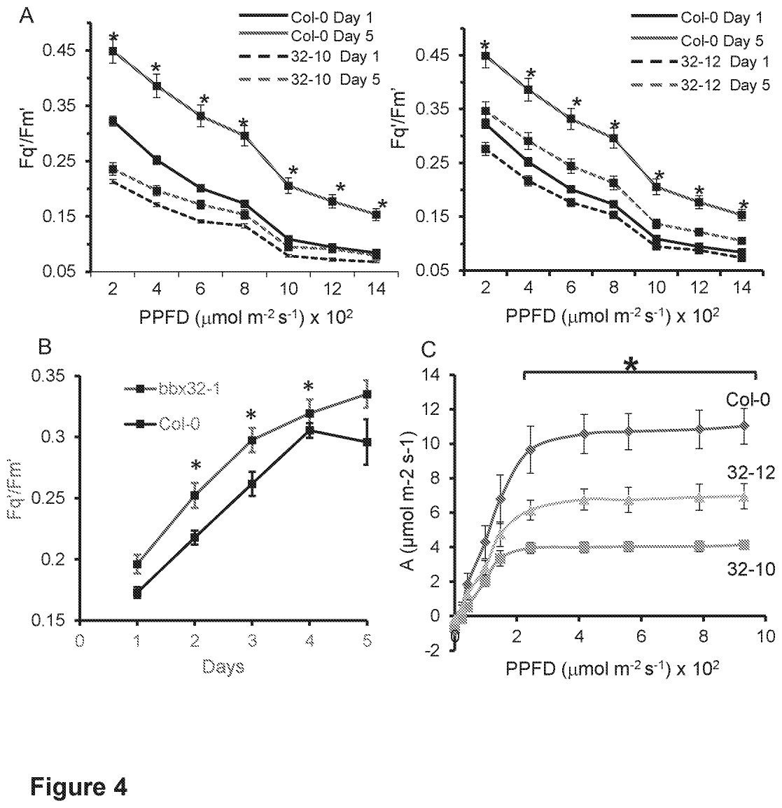

FIG. 4 shows dynamic acclimation in BBX32-OE and bbx32-1 plants. Fq′/Fm′ values determined from images of 4 mature leaves from 8 plants (24-28 dpg) over 2 experiments (means±SE) which had first been exposed to 4 h HL each day for 5 consecutive days (see Methods and legend of FIG. 2). CF parameter values were collected at a range of actinic PPFDs (as indicated) at the end of each daily HL exposure. (A) Fq′/Fm′ values at day 1 (black lines) and day 5 (red lines) for mutant or OE plants (dashed line) and Col-0 (solid line) of the HL treatments for BBX32-10 and BBX32-12. Asterisks indicate difference between mutant genotype and Col-0 at day 5 (P<0.01; ANOVA and TukeyHSD). (B) Daily Fq′/Fm′ values at 800 μmol m-2 s-1 PPFD actinic light of bbx32-1 compared with Col-0 showing differences that were significant (P<0.01) only between days 2 and 4. (C) Photosynthesis plotted as CO₂ assimilation rate (A) as a function of incident PPFD in mature leaf 7 of LL-grown BBX32-10 (green line) and BBX32-12 (red line) compared to Col-0 (blue line) plants. Data are the mean±SE; n=4 for each genotype at 49 dpg; Asterisk indicates significant differences (P<0.02; covariant T and two-tailed F tests) between Col-0 and BBX32-10 and BBX32-12 at a given PPFD. Leaf A, as a function of PPFD, was determined by Infra-Red Gas Analysis (see Methods).

FIG. 5 shows partial validation of the BBX32-centric inferred gene regulatory network. The expression of 25 of the 47 TF genes in the inferred network showing the effect of BBX32 over-expression. All the genes displayed significant differences (p<0.05; ANOVA and Tukey HSD) in cDNA abundance in BBX32-OE plants compared with Col-0 under LL (suffix “a”) and/or HL (suffix “b”) conditions. Color codes are dark red and blue Col-0 and BBX32-OE plants in LL respectively, salmon pink and light blue are Col-0 and BBX32-OE plants in HL. The cluster number for each gene is shown on each graph.

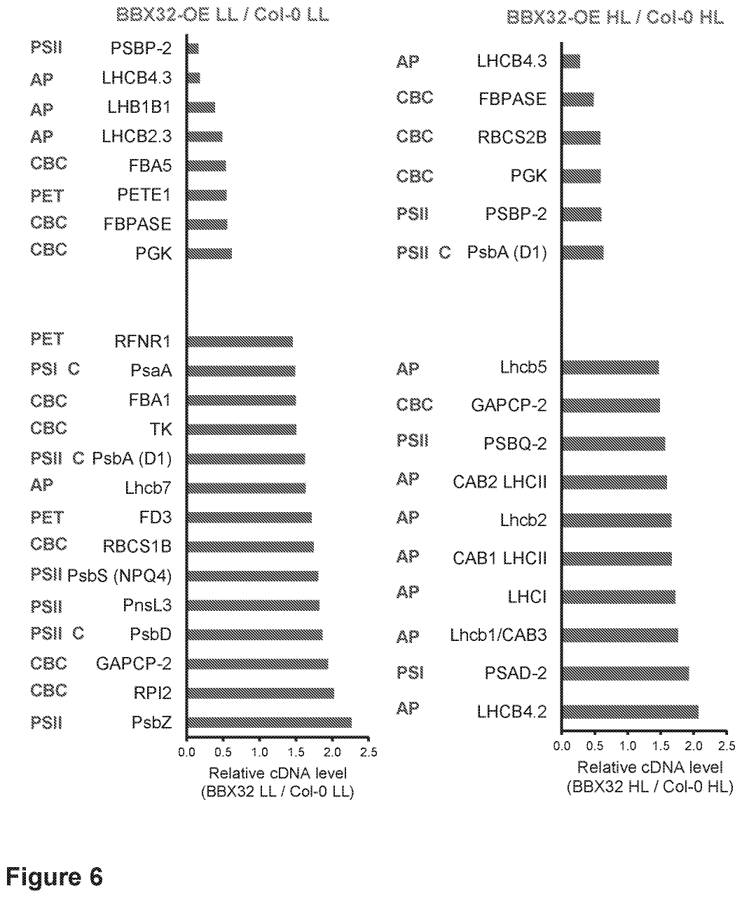

FIG. 6 shows BBX32 over-expression in LL and HL-exposed leaves perturbs transcript level of photosynthesis-associated genes. Using RNAseq data, relative cDNA abundance of BBX32-OE compared with Col-0 of photosynthesis associated genes was determined under LL and 3.5 h HL exposure. The transcripts encoding the above proteins all displayed a >1.45-fold greater or lesser abundance in fully expanded leaves of BBX32-OE plants. The values are calculated from mean FPKM values (n=4) and difference between Col-0 LL and Col-0 HL were significant for photosynthesis associated genes (https://www.kegg.jp/dbget-bin/www_bget?pathway+ath00195): AP, Antenna Protein; CBC, Calvin-Benson cycle enzyme; PET, photosynthetic electron transport protein; PSI and PSII, Photosystem I and II component proteins respectively. Most proteins are nuclear encoded but those marked with the suffix “C” are plastid encoded. Locus codes for the genes can be found in Methods.

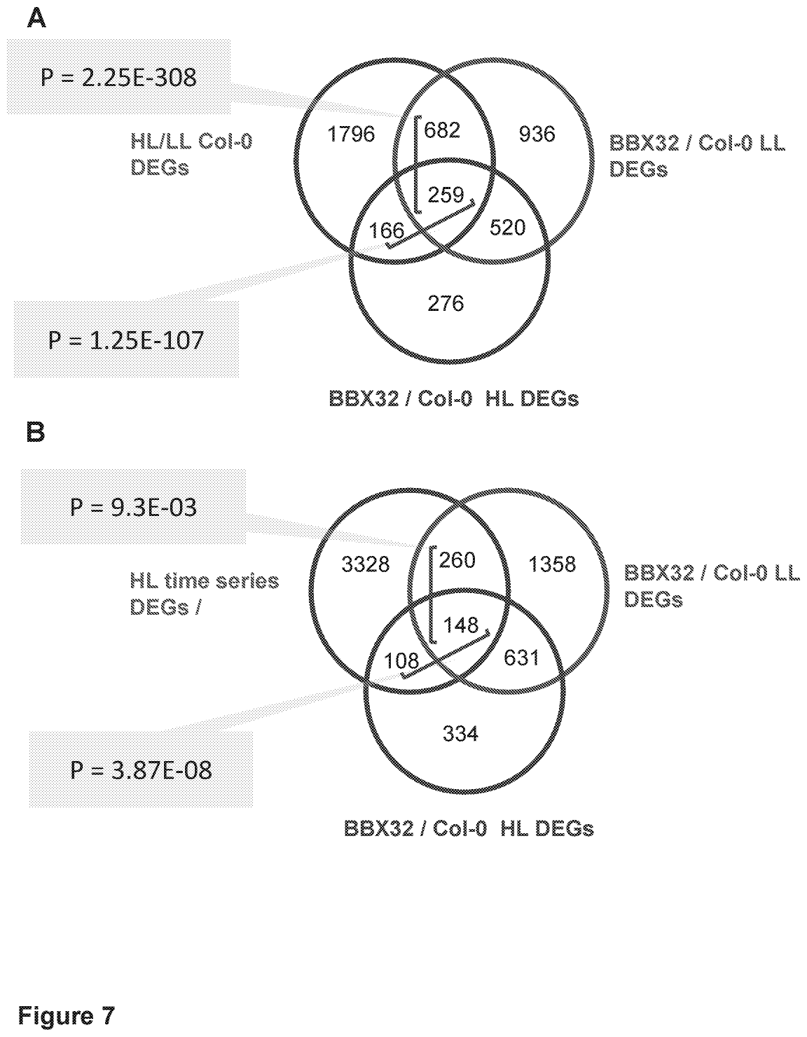

FIG. 7 shows comparisons of genes affected by BBX32 over-expression with DEGs responsive to HL in Col-0. (A) Venn diagram of overlapping DEGs between Col-0 and BBX32-OE plants under LL and HL conditions compared with DEGs responsive in Col-0 to 3.5 h HL and generated by RNAseq. (B) Venn diagram as in (A) except the BBX32-OE DEGs were compared with the time series HL DEGs, which were derived from microarray-based transcriptomics data (see Results and Methods).

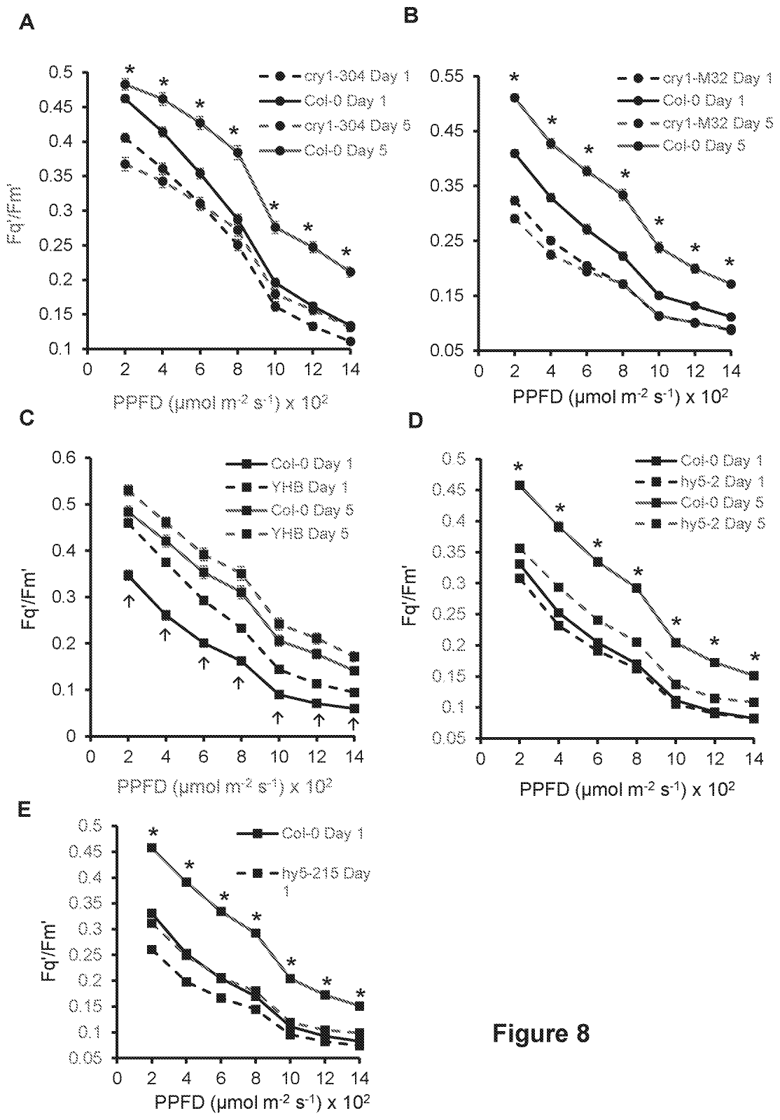

FIG. 8 shows dynamic acclimation of photoreceptor and HY5 mutants. The plots show the PSII operating efficiencies (Fq′/Fm′) determined from CF images of 4 mature leaves from 8 plants (24-28 dpg) over 2 experiments (means±SE). The plants had been exposed to 4 h HL each day for 5 consecutive days (see Methods and legend of FIG. 2). CF parameter values were collected at a range of actinic PPFDs (as indicated on the x-axis) at the end of days 1 and 5 of HL. The Fq′/Fm′ values at day 1 (black lines) and day 5 (red lines) for mutant plants (dashed line) and Col-0 (solid line) of the HL treatments for (A) cry1-304, (B) cry1-M32, (C) YHB, (D) hy5-2 and (E) hy5-215. Asterisks (panels A, B, D, E) indicate significant difference between mutant compared with Col-0 at day 5 (P<0.01; ANOVA and TukeyHSD). Upward arrows (panel C) indicate significant difference between YHB and Col-0 at day 1 (P<0.01; ANOVA and TukeyHSD).

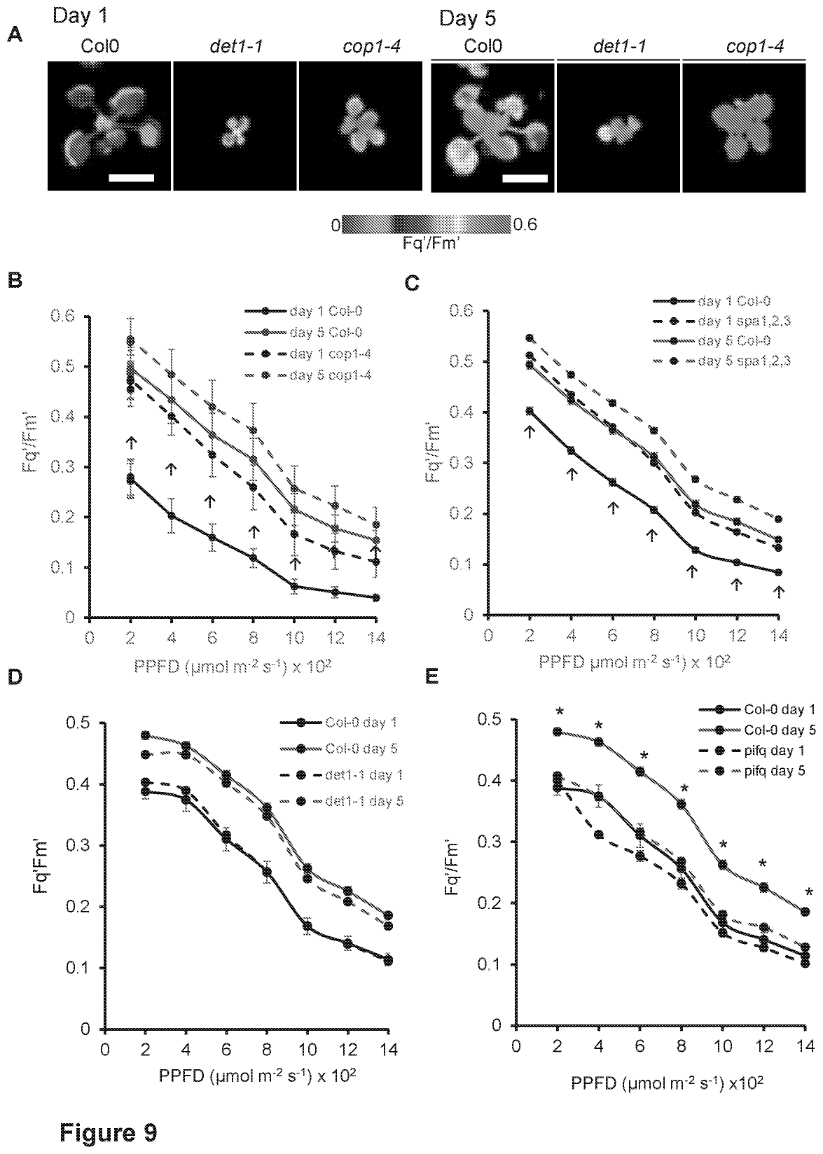

FIG. 9. Dynamic acclimation of photoreceptor signal transduction mutants. (A) Photosynthetic efficiency of the same single representative Col-0, cop1-4 and det1-1 plants after 1 and 5 days of daily 4 h HL exposure. The CF images are of Fq′/Fm′ (PSII operating efficiency) at a 400 μmol m⁻² s⁻¹ actinic PPFD. (B-E) The plots show the PSII operating efficiencies (Fq′/Fm′) determined from CF images of from 8 plants (24-28 dpg) over 2 experiments (means±SE). The plants had been exposed to 4 h HL each day for 5 consecutive days (see Methods and legend of FIG. 2). Note that because of the size of the cop1-4, pifq and det1-1 plants, data were collected from whole rosettes rather than from mature leaves. CF parameter values were collected at a range of actinic PPFDs (as indicated on the x axis) at the end of days 1 and 5 of HL. The Fq′/Fm′ values at day 1 (black lines) and day 5 (red lines) for mutant plants (dashed line) and Col-0 (solid line) of the HL treatments for (B) cop1-4, (C) spa1,2,3, (D) det1-1 and (E) pifQ. Asterisks (panel E) indicate significant difference between mutant compared with Col-0 at day 5 (P<0.01; ANOVA and TukeyHSD). Upward arrows (panels B, C) indicate significant difference between mutants and Col-0 at day 1 (P<ANOVA and TukeyHSD).

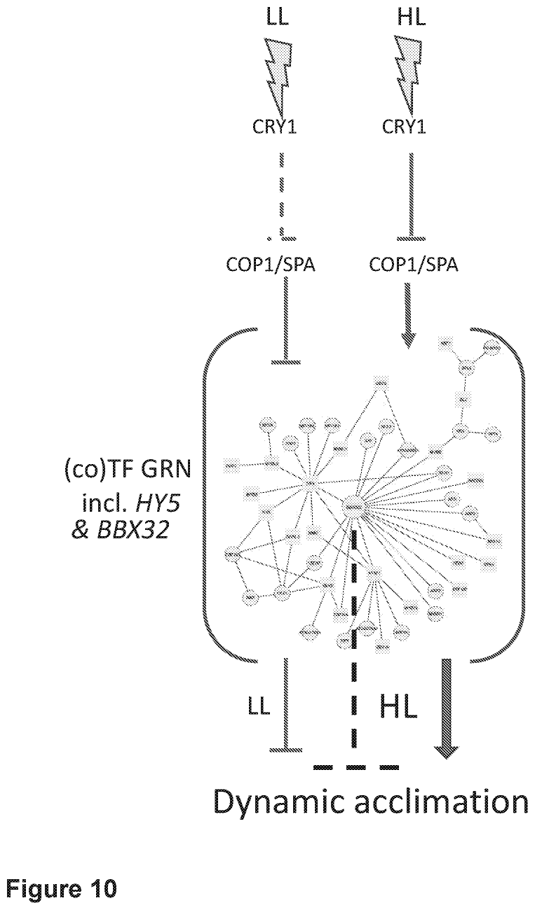

FIG. 10 shows proposed regulation of dynamic acclimation by a BBX32-centric GRN. The above scheme, while was based on this study, incorporates features from schemes published on the photoreceptor-directed control of seedling photomorphogenesis (e.g. Holtan et al., 2011; Hoecker, 2017). Under LL growth conditions to which the plant is acclimated, all or a proportion of cellular CRY1 is not active and consequently COP1/SPA acts to negatively regulate GRN members including HY5 and BBX32. Upon exposure to HL, CRY1 is activated and blocks COP1/SPA, which in turn releases the GRN ultimately leading to the establishment of dynamic acclimation. However, a degree of negative regulation of dynamic acclimation is retained (dotted inverted T) under HL conditions to allow for flexibility in potential fluctuations in the light environment.

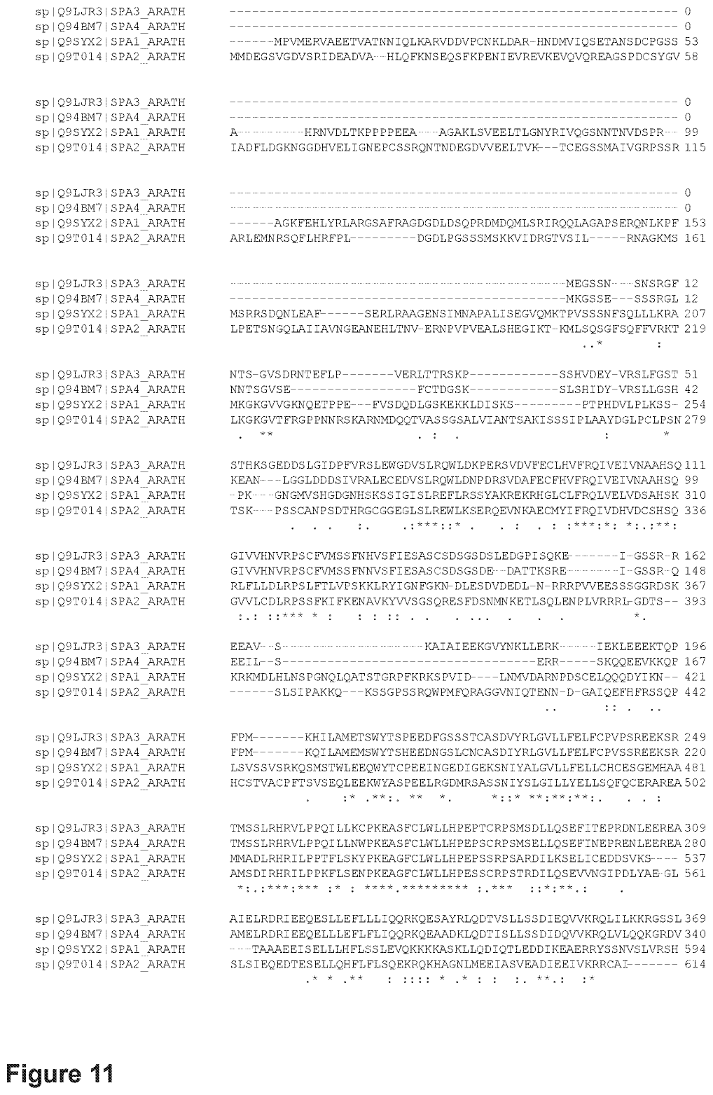

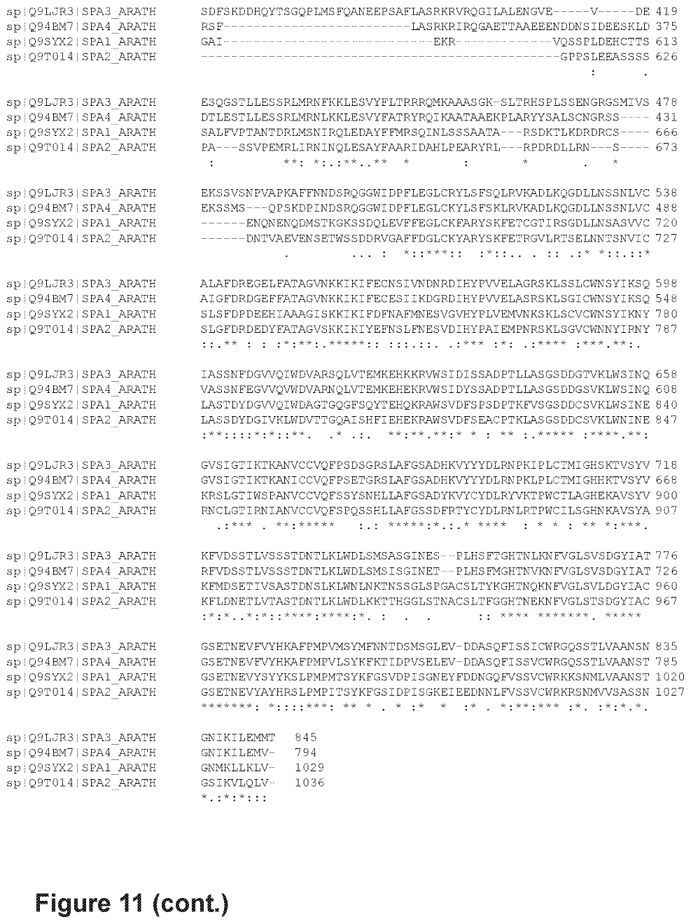

FIG. 11 shows a sequence alignment of SPA1, SPA2, SPA3 and SPA4 proteins in A. thaliana.

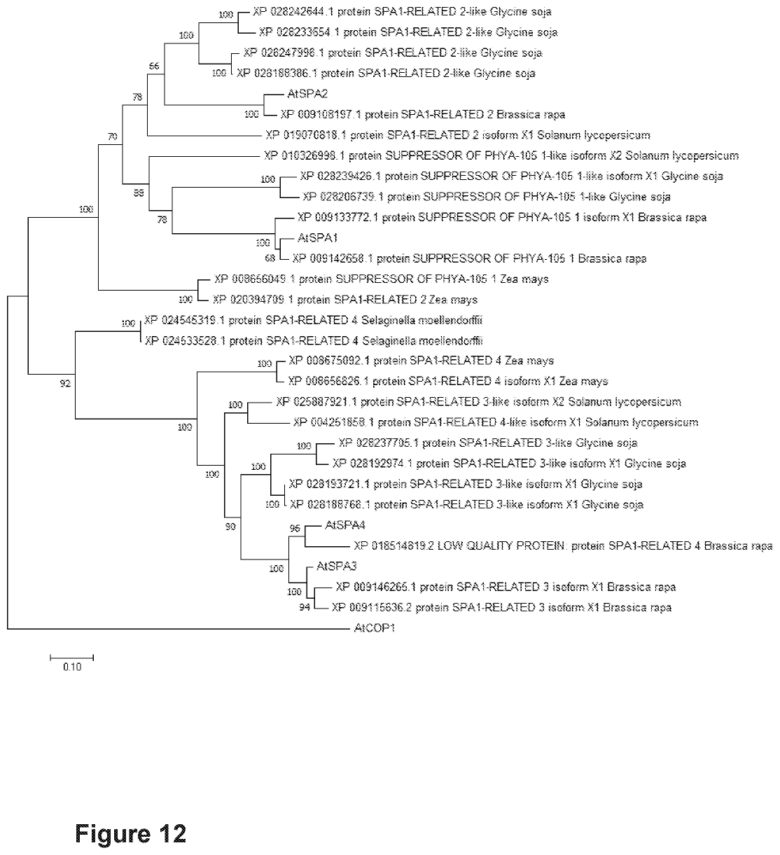

FIG. 12 is a phylogenetic tree diagram, showing that SPA genes are highly conserved across the plant kingdom.

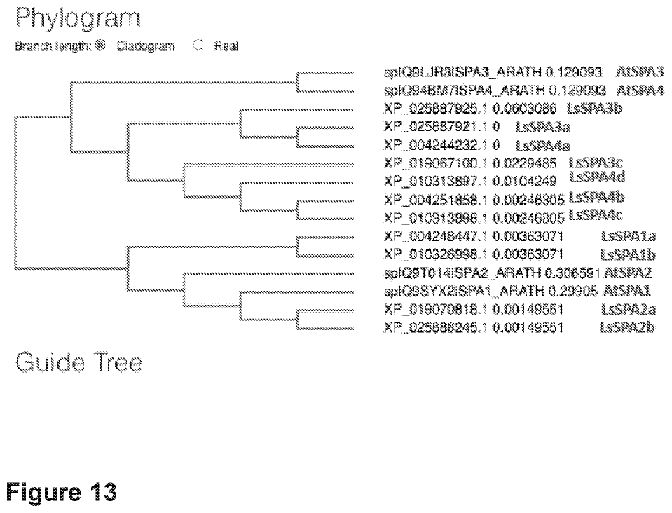

FIG. 13 is a phylogenetic tree diagram, showing SPA protein alignments between A. thaliana and S. lycopersicum (tomato).

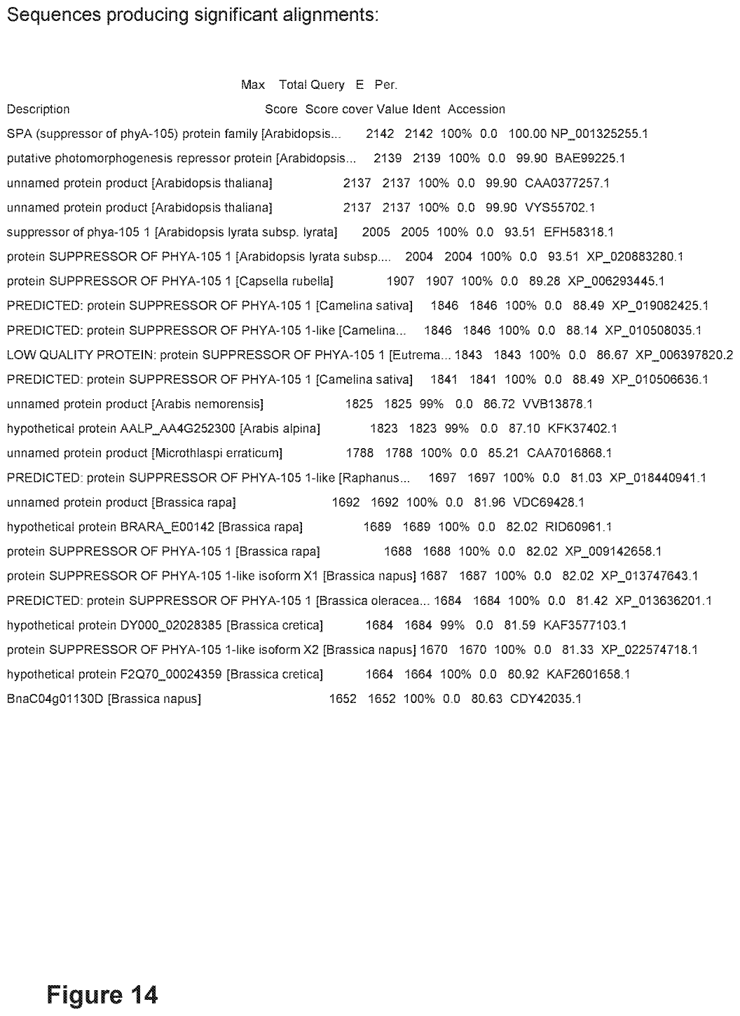

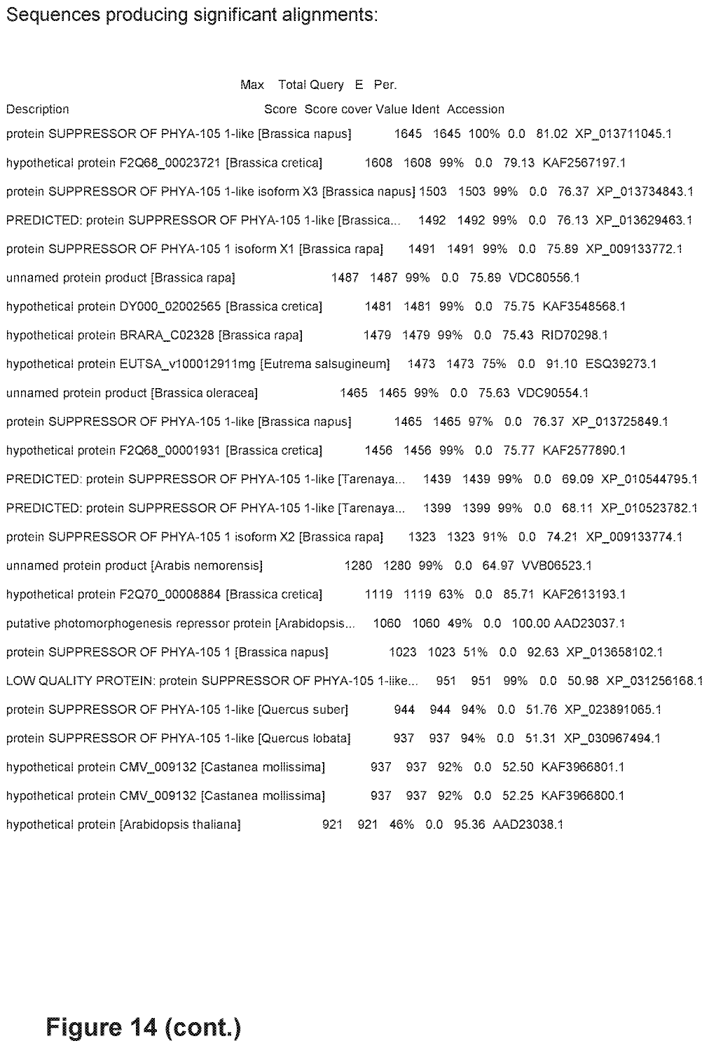

FIG. 14 shows Identification of conserved SPA proteins across different species.

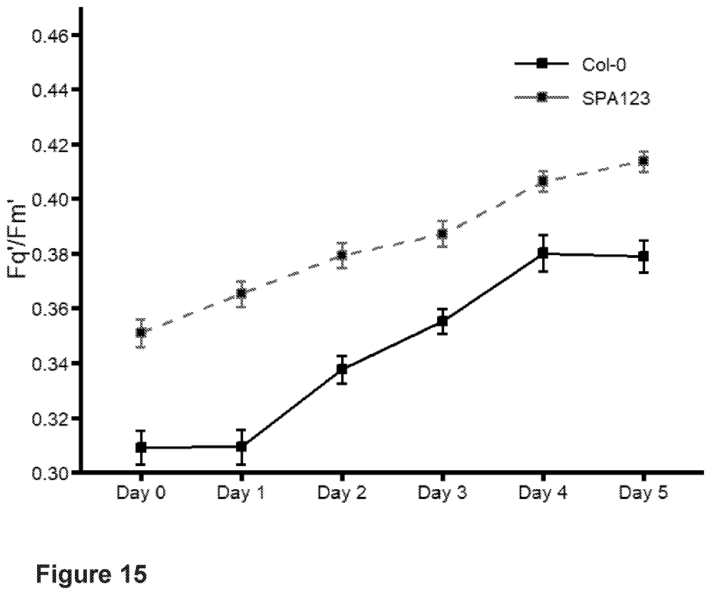

FIG. 15 shows enhanced photosynthetic efficiency of spa1,2,3 triple mutant compared with wild type plants (Col-0). Daily 4 HL exposures of Arabidopsis spa1/spa2/spa3 and Col-0 (wild-type) followed by determination of their photosystem II operating efficiency (Fq′/Fm′), at an actinic PPFD (light intensity) of 800 μmol m⁻² s⁻¹). The differences between wild-type and mutant at every time point were significant (P<0.005; ANOVA and post-hoc Games-Howell).

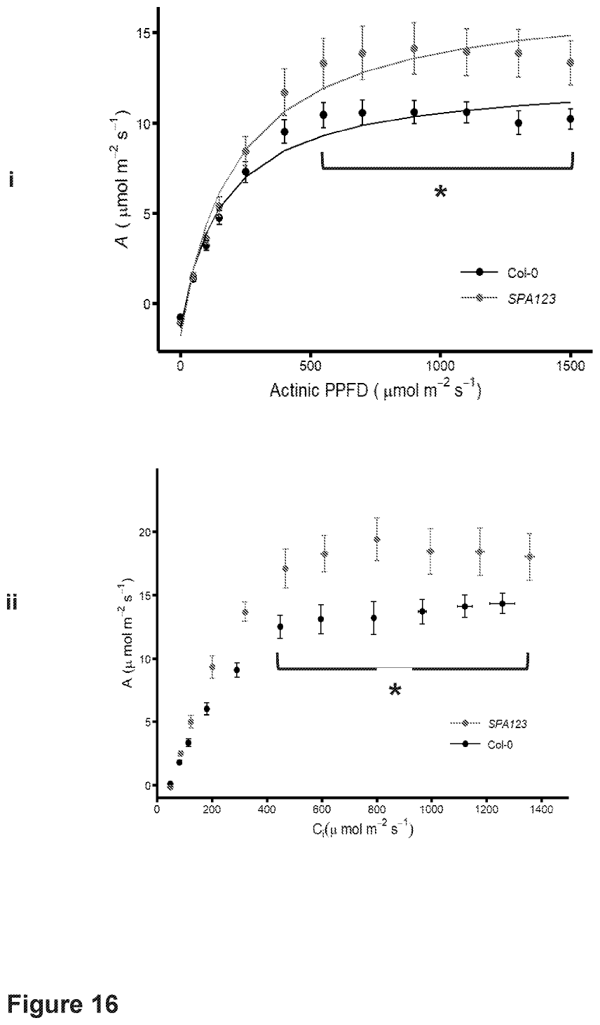

FIG. 16 shows photosynthetic capacity of Arabidopsis spa1,2,3 compared with Col-0 (wild type) as a function of (i) actinic PPFD and (ii) internal leaf CO₂ concentration (Ci). The rate of CO₂ assimilation rate (A) was determined as a function of actinic PPFD and internal leaf CO₂ concentration (Ci) in mature leaves of the mutant wild type plants (mean±SE n=5) PPFDs after 5 days of daily 4 h HL exposure. Asterisks indicates a significant difference between mutant and wild type in photosynthetic capacity ((P<0.02; covariant T and two-tailed F tests). A was determined using Infra-Red Gas Analysis.

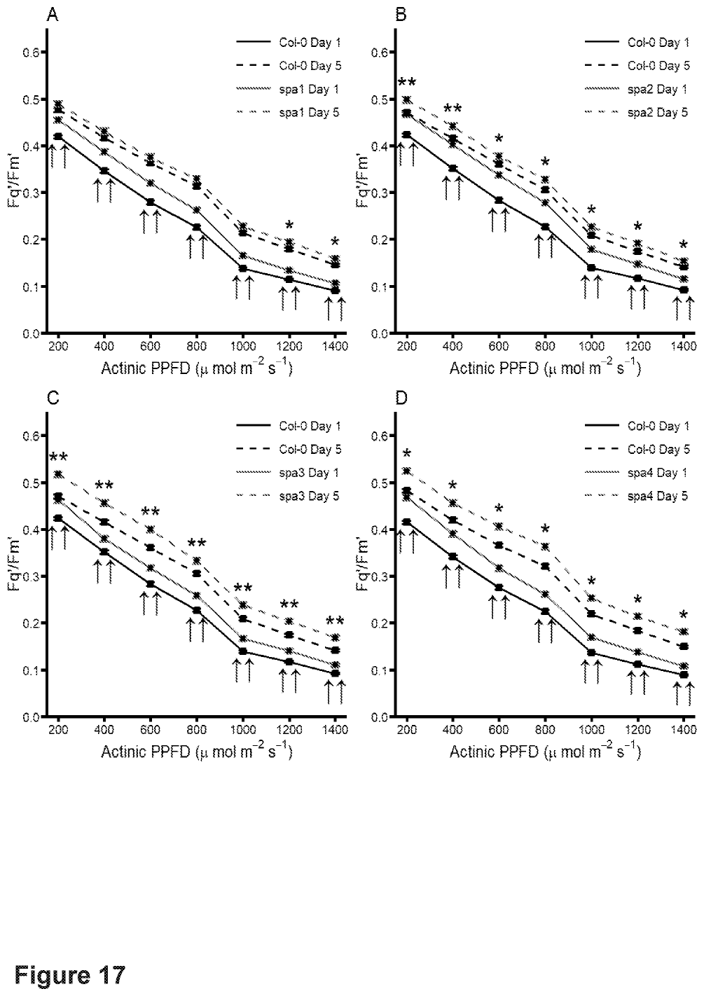

FIG. 17 shows photosynthetic efficiency of spa1 (A), spa2 (B), spa3 (C), spa4 (D) and Col-plants. Fq′/Fm′ was determined by chlorophyll fluorescence with an actinic PPFD range of 200 μmol m⁻² s⁻¹ to 1400 μmol m⁻² s⁻¹ at 200 μmol m⁻² s⁻¹ increments. Measurements were taken from 4-6 week old plants with a minimum of 6 mature leaves each (means±SE). Arrows and asterisks denote a significant difference between the day 1 and day 5 values respectively of the mutants and Col-0 (Anova, post-hoc GH; */↑=p<0.05; **/↑↑=p<0.005).

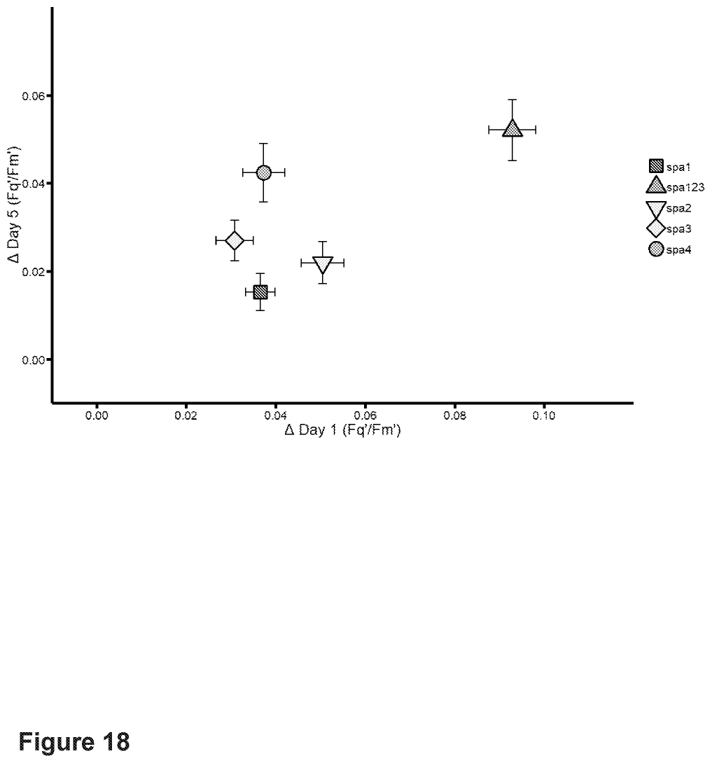

FIG. 18 shows difference in photosynthetic efficiency between Col-0 and spa1, spa2, spa3, spa4, and spa123 plants. ΔFq′/Fm′ was determined by chlorophyll fluorescence with a light intensity of 800 μmol m⁻² s⁻¹ minus the Col-0 (WT) value, as in FIG. 16. Measurements were taken from 4-6 week old plants with a minimum of 6 mature leaves each (means±SE).

### SEQUENCE LISTING

SEQ ID NO: 1 shows full length genomic nucleic acid sequence of SPA1 in A. thaliana.
SEQ ID NO: 2 shows the amino acid sequence of SPA1 protein in A. thaliana.
SEQ ID NO:3 shows full length genomic nucleic acid sequence of SPA2 in A. thaliana.
SEQ ID NO: 4 shows the amino acid sequence of SPA2 protein in A. thaliana.
SEQ ID NO: 5 shows full length genomic nucleic acid sequence of SPA3 in A. thaliana.
SEQ ID NO: 6 shows the amino acid sequence of SPA3 protein in A. thaliana.
SEQ ID NO: 7 shows full length genomic nucleic acid sequence of SPA4 in A. thaliana.
SEQ ID NO: 8 shows the amino acid sequence of SPA4 protein in A. thaliana.
SEQ ID NO: 9 shows full length genomic nucleic acid sequence of BBX32 in A. thaliana.
SEQ ID NO: 10 shows the amino acid sequence of BBX32 protein in A. thaliana.
SEQ ID NO: 11 shows full length genomic nucleic acid sequence of COP1 in A. thaliana.
SEQ ID NO: 12 shows the amino acid sequence of COP1 protein in A. thaliana.
SEQ ID NO: 13 shows full length genomic nucleic acid sequence of CRY1 in A. thaliana.
SEQ ID NO: 14 shows the amino acid sequence of CRY1 protein in A. thaliana.
SEQ ID NO: 15 shows full length genomic nucleic acid sequence of HY5 in A. thaliana.
SEQ ID NO: 16 shows the amino acid sequence of HY5 protein in A. thaliana.
SEQ ID NO: 17 shows full length genomic nucleic acid sequence of PIF4 in A. thaliana.
SEQ ID NO: 18 shows the amino acid sequence of PIF4 protein in A. thaliana.
SEQ ID NO: 19 shows full length genomic nucleic acid sequence of PHYB in A. thaliana.
SEQ ID NO: 20 shows the amino acid sequence of PHYB protein in A. thaliana.
SEQ ID NO: 21 shows a first amino acid sequence of a SPA1 homolog in S. lycopersicum.
SEQ ID NO: 22 shows a second amino acid sequence of a SPA1 homolog in S. lycopersicum.

The practice of embodiments of the present invention employs, unless otherwise indicated, conventional techniques of chemistry, molecular biology, which are within the skill of those working in the art.

Most general chemistry techniques can be found in Comprehensive Heterocyclic Chemistry (Katritzky et al., 1996, published by Pergamon Press); Comprehensive Organic Functional Group Transformations (Katritzky et al., 1995, published by Pergamon Press); Comprehensive Organic Synthesis (Trost et al., 1991, published by Pergamon); Heterocyclic Chemistry (Joule et al. published by Chapman & Hall); Protective Groups in Organic Synthesis (Greene et al., 1999, published by Wiley-Interscience); and Protecting Groups (Kocienski et al., 1994).

Most general molecular biology techniques can be found in Sambrook et al, Molecular Cloning, A Laboratory Manual (2001) Cold Harbor-Laboratory Press, Cold Spring Harbor, N.Y. or Ausubel et al., Current Protocols in Molecular Biology (1990) published by John Wiley and Sons, N.Y.

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2^{nd} ed., 2002, CRC Press; The Dictionary of Cell and Molecular Biology, 3^{rd} ed., Academic Press; and the Oxford University Press, provide a person skilled in the art with a general dictionary of many of the terms used in this disclosure. For chemical terms, the skilled person may refer to the International Union of Pure and Applied Chemistry (IUPAC).

Units, prefixes and symbols are denoted in their Système International d'Unités (SI) accepted form. Numeric ranges are inclusive of the numbers defining the range.

### Methods of Increasing Photosynthetic Capacity in a Plant

In certain embodiments, the invention provides a method of increasing photosynthetic capacity in a plant (e.g., the mature leaves of a plant). As used herein, increased photosynthetic capacity refers to increased electron transport and/or carbon metabolism. Typically, increased photosynthetic capacity does not result in the abundance of photosynthetic reaction centres.

As used herein, “photosynthetic capacity” may also refer to the biochemical capacity for photosynthesis in the plant.

In certain embodiments, leaves of plants transferred from low light (LL) to high light (HL) conditions have an increased capacity for photosynthesis (e.g. “dynamic acclimation” as further described herein).

As used herein, “Low light (LL)” may refer to dark-adapted conditions. For example, LL conditions may relate to a light intensity (PPFD) of about 100 μmol m⁻² s⁻² or less, about 50 μmol m⁻² s⁻² or less, or about 10 μmol m⁻² s⁻² or less. Typically, the LL conditions are about 100 μmol m⁻² s⁻² (e.g. between 90 to 110 μmol m⁻² s⁻²).

As used herein, “high light (HL)” may refer to increased light intensity as compared to LL. For example, HL conditions may relate to a light intensity (PPFD) of about 200 μmol m⁻² s⁻² or more, about 400 μmol m⁻² s⁻² or more, about 600 μmol m⁻² s⁻² or more, about 800 μmol m⁻² s⁻² or more, about 1000 μmol m⁻² s⁻² or more, about 1200 μmol m⁻² s⁻² or more or about 1400 μmol m⁻² s⁻² or more. Typically, the HL conditions are about 1100 μmol m⁻² s⁻² (e.g. between 1000 to 1200 μmol m⁻² s⁻²).

In certain embodiments, “increasing photosynthetic capacity” may relate to increasing photosystem II (PSII) operating efficiency, linear electron flux and/or quantum yield of CO₂ assimilation.

In certain embodiments, “increasing photosynthetic capacity” in a plant may relate to increasing maximum quantum efficiency of PSII photochemistry (Fᵥ/Fₘ). As described herein, this relates to the maximum efficiency at which light absorbed by PSII is used for reduction of the primary quinone acceptor of PSII (Q_{A}).

In certain embodiments, “increasing photosynthetic capacity” in a plant may relate to increasing PSII operating efficiency (F_{q}′/Fₘ′). As described herein, this relates to the efficiency at which light absorbed by PSII is used for Q_{A} reduction. At a given photosynthetically active photon flux density (PPFD) this parameter provides an estimate of the quantum yield of linear electron flux through PSII.

In certain embodiments, “increasing photosynthetic capacity” in a plant may relate to increasing the PSII efficiency factor (F_{q}′/Fᵥ′). As described herein, this relates the PSII maximum efficiency to the PSII operating efficiency. It is nonlinearly related to the proportion of PSII centers that are “open” (Q_{A} oxidized).

Photosynthetic capacity may be measured in plants using any suitable technique. For example, chlorophyll fluorescence (CF) may be used to determine photosynthetic capacity as well described in the art (see, for example, Baker et al, 2008, Annu. Rev. Plant Biol. 2008.59:89-113). CF imaging systems to determine photosynthetic capacity are commercially available (e.g. Flourimager, Technologica Ltd. or the like).

In certain embodiments, the invention provides a method of increasing photosynthetic capacity in a plant, the method comprising modulating CRY1-directed signalling in the plant, wherein photosynthetic capacity in the plant is determined to be increased as compared to a control plant or reference value.

For example, any method as described herein may comprise the step of determining photosynthetic capacity of the plant (e.g., in mature leaves of the plant). Typically, the method may comprise quantifying PSII operating efficiency, linear electron flux and/or quantum yield of CO₂ assimilation in mature leaves of the plant. Typically, the photosynthetic capacity of the plant (e.g., PSII operating efficiency, linear electron flux and/or quantum yield of CO₂ assimilation) is compared to the (equivalent) photosynthetic capacity of one or more control plants or reference values as further described herein.

As used herein, “mature leaves” refer to fully expanded leaves of the plant (e.g., so-called photosynthate source leaves that drive plant productivity).

In certain embodiments, plants are subject to HL (e.g. for about 4 hours), followed by LL (e.g. for about 0.5 hours), and subsequently exposed to a range of actinic PPFDs (e.g. for about minutes) to collect chlorophyll fluorescence (CF) measurements.

As used herein, “actinic light” refers to light that is absorbed by the photosynthetic apparatus and will drive electron transport. Typically, plants are exposed to increasing actinic PPFD from about 200-to about 1400 μmol m⁻² s⁻² in about 200 μmol m⁻² s⁻² steps about every 5 min (see, for example, Barbagallo et al., 2003; Gorecka et al., 2014).

In certain embodiments, the plant has increased photosynthetic capacity relative to a control or wild-type plant. For example, the plant may have increased photosynthetic capacity as compared to plants with non-modulated CRY1-directed signaling in the plant. The plant may have increased photosynthetic capacity as compared to a reference value obtained from a control or wild-type plant.

In certain embodiments, the plant has an increased photosynthetic capacity of about 10%, 15%, 20%, 25%, 30%, 35%, 40%, 45%, 50%, 55%, 60%, 70%, 80%, 90%, 100%, 200%, 500% or more as compared to a control or wild-type plant. Typically, the plant has an increased photosynthetic capacity of about 40% or more as compared to the control or wild-type plant.

In certain embodiments, the PSII operating efficiency (Fq′/Fm′) is increased by about 40% or more as compared to a control or wild-type plant. Typically, the PSII operating efficiency is determined at a PFFD of about 800 μmol m⁻² s⁻¹.

A control or wild-type plant may be a plant which has not been modified according to the methods of the invention. For example, the control or wild type plant may have normal (e.g. unaltered) CRY1-directed signaling. In particular, the control plant may have normal or unaltered levels of BBX32, SPA (e.g. SPA1, 2, 3 and/or 4), COP, CRY1, HY5, PIF (e.g. PIF1, 3, 4 and/or 5) and/or PHYB grown under similar or the same conditions. Typically, the control or wild-type plant is of the same plant species, preferably having the same genetic background as the modified plant.

As used herein, the term “plant” refers to all genera and species of higher and lower plants of the Plant Kingdom. The term includes the mature plants, seeds, shoots, seedlings, and part, propagation material, plant organ tissue, protoplasts, callus and other cultures, for example cell cultures, derived from them, and all other species of groups of plant cells giving functional or structural units. Mature plants refer to plants at any developmental stage beyond the seedling. Seedling refers to a young, immature plant at an early developmental stage.

In certain embodiments, the plant is a monocot or a dicot.

In certain embodiments, the plant is a model species such as A. thaliana.

In certain embodiments, the plant is a crop plant. A crop plant is any plant which is grown on a commercial scale for human or animal consumption or use.

In certain embodiments, the crop plant is a C₃ plant. Alternatively, the crop plant may be a C₄ plant.

In certain embodiments, the plant is a cereal crop such as Triticum species (e.g. wheat), Oryza sativa (e.g. rice), Glycine max (e.g. soybean), barley, rye, oats, sorghum, alfalfa, clover, Zea mays (maize) and the like. The plant may be an oil-producing plant such as canola, safflower, sunflower, peanut, cacao and the like. The plant may be tobacco. The plant may be a vegetable crop such as tomato, potato, pepper, eggplant, sugar beet, carrot, cucumber, lettuce or pea. The plant may be a Brassica species such as B. campestris, B. napus, B. rapa or B. carinata. The plant may be Cannabis sativa (e.g. hemp), Carthamus tinctorius (e.g. safflower), Linum usitatissimum (e.g. linseed or flax) or Olea europaea (olive).

In certain embodiments, the plant is wheat. For example, the wheat may be a hexaploid species such as T. aestivum or T. spelta. The wheat may be a tetraploid species such as T. durum or T. dicoccon. Typically, the wheat is hexaploid.

In certain embodiments, the plant is grown under field conditions. Advantageously, plants having increased photosynthetic capacity as described herein may have increased fitness to a changing environment as compared to compared to non-modified plants.

In certain embodiments, the plant is grown under controlled light conditions (e.g. in a greenhouse). For example, the plant may be a tomato. Typically, the plant is grown under controlled light conditions. For example, light conditions (e.g. light intensity and/or duration) may be optimised depending on the photosynthetic capacity of the plant. Advantageously, plants having increased photosynthetic capacity as described herein may be grown under controlled conditions using less energy as compared to non-modified plants whilst retaining equivalent or even improved yield.

### CRY1-Directed Signalling Pathways

In certain embodiments, the method of increasing photosynthetic capacity in the plant comprises modulating CRY1-directed signalling in the plant.

As used herein, “modulating CRY1-directed signalling” specifically refers to targeting the expression and/or activity of one or more genes that are involved in the transcription regulation of dynamic acclimation upon exposure to HL.

In certain embodiments, the method comprises removing a constraint on maximum photosynthetic capacity in the plant. For example, the method may comprise reducing or abolishing the expression or activity of any negative regulator of photosynthetic capacity as described herein.

As used herein, the term “reducing” means a decrease in the levels of expression and/or activity (e.g. biological activity) of a gene (e.g. SPA, BBX32 and/or COP) or corresponding protein (e.g. SPA, BBX32 and/or COP) by about 10%, 20%, 30%, 40%, 50%, 60%, 70%, 80%, 90% or more. The term “abolishing” means that substantially no or no expression of a gene (e.g. SPA, BBX32 and/or COP) is detectable and/or that substantially no or no functional polypeptide (e.g. SPA, BBX32 and/or COP) is produced. Any method for determining the level and/or activity of a gene or corresponding protein may be employed, as well known to the skilled person.

### SPA Genes

In preferred embodiments, the method comprises reducing or abolishing the expression of at least one nucleic acid sequence encoding a Suppressor of PhyA-105 (SPA) polypeptide and/or reducing or abolishing the activity of a SPA polypeptide in the plant.

The method may comprise reducing or abolishing the expression of any number of SPA genes. By way of example only, there are four SPA genes in Arabidopsis. The method of the invention may involve reducing the expression of one, two, three or four nucleic acid sequences encoding SPA1, SPA2, SPA3 and/or SPA4.

In certain embodiments, the method comprises reducing or abolishing the expression of one, two or three nucleic acid sequences encoding SPA polypeptides. For example, the invention relates to the following mutations:

| Single mutants | Double mutants | Triple mutants |
|---|---|---|
| spa 1 | spa 1/spa 2 | spa 1/spa 2/spa 3 |
| | spa 1/spa 3 | spa 1/spa 2/spa 4 |
| | spa 1/spa 4 | spa 1/spa 3/spa 4 |
| spa 2 | spa 2/spa 3 | spa 2/spa 3/spa 4 |
| | spa 2/spa 4 | |
| spa 3 | spa 3/spa 4 | — |
| spa 4 | — | — |

As used herein, reference to reduced or abolished SPA expression and/or activity may refer to any single, double or triple mutant shown above.

In certain embodiments, methods may comprise determining the minimum number of SPA genes that may need to be inactivated to remove constraints on maximum photosynthetic capacity in any given species (e.g. crop species), whilst avoiding or minimising any significant impact on plant stature or morphology. Advantageously, the invention therefore allows determination of the best combination of SPA genes to remove to achieve maximum photosynthetic capacity whilst minimising other possible negative effects on the plant.

In certain embodiments, shoot growth of the plant remains significantly the same (or is improved) as compared to a control or wild type plant. In other words, by targeting the minimum number of SPA genes to achieve increased photosynthetic capacity any dwarfed phenotype may be avoided.

In certain embodiments, the method may comprise reducing or abolishing the expression of all four SPA genes (e.g. spa1/spa2/spa3/spa4). Typically, such plants are dwarf as compared to the single, double or triple mutants as described herein.

The nucleic acid sequence may be any sequence associated with expression of SPA (e.g. SPA1, SPA2, SPA 3 and/or SPA4 or any homolog as further described herein).

In certain embodiments, the nucleic acid sequence is a coding sequence. For example, the nucleic acid sequence may be genomic DNA (e.g. including exons and/or introns).

In certain embodiments, the nucleic acid sequence is mRNA or cDNA.

In certain embodiments, the nucleic acid sequence is non-coding regulatory sequence. For example, the non-coding regulatory sequence may be a promoter of SPA. A promoter of SPA may comprise nucleic acid sequence extending for at least about 2 kbp, 3 kbp, 4 kbp, 5 kbp or more upstream of the ATG codon of the SPA open reading frame. The non-coding regulatory sequence may be an enhancer of SPA expression. The promoter may be a SPA1, SPA2, SPA3 and/or SPA4 promoter.

In certain embodiments, the nucleic acid sequence comprises SEQ ID NO: 1, 3, 5, 7 or a homolog or variant thereof as described herein. In certain embodiments, the nucleic acid sequence encodes the amino acid sequences set forth in SEQ ID NO: 2, 4, 6, 8 or a homolog or variant thereof as described herein.

As used herein, the term “homolog” refers to a polypeptide or polynucleotide sequence possessing a high degree of sequence relatedness to a subject sequence. Such relatedness may be quantified by determining the degree of identity and/or similarity between the sequences being compared.

As used herein, the term “homolog” includes any “homeolog”. This refers to multiple copies of a gene in a plant's genome derived from different species as a result of allopolyploidy. Where multiple genes are identified corresponding to only one gene in a diploid species such as Arabidopsis or tomato, the skilled person would understand how to reduce or abolish expression and/or activity of all homeologs using techniques known in the art and described herein.

The one or more SPA genes may be derived from any plant species including any of the species described elsewhere herein.

In certain embodiments, the nucleic acid sequence is at least about 70%, 80%, 90%, 95%, 96%, 97%, 98%, 99% or 100% identical to any one of SEQ ID NO: 1, 3, 5 or 7.

In certain embodiments, the nucleic acid sequence encodes an amino acid at least about 70%, 80%, 90%, 95%, 96%, 97%, 98%, 99% or 100% identical to SEQ ID NO: 2, 4, 6 or 8.

In certain embodiments, the invention provides a method of increasing photosynthetic capacity by reducing or abolishing the activity of a SPA polypeptide in the plant.

In certain embodiments, the SPA polypeptide comprises the amino acid sequences set forth in SEQ ID NO: 2, 4, 6 or 8 or a homolog or variant thereof as described herein. In certain embodiments, the amino acid has at least about 70%, 80%, 90%, 95%, 96%, 97%, 98%, 99% or 100% identity to SEQ ID NO: 2, 4, 6 or 8.

As used herein, the term “percent sequence identity” is the percentage of nucleic acids or amino acids that are identical when the two sequences are compared. Homology or sequence identity of two nucleic or amino acid sequences may be determined by methods known in the art. For example, the sequence identity may be determined using the algorithm of Karlin and Altschul (1990) Proc. Natl. Acad. Sci., U.S.A 87: 2264-2268. Such an algorithm is incorporated into the NBLAST and XBLAST programs of Altschul et al. (1990) J. Mol. Biol. 215: 403-410. BLAST nucleotide searches are performed with the NBLAST program, score=100, word length 12, to obtain nucleotide sequences homologous to a nucleic acid molecules of certain embodiments of the present invention. BLAST protein sequences are performed with the XBLAST program, score=50, word length=3, to obtain amino acid sequences homologous to a reference polypeptide. To obtain gapped alignments for comparison purposes, Gapped BLAST is utilised as described in Altshul et al. (1997) Nucleic Acids Res. 25: 3389-3402. When utilising BLAST and Gapped BLAST programs, the default parameters are typically used (See http://www.ncbi.nlm.nih.gov).

Suitable homologues can be identified by sequence comparison and identification of conserved domains. There are predictors in the art that can be used to identify such sequences. The function of the homologue can be identified using methods described herein and a skilled person would thus be able to confirm the function. For example, the homolog may be overexpressed in a plant to confirm function.

In certain embodiments, the nucleotide sequences described herein (e.g. SEQ ID NO: 1, 3, 5 or 7) may be used to isolate corresponding sequences from other plants, for example other crops. For example, PCR, hybridization or other techniques may be used to identify corresponding sequences based on their sequence homology to the sequences described herein.

In certain embodiments, topology of the sequences and/or characteristic domains may be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

In certain embodiments, homologous sequences may be confirmed by hybridization, wherein the hybridization takes place under stringent conditions. Using the stringent hybridization (i.e. washing the nucleic acid fragments twice where each wash is at room temperature for 30 minutes with 2× sodium chloride and sodium citrate (SCC buffer; 1.150. mM sodium chloride and 15 mM sodium citrate, pH 7.0) and 0.1% sodium dodecyl sulfate (SDS); followed by washing one time at 50° C. for 30 minutes with 2×SCC and 0.1% SDS; and then washing two times where each wash is at room temperature for 10 minutes with 2×SCC), homologous sequences can be identified comprising at most about 25 to about 30% base pair mismatches, or about 15 to about 25% base pair mismatches, or about 5 to about 15% base pair mismatches.

In certain embodiments, the one or more SPA genes are derived from tomato. By way of example, DNA sequence for tomato may be recovered by several approaches, including PCR, screening of libraries or gene synthesis.

In certain embodiments, the nucleic acid sequence may encode an amino acid at least about 70%, 80%, 90%, 95%, 96%, 97%, 98%, 99% or 100% identical to SEQ ID NO: 21 or 22. In certain embodiments, the SPA polypeptide comprises the amino acid sequences set forth in SEQ ID NO: 21 or 22 or a homolog or variant thereof as described herein.

### BBX32 Genes

In certain embodiments, modulating CRY1-directed signalling comprises reducing or abolishing the expression of at least one nucleic acid sequence encoding a B-Box Domain containing protein 32 (BBX32) polypeptide and/or reducing or abolishing the activity of a BBX32 polypeptide in the plant.

In certain embodiments, the nucleic acid sequence comprises SEQ ID NO: 9 or a homolog or variant thereof as described herein. In certain embodiments, the nucleic acid sequence encodes the amino acid sequences set forth in SEQ ID NO: 10 or a homolog or variant thereof as described herein.

The one or more BBX32 genes may be derived from any plant species including any of the species described elsewhere herein.

In certain embodiments, the nucleic acid sequence is at least about 70%, 80%, 90%, 95%, 96%, 97%, 98%, 99% or 100% identical to SEQ ID NO: 9. In certain embodiments, the nucleic acid sequence encodes an amino acid at least about 70%, 80%, 90%, 95%, 96%, 97%, 98%, 99% or 100% identical to SEQ ID NO: 10.

In certain embodiments, the invention provides a method of increasing photosynthetic capacity by reducing or abolishing the activity of a BBX32 polypeptide in the plant.

In certain embodiments, the BBX32 polypeptide comprises the amino acid sequences set forth in SEQ ID NO: 10 or a homolog or variant thereof as described herein. In certain embodiments, the amino acid has at least about 70%, 80%, 90%, 95%, 96%, 97%, 98%, 99% or 100% identity to SEQ ID NO: 10.

### COP Genes

In certain embodiments, modulating CRY1-directed signalling comprises reducing or abolishing the expression of at least one nucleic acid sequence encoding a Constitutively Photomorphogenic (COP) polypeptide (e.g. COP1) and/or reducing or abolishing the activity of a COP polypeptide (e.g. COP1) in the plant.

In certain embodiments, COP expression and/or activity may be reduced rather than abolished. For example, shoot growth of the plant may remain significantly the same (or be improved) as compared to a control or wild type plant. In other words, by reducing (rather than abolishing) COP expression, any dwarfed phenotype may be avoided.

In certain embodiments, the nucleic acid sequence comprises SEQ ID NO: 11 or a homolog or variant thereof as described herein. In certain embodiments, the nucleic acid sequence encodes the amino acid sequences set forth in SEQ ID NO: 12 or a homolog or variant thereof as described herein.

The one or more COP genes may be derived from any plant species including any of the species described elsewhere herein.

In certain embodiments, the nucleic acid sequence is at least about 70%, 80%, 90%, 95%, 96%, 97%, 98%, 99% or 100% identical to SEQ ID NO: 11. In certain embodiments, the nucleic acid sequence encodes an amino acid at least about 70%, 80%, 90%, 95%, 96%, 97%, 98%, 99% or 100% identical to SEQ ID NO: 12.

In certain embodiments, the invention provides a method of increasing photosynthetic capacity by reducing or abolishing the activity of a COP polypeptide in the plant.

In certain embodiments, the COP polypeptide comprises the amino acid sequences set forth in SEQ ID NO: 12 or a homolog or variant thereof as described herein. In certain embodiments, the amino acid has at least about 70%, 80%, 90%, 95%, 96%, 97%, 98%, 99% or 100% identity to SEQ ID NO: 12.

### Positive Regulators of CRY-1 Directed Signalling

In certain embodiments, the method comprises introducing and/or increasing the expression of a positive regulator of photosynthetic capacity. Positive regulators of CRY-1 directed signalling disclosed herein, include, for example, CRY1, HY5, PIF and/or PHYB.

In certain embodiments, a positive regulator of CRY-1 directed signalling may be isolated from a plant and inserted into a vector/expression cassette for transformation, e.g. by using an artificial plant chromosome. The positive regulator may be isolated from any suitable plant species, and inserted into any suitable vector and/or expression system as well described in the art.

In certain embodiments, a derivative of a positive regulator of CRY-1 directed signalling is used. For example, a positive regulator of CRY-1 directed signalling (e.g. CRY1, HY5, PIF and/or PHYB) may be modified using any suitable technique so that the resulting protein is constitutively active (e.g. cannot be inhibited by negative regulators of the CRY-1 directed signalling pathway as described herein).

In certain embodiments, a positive regulator of CRY-1 directed signalling is a transgene that is introduced in the plant. This can be carried out by any suitable technique described in the art, e.g. using transformation with Agrobacterium, particle bombardment, or the like.

In certain embodiments, the positive regulator of CRY-1 directed signalling is an exogenous gene, such as one or more Arabidopsis gene or variant thereof as described herein, overexpressed in a different plant species. Alternatively, the positive regulator of CRY-1 directed signalling is an endogenous gene, i.e. a gene that is endogenous to the plant in which it is introduced and overexpressed.

As described herein, “overexpression” refers to expression of a positive regulator of CRY-1 directed signalling (e.g. CRY1, HY5, PIF and/or PHYB) at a level that is higher than expression driven by its endogenous promoter. For example, overexpression may be carried out using a strong promoter, such as the cauliflower mosaic virus promoter (CaMV35S), the rice actin promoter or the maize ubiquitin promoter or any promoter that gives enhanced expression. Alternatively, enhanced or increased expression can be achieved by using transcription or translation enhancers or activators and may incorporate enhancers into the gene to further increase expression. Furthermore, an inducible expression system may be used, such as a steroid or ethanol inducible expression system. The coding sequence may be on a monocistronic or polycistronic messenger RNA. Also envisaged is ectopic expression, i.e. gene expression in a tissue in which it is normally not expressed.

### CRY1 Genes

In certain embodiments, the positive regulator of CRY-1 directed signalling comprises an amino acid sequence as set forth in SEQ ID NO: 14.

The one or more CRY1 genes may be derived from any plant species including any of the species described elsewhere herein.

In certain embodiments, the amino acid has at least about 70%, 80%, 90%, 95%, 96%, 97%, 98%, 99% or 100% identity to SEQ ID NO: 14.

In certain embodiments, the method comprises introducing into the plant a nucleic acid molecule comprising a nucleic acid sequence selected from:
a) a nucleic acid sequence as set forth in SEQ ID NO: 13;
b) a nucleic acid sequence having at least about 70%, 80%, 90%, 95%, 96%, 97%, 98%, 99% or 100% identity to SEQ ID NO: 14; or
c) a nucleic acid sequence which hybridises to (a) or (b) and which encodes a CRY1 protein; or
d) a nucleic acid sequence which differs from (a), (b) or (c) by virtue of the degeneracy of the genetic code and which encodes a CRY1 protein.

### HY5 Genes

In certain embodiments, the positive regulator of CRY-1 directed signalling comprises an amino acid sequence as set forth in SEQ ID NO: 16.

The one or more HY5 genes may be derived from any plant species including any of the species described elsewhere herein.

In certain embodiments, the amino acid has at least about 70%, 80%, 90%, 95%, 96%, 97%, 98%, 99% or 100% identity to SEQ ID NO: 16.

In certain embodiments, the method comprises introducing into the plant a nucleic acid molecule comprising a nucleic acid sequence selected from:
a) a nucleic acid sequence as set forth in SEQ ID NO: 15;
b) a nucleic acid sequence having at least about 70%, 80%, 90%, 95%, 96%, 97%, 98%, 99% or 100% identity to SEQ ID NO: 16; or
c) a nucleic acid sequence which hybridises to (a) or (b) and which encodes a HY5 protein; or
d) a nucleic acid sequence which differs from (a), (b) or (c) by virtue of the degeneracy of the genetic code and which encodes a HY5 protein.

### PIF Genes

In certain embodiments, the positive regulator of CRY-1 directed signalling comprises an amino acid sequence as set forth in SEQ ID NO: 18.

The one or more PIF genes may be derived from any plant species including any of the species described elsewhere herein.

In certain embodiments, the amino acid has at least about 70%, 80%, 90%, 95%, 96%, 97%, 98%, 99% or 100% identity to SEQ ID NO: 18.

In certain embodiments, the method comprises introducing into the plant a nucleic acid molecule comprising a nucleic acid sequence selected from:
a) a nucleic acid sequence as set forth in SEQ ID NO: 17;
b) a nucleic acid sequence having at least about 70%, 80%, 90%, 95%, 96%, 97%, 98%, 99% or 100% identity to SEQ ID NO: 18; or
c) a nucleic acid sequence which hybridises to (a) or (b) and which encodes a PIF protein; or
d) a nucleic acid sequence which differs from (a), (b) or (c) by virtue of the degeneracy of the genetic code and which encodes a PIF protein.

### PHYB Genes

In certain embodiments, the positive regulator of CRY-1 directed signalling comprises an amino acid sequence as set forth in SEQ ID NO: 20.

The one or more PHYB genes may be derived from any plant species including any of the species described elsewhere herein.

In certain embodiments, the amino acid has at least about 70%, 80%, 90%, 95%, 96%, 97%, 98%, 99% or 100% identity to SEQ ID NO: 20.

In certain embodiments, the method comprises introducing into the plant a nucleic acid molecule comprising a nucleic acid sequence selected from:
a) a nucleic acid sequence as set forth in SEQ ID NO: 19;
b) a nucleic acid sequence having at least about 70%, 80%, 90%, 95%, 96%, 97%, 98%, 99% or 100% identity to SEQ ID NO: 19; or
c) a nucleic acid sequence which hybridises to (a) or (b) and which encodes a PHYB protein; or
d) a nucleic acid sequence which differs from (a), (b) or (c) by virtue of the degeneracy of the genetic code and which encodes a PHYB protein.

### Modulating Gene Expression

The present invention relates in part to the elucidation of a novel gene regulatory network involved in dynamic acclimation of plants to high light and the inventor's insight that modulating CRY1-directed pathways (e.g. especially through deletion of one or more SPA genes) enhances photosynthetic capacity without adversely effecting other aspects of plant development.

In particular, data described herein using Arabidopsis mutants shows that removing constraints of maximal photosynthesis capacity (e.g. especially through deletion of one or more SPA genes) increases photosynthetic capacity. Based on this surprising insight, the skilled person would be able to put the invention into effect into a range of crop species following the methods described herein.

In certain embodiments, the methods described herein comprise introducing at least one mutation (e.g. loss of function mutation) into at least one nucleic acid sequence encoding SPA (e.g., SPA1, SPA2, SPA3 and/or SPA4), BBX32 and/or COP (e.g. COP1).

In certain embodiments, the methods described herein comprise introducing at least one mutation (e.g. gain of function) into at least one nucleic acid sequence encoding CRY1, HY5, PIF and/or PHYB.

As discussed herein, the nucleic acid sequence may comprise genomic DNA, mRNA, cDNA or non-coding regulatory sequence such as a promoter of the relevant one or more gene(s).

In certain embodiments, mutation(s) are introduced into one or more SPA nucleic acid sequence(s) of SEQ ID NO: 1, 3, 5, 7 or a homolog and/or variant thereof as described herein.

In certain embodiments, mutation(s) are introduced into a nucleic acid sequence encoding one or more SPA amino acid sequence of SEQ ID NO: 2, 4, 6, 8 or a homolog and/or variant thereof as described herein.

In certain embodiments, mutation(s) are introduced into one or more BBX32 nucleic acid sequence(s) of SEQ ID NO: 9 or a homolog and/or variant thereof as described herein. In certain embodiments, mutation(s) are introduced into a nucleic acid sequence encoding one or more BBX32 amino acid sequences of SEQ ID NO: 10 or a homolog and/or variant thereof as described herein.

In certain embodiments, mutation(s) are introduced into one or more COP1 nucleic acid sequence(s) of SEQ ID NO:11 or a homolog and/or variant thereof as described herein. In certain embodiments, mutation(s) are introduced into a nucleic acid sequence encoding one or more COP amino acid sequence of SEQ ID NO: 12 or a homolog and/or variant thereof as described herein.

In certain embodiments, mutation(s) are introduced into one or more CRY1 nucleic acid sequence(s) of SEQ ID NO:13 or a homolog and/or variant thereof as described herein. In certain embodiments, mutation(s) are introduced into a nucleic acid sequence encoding one or more CRY1 amino acid sequence of SEQ ID NO: 14 or a homolog and/or variant thereof as described herein.

In certain embodiments, mutation(s) are introduced into one or more HY5 nucleic acid sequence(s) of SEQ ID NO:15 or a homolog and/or variant thereof as described herein. In certain embodiments, mutation(s) are introduced into a nucleic acid sequence encoding one or more HY5 amino acid sequence of SEQ ID NO: 16 or a homolog and/or variant thereof.

In certain embodiments, mutation(s) are introduced into one or more PIF4 nucleic acid sequence(s) of SEQ ID NO:17 or a homolog and/or variant thereof as described herein. In certain embodiments, mutation(s) are introduced into a nucleic acid sequence encoding one or more PIF4 amino acid sequence of SEQ ID NO: 18 or a homolog and/or variant thereof.

In certain embodiments, mutation(s) are introduced into one or more PHYB nucleic acid sequence(s) of SEQ ID NO:19 or a homolog and/or variant thereof as described herein. In certain embodiments, mutation(s) are introduced into a nucleic acid sequence encoding one or more PHYB amino acid sequence of SEQ ID NO: 20 or a homolog and/or variant thereof.

In certain embodiments, the mutation(s) are loss of function mutations such as insertions, deletions or substitutions (e.g. single nucleotide polymorphisms) within the target gene(s) (e.g. SPA genes including one or more of SPA 1, 2, 3 and/or 4, BBX32 and/or COP).

In certain embodiments, a mutation may be introduced in the C-terminus of the target gene(s) (e.g. SPA genes including one or more of SPA 1, 2, 3 and/or 4, BBX32 and/or COP).

In certain embodiments, a mutation may be introduced in the N-terminus of the target gene(s) (e.g. SPA genes including one or more of SPA 1, 2, 3 and/or 4, BBX32 and/or COP).

In certain embodiments, one or more mutations may be “missense” mutations. A missense mutation is a change in the nucleic acid sequence that results in the substitution of an amino acid for another amino acid.

In certain embodiments, one or more mutations are “nonsense” mutations. A nonsense (or “STOP codon”) mutation is a change in the nucleic acid sequence that results in the introduction of a premature STOP codon and, thus, the termination of translation (resulting in a truncated protein). For example, any nucleotide substitution, insertion or deletion resulting in a “TGA” (“UGA” in RNA), “TAA” (“UAA” in RNA) or “TAG” (“UAG” in RNA) codon in the reading frame of the mature mRNA being translated will terminate translation.

In certain embodiments, one or more mutations are “insertion” mutations. An insertion mutation results from one or more nucleotides being added to the nucleic acid sequence.

In certain embodiments, one or more mutations are “deletion” mutations. A deletion mutation results from one or more nucleotides being deleted from the nucleic acid sequence.

In certain embodiments, one or more mutations are “frameshift” mutations. A frameshift mutation results from the nucleic acid sequence being translated in a different frame downstream of the mutation. A frameshift mutation can have various causes, such as the insertion or deletion of one or more nucleotides.

In certain embodiments, one or more mutations are “splice-site” mutations. A splice-site mutation results from the insertion, deletion or substitution of a nucleotide at the site of splicing.

In certain embodiments, one or more mutations are in a non-coding regulatory sequence (e.g. in a promoter of SPA genes including one or more of SPA 1, 2, 3 and/or 4, BBX32 and/or COP). A mutation in a regulatory sequence may be a change of one or more nucleotides compared to the wild type sequence, e.g. by replacement, deletion or insertion of one or more nucleotides. For example, such a mutation may lead to reduced or no mRNA transcript of the gene being made.

In certain embodiments, the mutation may result in a deletion of one or more nucleic acids (e.g. deletion of about 10, 20, 30, 50, 100 or more nucleic acids) from any one or more of SEQ ID NO: 1, 3, 5, 7, 9, 11, 13, 15, 17 or 19. In certain embodiments, the mutation may result in a deletion of amino acids (e.g. deletion of about 10, 20, 30, 50, 100 or more amino acids) from any one or more of SEQ ID NO: 2, 3, 6, 8, 10, 12, 14, 16, 18 or 20.

In certain embodiments, the mutation may result in an insertion of one or more nucleic acids (e.g. insertion of about 10, 20, 30, 50, 100 or more nucleic acids) from any one or more of SEQ ID NO: 1, 3, 5, 7, 9, 11, 13, 15, 17 or 19. In certain embodiments, the mutation may result in an insertion of amino acids (e.g. deletion of about 10, 20, 30, 50, 100 or more amino acids) from any one or more of SEQ ID NO: 2, 3, 6, 8, 10, 12, 14, 16, 18 or 20.

In certain embodiments, the mutation(s) are gain of function mutations within the target gene(s) (e.g. CRY1, HY5, PIF, PHYB).

In certain embodiments, gain of function mutations may result from one or more mutations in a coding sequence leading to constitutive activation of the resulting protein or by mutations that alter the level or pattern of gene expression. The latter type of mutations may be the result of altered promoter function in terms of the level of expression, for example, a constitutive versus inducible promoter, tissue or developmental stage specificity of a promoter or other regulatory element or enhanced native promoter activity.

In certain embodiments, gain of function mutations are produced by an insertional mutagen, e.g. comprising an enhancer element, followed by expression from the enhancer element. As used herein, “enhancer” and “enhancer element” are used interchangeably to refer to a nucleic acid sequence that functions to activate transcription of sequences from a nearby promoter. A promoter refers to a nucleic acid sequence that functions to direct transcription of downstream sequences. Sometimes, a promoter may function as an enhancer element.

In certain embodiments, the mutation (e.g. loss of function or gain of function) is introduced using mutagenesis (e.g. directed mutagenesis). Methods for mutagenesis in plants are well known in the art. Such technical processes lead to plants of the invention having modified genetic characteristics.

In certain embodiments, the mutagenesis is physical mutagenesis. For example, ultraviolet radiation, X-rays, gamma rays, fast or thermal neutrons or protons may be applied to the plant or seed thereof. The targeted population can then be screened to identify the loss of function (or gain of function) mutant.

In certain embodiments, the mutagenesis is chemical mutagenesis. For example, the chemical may be ethyl methanesulfonate (EMS), methylmethane sulfonate (MMS), N-ethyl-N-nitrosurea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7, 12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde.

In certain embodiments, the mutation is introduced using insertional mutagenesis. For example, transposons, site-directed nucleases (SDNs) or T-DNA mutagenesis may be used to reduce or abolish target gene expression and/or activity (e.g. SPA 1, 2, 3 and/or 4 expression and/or activity and/or other targets described herein).

In certain embodiments, the mutation is introduced by genome editing. Methods for genome editing in plants are well known in the art.

Any suitable method of genome editing may be used. For example, zinc finger nucleases (ZFNs), transcription activator-like effector nucleases (TALENs) or homing meganucleases may be used to edit the nucleic acid sequence encoding the target polypeptide (e.g. SPA 1, 2, 3 and/or 4 and/or other targets as described herein).

In certain embodiments, one or more nucleases from the bacterial adaptive immune system (e.g. Type II) are used to edit the nucleic acid sequence encoding the target polypeptide (e.g. SPA 1, 2, 3 and/or 4 and/or other targets as described herein). For example, CRISPR-associated proteins (e.g. Cas9, Cpf1, C2c1, C2c2, C2c3, Cas3, Cas3-HD, Cas 5, Cas7, Cas8, Cas10, or combinations or complexes thereof) may be used. In such embodiments, guide sequences may be used to target the nucleic acid sequence encoding the relevant polypeptide (e.g. SPA 1, 2, 3 and/or 4 and/or other targets as described herein). The use of CRISPR gene-editing technology in genome editing is well described in the art.

In certain embodiments, a crRNA (CRISPR RNA) and tracrRNA (trans-encoding CRISPR RNA) is used to guide the Cas endonuclease (e.g. Cas9) to at least one nucleic acid encoding the DNA target (e.g. SPA 1, 2, 3 and/or 4 and/or other targets as described herein). Typically, the crRNA contains a spacer region complementary to one strand of the double strand DNA target and a region that base pairs with the tracrRNA (trans-encoding CRISPR RNA) forming a RNA duplex that directs the Cas endonuclease to cleave the DNA target.

In certain embodiments, a single guide RNA (sgRNA) is used to guide the Cas endonuclease (e.g. Cas9) to at least one or more nucleic acids encoding SPA genes. sgRNA is a synthetic RNA chimera created by fusing crRNA with tracrRNA. The sgRNA guide sequence located at its 5′ end may confer DNA target specificity. In plants, sgRNAs have been expressed using plant RNA polymerase III promoters, such as U6 and U3. Thus, sgRNA may be designed to target the nucleic acid sequence encoding the target polypeptide (e.g. SPA 1, 2, 3 and/or 4 and/or other targets as described herein).

In certain embodiments, the mutation is identified by a targeting induced local lesions in genomes (TILLING) method. TILLING has been applied in many plant species, including tomato, rice, Arabidopsis, Brassica, maize and wheat. TILLING populations in crops are commercially available.

In certain embodiments, a mutation in a natural population is identified by EcoTILLING (see Till et al. 2006 (Nat Protoc 1: 2465-77) and Comai et al. 2004 (Plant J 37: 778-86). In certain embodiments, a TILLING method as described herein excludes EcoTILLING.

In TILLING, seeds may be mutagenized with chemical mutagens such as EMS. The resulting M1 plants may be self-fertilized and the M2 generation of individuals used to prepare DNA samples for mutational screening. DNA samples may be pooled and arrayed on microtiter plates and subjected to gene specific PCR.

The PCR amplification products may be screened for mutations in the target gene (e.g. SPA 1, 2, 3 and/or 4 and/or other targets as described herein) using any method that identifies heteroduplexes between wild type and mutant genes. Suitable methods include, for example, denaturing high pressure liquid chromatography (dHPLC), constant denaturant capillary electrophoresis (CDCE), temperature gradient capillary electrophoresis (TGCE) or by fragmentation using chemical cleavage. Preferably the PCR amplification products are incubated with an endonuclease that preferentially cleaves mismatches in heteroduplexes between wild type and mutant sequences. Cleavage products may be electrophoresed using an automated sequencing gel apparatus, and gel images analyzed with the aid of a standard commercial image-processing program.

Any primer specific to the target nucleic acid sequence (e.g. SPA 1, 2, 3 and/or 4 and/or other targets as described herein) may be utilized to amplify the target nucleic acid sequence within the pooled DNA sample. Preferably, the primer is designed to amplify the regions of the target gene where useful mutations are most likely to arise, specifically in the areas of the target gene that are highly conserved and/or confer activity. To facilitate detection of PCR products on a gel, the PCR primer may be labelled using any conventional labelling method.

In certain embodiments, rapid high-throughput screening procedures may be used to identifying one or more mutations in the target gene (e.g. SPA 1, 2, 3 and/or 4 and/or other targets as described herein). Once a mutation is identified, the seeds of the M2 plant carrying that mutation may be grown into adult M3 plants and screened for the phenotypic characteristics associated with loss of function of the target gene (e.g. increased photosynthetic capacity).

In the embodiments described above, one or more mutations are introduced by genetic engineering techniques. The plants (or parts or seed thereof) are not exclusively obtained by essentially biological processes, e.g. solely based on generating plants by traditional breeding methods.

The target polypeptide may be specifically localized in guard cells (e.g. SPA 1, 2, 3 and/or 4 and/or other targets as described herein). Advantageously, genetic engineering techniques such as those described above (e.g. TILLING) may therefore achieve tissue specific response without requiring the use of transgenes.

In certain embodiments, the expression of the target gene (e.g. SPA 1, 2, 3 and/or 4 and/or other targets as described herein) and/or activity of the target protein (e.g. SPA 1, 2, 3 and/or 4 and/or other targets as described herein) may be reduced or abolished at the level of transcription or translation. For example, expression of SPA nucleic acids can be reduced or abolished using gene silencing methods. “Silencing” refers to a down-regulation or complete inhibition of gene expression of the target gene or gene family. Methods for gene silencing in plants are well known in the art.

In certain embodiments, RNA interference, miRNA suppression, sense suppression, antisense suppression, virus-induced gene silencing or ribozymes are used to reduce or abolish the expression of at least one nucleic acid encoding the target polypeptide (e.g. SPA 1, 2, 3 and/or 4 and/or other targets as described herein).

In certain embodiments, short interfering RNA (siRNA), double-stranded RNA (dsRNA), micro-RNA (miRNA), antagomirs or short hairpin RNA (shRNA) capable of mediating RNA interference are used to reduce or abolish the expression of at least one nucleic acid encoding the target polypeptide (e.g. SPA 1, 2, 3 and/or 4 and/or other targets as described herein). The inhibition of expression of the target gene and/or target protein activity can be measured by determining the presence and/or amount of the relevant transcript using techniques well known to the skilled person (e.g., Northern Blotting, RT-PCR or the like).

In one embodiment, RNA interference (RNAi) is used to reduce transcript levels of the target gene (e.g. SPA 1, 2, 3 and/or 4 and/or other targets as described herein) in the plant. The technique of RNAi in plants is well described in the art.

In one embodiment, antisense RNA is used to reduce transcript levels of the target gene (e.g. SPA 1, 2, 3 and/or 4 and/or other targets as described herein) in the plant. In this method, RNA silencing does not affect the transcription of the target gene, but only causes sequence-specific degradation of target mRNAs. An “antisense” nucleic acid sequence comprises a nucleotide sequence that is complementary to at least a portion of the “sense” nucleic acid sequence encoding the target polypeptide. The use of antisense RNAi in plant gene silencing is well described in the art.

In one embodiment, artificial and/or natural microRNAs (miRNAs) may be used to reduce or abolish target gene transcription (e.g. SPA 1, 2, 3 and/or 4 and/or other targets as described herein) and/or mRNA translation. MicroRNAs (miRNAs) miRNAs are typically single stranded small RNAs typically 19-24 nucleotides long. Artificial microRNA (amiRNA) technology has been applied in plants to efficiently silence target genes of interest. The design principles for amiRNAs have been generalized and integrated into a Web-based tool (http://wmd.weigelworld.org). The use of miRNAs in plant gene silencing is well described in the art.

In one embodiment, a transgene is used to reduce or abolish the expression of the target gene (e.g. SPA 1, 2, 3 and/or 4 and/or other targets as described herein) and/or activity of the target protein (e.g. SPA 1, 2, 3 and/or 4 and/or other targets as described herein). Typically, the transgene has a similar sequence to the target gene. This sequence homology may involve promoter regions or coding regions of the target gene. When coding regions are involved, the transgene may have been constructed with a promoter that would transcribe either the sense or the antisense orientation of the coding sequence RNA.

In certain embodiments, the RNA or co-suppression molecule comprises a fragment of least about 17, 18, 19, 20, 25, 30 or 35 nucleotides (e.g. about 22 to 26 nucleotides) based on any nucleic acid sequence described herein (e.g. SEQ ID NOs 1, 3, 5, 7, 9 or 11 or variants thereof).

Guidelines for designing effective siRNAs are known to the skilled person. In preferred embodiments, the criteria for choosing a sequence fragment from the target gene mRNA to be a candidate siRNA molecule may include one or more of: (i) a sequence from the target gene mRNA that is at least about 50-100 nucleotides from the 5′ or 3′ end of the native mRNA molecule, (ii) a sequence from the target gene mRNA that has a G/C content of between about 30% and 70%, (iii) a sequence from the target gene mRNA that does not contain repetitive sequences, (iv) a sequence from the target gene mRNA that is accessible in the mRNA, (v) a sequence from the target gene mRNA that is unique to the target gene (or target gene family), (v) a sequence that avoids regions within 75 bases of a start codon. Software prediction programs are available that design optimal oligonucleotides (e.g. dsRNA) based on the above criteria. The optimized oligonucleotides may then be chemically synthesized and provided by commercial suppliers.

In certain embodiments, the RNA or co-suppression molecule (e.g. transgene) is introduced into the plant using conventional methods. Transformation methods in plants are well described in the art. For example, the RNA or co-suppression molecule may be introduced into the plant using a vector and/or Agrobacterium-mediated transformation. Stably transformed plants may be generated and the expression of the target gene (e.g. SPA 1, 2, 3 and/or 4 and/or other targets as described herein) compared to wild type or control plants.

In certain embodiments, the method comprises reducing or abolishing target protein activity (e.g. SPA 1, 2, 3 and/or 4 and/or other targets as described herein) in the plant. For example, antibodies or aptamers may be raised against the polypeptide. Such antibodies or aptamers, may, for example, reduce or abolish SPA activity through disrupting protein signaling or in other ways. Thus, antibodies or aptamers may be used to reduce or abolish SPA polypeptide activity in guard cells of plants. The raising of antibodies or aptamers against target polypeptides in plants is well described in the art.

Plants with Increased Photosynthetic Capacity

In certain embodiments, the invention provides plants having increased photosynthetic capacity. Typically, the PSII operating efficiency (Fq′/Fm′) is increased by at least about 10%, 20%, 30%, 40%, 50%, 60%, 70%, 80%, 90%, 100% or more as compared to a control or wild-type plant, wherein the actinic PFFD is about 800 μmol m⁻² s⁻¹. Preferably, the PSII operating efficiency is increased by at least about 40% or more as compared to a control or wild-type plant, wherein the actinic PFFD is about 800 μmol m⁻² s⁻¹.

Advantageously, the plants of the invention may have increased fitness to a changing environment as compared to control or wild type plants. In addition or alternatively, the plants of the invention may be grown more efficiently under controlled conditions (e.g. greenhouse) as compared to control or wild type plants.

In preferred embodiments, the plants have reduced or abolished expression of one or more nucleic acid encoding a SPA polypeptide and/or reduced or abolished activity of a SPA polypeptide (e.g. SPA1, SPA2, SPA3 and/or SPA4).

In certain embodiments, the plant has reduced or abolished expression of at least one nucleic acid encoding a BBX32 and/or COP polypeptide and/or reduced or abolished activity of a BBX32 and/or COP polypeptide.

In certain embodiments, the plant has increased expression of at least one nucleic acid sequence encoding CRY, HY5, PIF and/or PHYB polypeptide and/or increased activity of a CRY, HY5, PIF and/or PHYB polypeptide in the plant.

The plant may be any plant (or part thereof) as described herein. Preferably, the plant is a crop plant. Typically, the crop plant is grown under controlled light conditions (e.g. in a greenhouse).

In certain embodiments, the plant is genetically engineered or modified as described herein. Typically, the plant is not exclusively obtained by an essentially biological process.

In certain embodiments, the plant is genetically altered compared to a naturally occurring wild type plant. For example, the plant may have been altered compared to a wild type plant using any mutagenesis method described herein.

In certain embodiments, the plant is transgenic. For example, the plant may comprise a transgenic construct. The transgenic construct may reduce or abolish the expression of at least one nucleic acid encoding the target polypeptide (e.g. SPA1, 2, 3 and/or 4 or other targets as described herein). For example, the transgenic construct may lead to RNA interference, sense suppression, antisense suppression and/or miRNA suppression as described herein.

In certain embodiments, the plant has increased photosynthetic capacity as compared to a control or wild type plant as described herein.

In certain embodiments, shoot growth of the plant is significantly the same as compared to a control or wild type plant as described herein.

In certain embodiments, the plant has increased, biomass and/or yield as compared to a control or wild type plant described herein.

In certain embodiments, one or more mutations are introduced into the nucleic acid sequence encoding the target polypeptide (e.g. SPA1, 2, 3 and/or 4 or other targets as described herein).

For example, one or more mutation(s) may be introduced into coding or non-coding regulatory sequence of the target gene (e.g. SPA1, 2, 3 and/or 4 or other targets described herein).

In certain embodiments, one or more mutations are introduced into a least one plant cell and the plant is regenerated from the at least one mutated plant cell.

In certain embodiments, the invention provides a plant (or part thereof) obtainable by any method described herein.

In certain embodiments, the plant comprises one or more transgenic constructs. For example, the plant may comprise a construct (or vector) expressing RNA or a co-suppression molecule (e.g. transgene) that targets SPA expression as described herein. Typically, the construct is stably incorporated into the plant genome.

In certain embodiments, the plant is or has been selected for further propagation. The selected plants may be propagated by any suitable technique such as clonal propagation or classical breeding techniques. For example, a first generation (or T1) transformed plant may be selfed and homozygous second-generation (or T2) transformants selected. The T2 plants may then further be propagated through classical breeding techniques.

In certain embodiments, the invention provides a harvestable part of the plant (e.g. seed). Typically, the harvestable part of the plant (e.g. seed) comprises a mutation or transgenic construct as described herein.

Methods of Identifying Alleles Associated with Increased Photosynthetic Capacity

In certain embodiments, the invention provides a method of identifying one or more alleles associated with increased photosynthetic capacity in one or more plants.

In certain embodiments, the invention provides a method of screening a population of plants.

In certain embodiments, the method comprises detecting in the plant(s) one or more polymorphism(s) in a nucleic acid sequence encoding a SPA, BBX32 and/or COP polypeptide, wherein the one or more polymorphism(s) are associated with increased photosynthetic capacity. Preferably, the method comprises detecting in the plant one or more polymorphism in a nucleic acid sequence encoding a SPA polypeptide (e.g. SPA1, 2, 3 and/or 4).

In certain embodiments, the nucleic acid sequence is a genomic region encoding the target polypeptide or promoter regulating expression of the target polypeptide.

In certain embodiments, the method further comprises identifying one or more allele(s) at the one or more polymorphism(s) associated with increased photosynthetic capacity.

In certain embodiments, the invention provides a method of identifying (e.g. selecting) a plant having (or that will have) reduced or abolished expression of at least one nucleic acid encoding a SPA, BBX32 and/or COP polypeptide and/or reduced or abolished activity of a SPA, BBX32 and/or COP polypeptide as described herein.

In certain embodiments, the invention provides a method of identifying a plant having increased photosynthetic capacity and/or increased yield or biomass. For example, the one or more allele(s) may be used for marker-assisted selection of such plants.

Typically, the invention provides a method of detecting in the plant at least one marker that is indicative of reduced or abolished SPA expression. For example, this marker may be a SNP or polymorphism in a nucleic acid sequence encoding a SPA polypeptide as described herein. Alternatively, the marker may be a polymorphism at a highly associated marker locus, wherein the sequence at this locus is indicative of reduced or abolished SPA expression (e.g. a linked marker). Typically, a linked marker is within about 10 cM, 5 cM, 4 cM, 3 cM, 2 cM, 1 cM or less from the causative mutation.

In certain embodiments, the invention provides a method for selecting plants having increased photosynthetic capacity by screening for the presence of any one or more marker(s) as described herein.

In certain embodiments, the invention provides use of any one or more marker(s) as described herein to select a plant having increased photosynthetic capacity.

In certain embodiment, the plant is a polyploid. In such embodiments, the marker may be identified in one, two or more copies of the genome.

Suitable tests for assessing the presence of a polymorphism are well known to the skilled person, and include but are not limited to, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs). In one embodiment, Kompetitive Allele Specific PCR (KASP) genotyping is used.

In certain embodiments, the method may comprise obtaining a nucleic acid sample from the one or more plants. Such methods may further comprise carrying out amplification of the nucleic acid sequence encoding the target polypeptide (e.g. SPA 1, 2, 3 and/or 4 or other targets described herein).

In certain embodiments, the nucleic acid sequence encoding the target polypeptide (e.g. SPA 1, 2, 3 and/or 4 or other targets described herein) is amplified by PCR.

In certain embodiments, the method may comprise introgressing the nucleic acid sequence comprising the one or more polymorphism(s) into a second plant. For example, the method may comprise introgressing the chromosomal region comprising the one or more polymorphism(s) into a different genetic background (e.g. an improved crop variety).

### High Throughput Screening of Plants

In certain embodiments, the invention provides a method of quantifying photosynthetic capacity in one or more plants. Typically, the method comprises measuring photosystem II (PSI I) operating efficiency, linear electron flux and/or quantum yield of CO₂ assimilation in the one or more plants. The one or more plants (e.g. population of plants) may include any plant as described herein.

Typically, the invention provides a method of quantifying photosynthetic capacity in one or more plants having modified CRY1-directed signaling (e.g. reduced or abolished expression of at least one nucleic acid sequence encoding a SPA polypeptide and/or reduced or abolished activity of a SPA polypeptide). Typically, the photosynthetic capacity is compared to a control or wild type plant as described herein.

Any suitable measurement may be taken to quantify the photosynthetic capacity of the one or more plants as described herein. Preferably, chlorophyll fluorescence (CF) is used to determine photosynthetic capacity.

In certain embodiments, plants are subjected to high light conditions, e.g. a PPFD of about 1100 μmol m⁻² s⁻² (e.g. between 1000 to 1200 μmol m⁻² s⁻²) typically for about 4 hours. The plants may then be subjected to low light conditions, e.g. a PPFD of about 100 μmol m⁻² s⁻² (e.g. between 90 to 110 μmol m⁻² s⁻²) typically for about 0.5 hours. The plants may then be exposed to a range of actinic PPFDs (e.g. for about 50 minutes) to collect CF measurements. For example, plants may be exposed to increasing actinic PPFD from about 200-to about 1400 μmol m⁻² s⁻² in about 200 μmol m⁻² s⁻² steps about every 5 min as described above. Typically, measurements are taken over 1 to 5 days (e.g. at day 1 and/or 5 as described elsewhere herein).

In certain embodiments, the PSII operating efficiency (Fq′/Fm′) is increased by about 40% or more as compared to a control or wild-type plant. Typically, the PSII operating efficiency is determined at a PFFD of about 800 μmol m⁻² s⁻¹ as described elsewhere herein.

In certain embodiments, the method of quantifying photosynthetic capacity in one or more plants further comprises determining the minimal number of genes in the CRY1-dependent signaling pathway to modulate in order to achieve increased photosynthetic capacity whilst avoiding or reducing adverse effects on plant growth.

For example, the methods described herein may determine the minimal number of SPA genes to target in order to increase PSII operating efficiency (Fq′/Fm′) by about 40% or more as compared to a control or wild-type plant. If targeting a first SPA gene in the plant leads to an increase in photosynthetic capacity of less than about 40%, then the expression and/or activity of one or more additional SPA genes (and/or other targets described herein) may be modulated until an increase in photosynthetic capacity of about 40% or more is achieved.

### Methods of Producing Food or Feed Products Under Controlled Light Conditions

In certain embodiments, the invention provides a method of producing a food or feed product in a plant having modulated CRY1-directed signaling as described herein, wherein the plant in grown under controlled light conditions. For example, the method may comprise reducing or abolishing the expression of at least one nucleic acid encoding a SPA polypeptide and/or reducing or abolishing the activity of a SPA polypeptide according to any method described herein.

In preferred embodiments, the controlled lighting conditions are optimized (e.g. the duration and/or intensity of lighting is adjusted) depending on the maximal photosynthetic capacity of the plants.

In certain embodiments, the method comprises producing a plant with reduced or abolished expression of at least one nucleic acid encoding a SPA polypeptide and/or reduced or abolished activity of a SPA polypeptide according to any method described herein.

In certain embodiments, the method comprises obtaining a plant part from the plant.

In certain embodiments, the method comprises producing a food or feed product from the plant part.

In certain embodiments, the invention provides use any plant or plant part as described herein to produce a food or feed product.

In certain embodiments, the food or feed product is non-propagation material such as flour, oil, fatty acids or the like.

In certain embodiments, the plant part is a seed.

In certain embodiments, the invention provides a seed produced from a genetically engineered or transgenic plant as described herein.

In the following, the invention will be explained in more detail by means of non-limiting examples of specific embodiments.

### EXAMPLES

The photosynthetic capacity of mature leaves increases after several days' exposure to constant or intermittent episodes of high light (HL) and is manifested primarily as changes in chloroplast physiology. This is termed dynamic acclimation but how it is initiated and controlled is unknown. From fully expanded Arabidopsis leaves, HL-dependent changes in transcript abundance of 3844 genes in a 0-6 h time-series transcriptomics experiment was determined.

It was hypothesised that among such genes were those that contribute to the initiation of dynamic acclimation. By focusing on HL differentially expressed transcription (co-)factor (TF) genes and applying dynamic modelling to the temporal transcriptomics data, a gene regulatory network (GRN) of 47 predominantly photoreceptor-regulated (co)-TF genes was inferred. The most connected gene in this network was B-BOX DOMAIN CONTAINING PROTEIN32 (BBX32). Plants over-expressing BBX32 were strongly impaired in dynamic acclimation and displayed perturbed expression of genes involved in its initiation. These observations led to demonstrating that as well as regulation of dynamic acclimation by BBX32, CRYPTOCHROME1, LONG HYPOCOTYL5, CONSTITUTIVELY PHOTOMORPHOGENIC1 and SUPPRESSOR OF PHYA-105 are also important regulators of this process. Additionally, the BBX32-centric GRN provides a view of the transcriptional control of dynamic acclimation distinct from other photoreceptor-regulated processes, such as seedling photomorphogenesis.

It was hypothesised that the first hours of exposure of mature leaves to HL initiates dynamic acclimation although an increase in photosynthetic capacity takes several days to develop. This hypothesis is an extension of an earlier proposal regarding the temporal order of events leading to protection against oxidative stress induced photoinhibition, the re-structuring of light harvesting antennae and PSI and PSII reaction centers (Eberhard et al., 2008). This hypothesis was tested by first developing HL and low light (LL) time series transcriptomics datasets from a defined fully expanded leaf of LL-grown Arabidopsis plants. Analysis of these data revealed that processes interpreted as the first steps in establishing dynamic acclimation could be identified and were largely complete within 4 h of HL exposure. From comparison with other published transcriptomics datasets, an over representation of differentially expressed transcription (co)factor genes involved in light- and photoreceptor-regulated photomorphogenesis of seedlings was observed. This led, via dynamic modelling, to the identification of B-BOX DOMAIN CONTAINING PROTEIN32 (BBX32) as a negative regulator of dynamic acclimation. BBX32 encodes a Zn-finger transcription co-factor which forms associations with diverse transcription regulators, such as LONG HYPOCOTYL5 (HY5) and BBX4 to alter their function and is most studied in the context of seedling photomorphogenesis and the control of flowering time (Holtan et al., 2011; Park et al., 2011; Gangappa and Botto, 2014; Tripathy et al., 2017). In turn, the involvement of BBX32 led to the identification of HY5, CRY1, PHYTOCHROME INTERACTING FACTOR (PIF) genes and COP1/SUPPRESSOR OF PhytochromeA-105 (SPA) genes as further regulators of dynamic acclimation. Thus, many signalling aspects of photoreceptor-driven seedling photomorphogenesis are also active in mature fully expanded leaves to control dynamic acclimation.

### Example 1—GO Analysis of Time Series Transcriptomics of HL-Exposed Leaves Provides Insights into the Initiation of Dynamic Acclimation

The starting point for this study was the development of a HL time series transcriptomics experiment. Groups of time-resolved differentially expressed genes (DEGs) were subjected to Variational Bayesian State Space Modelling (VBSSM; see Methods), which requires a determination of steady state transcript levels at a minimum of 12 evenly-spaced time points (Beal et al., 2005; Penfold and Wild, 2011; Penfold and Buchanan-Wollaston, 2014; Bechtold et al., 2016). Therefore, 30 min sampling over a 6 h HL period beginning 1 h after subjective dawn was chosen (see Methods). This time period was chosen because it spans the initiation of both the short term and long-term acclimation to HL proposed by Eberhard et al. 2008.

Full transcriptome profiles using CATMA microarrays (Sclep et al., 2007) were obtained from leaf 7 of HL-exposed plants along with parallel LL controls (see Methods). Microarray analysis of variance (MAANOVA; Wu et al., 2003; see Methods) was used to extract expression values from each probe for every treatment for each technical and biological replicate. To determine DEGs that showed a significant difference between HL-exposed leaves and the LL controls over all or part of the time period, a Gaussian process two-sample test (GP2S; Stegle et al., 2010) was applied and 4069 probes were selected with a Bayes factor score >10 which corresponded to 3844 DEGs. The full data set is deposited with Gene Expression Omnibus (GEO; GSE78251).

To obtain further insight into the overall response to HL at the molecular level, hierarchical co-cluster analysis of the 3844 DEGs was carried out using SplineCluster (Heard et al., 2005). It was reasoned that groups of DEGs that display similar temporal patterns of expression could be co-regulated and clustering would be useful in identifying groups of genes for dynamic modelling. On the basis of comparing temporal gene expression patterns in both the HL-exposed and control LL leaves (see Methods), the 3844 DEGs were divided into 43 temporal clusters (FIG. 1A). The outcome of this co-clustering was differential transcript abundance between LL and HL conditions superimposed on a range of temporal expression trends across the diel.

The clusters are ordered such that 1-13 show general transcript abundance to be lower in HL vs LL samples and/or displayed a downward pattern over the diel (FIG. 1A). This pattern changes progressively with increasing degree of expression being higher in HL than LL but against a descending diel pattern in clusters 14-20 (FIG. 1A), followed by transient but progressively increasingly greater differential transcript levels in HL samples compared with LL in clusters 21-26 (FIG. 1A) to progressively sustained periods of higher expression in HL compared with LL from cluster 27-43 against a background of level or increasing transcript levels across the diel (FIG. 1A).

To gain a better view of the timings of differential expression in response to HL, the DEGs from the time-local GP2S were used to identify intervals of differential expression as described by Windram et al. (2012). A histogram of the time of first differential expression (HL compared to LL) is shown in FIG. 1B and indicates that response to HL is rapid with >700 genes becoming differentially expressed by 1 hour into the HL time course. Nevertheless, it was also clear that changes in transcript abundance were being initiated for significant numbers of genes up to 4 h HL. In summary, the response of the leaf 7 transcriptome to HL entails changed expression in response to the stimulus, with changes occurring across the time of the experiment against a backdrop of complex changes in transcript abundance across 6 h out of an 8 h photoperiod.

Gene Ontology (GO) analysis showed that clusters 22, 23, 25 were highly enriched for generic abiotic stress-defensive genes (P value <0.1, Bonferroni corrected). In contrast, some of the other clusters displayed a different set of GO function enrichments (data not shown). These multiple enriched sets were consistent with a re-adjustment to cellular metabolism. For example, in clusters 39 and 41-43 with generally higher expression in HL compared with LL, there was over-representation of genes associated with amino acid and protein synthesis respectively. Among the clusters showing a lowered expression in HL compared with LL, there was enrichment for genes associated with cell wall metabolism (callose deposition, cell wall thickening Cluster 1), phenylpropanoid and glucosinolate metabolism (Clusters 1 and 10 respectively), basal resistance to infection (Cluster 3) and chromatin re-modelling (cluster 10).

### Example 2—Induction of Dynamic Acclimation

To test the interpretation of the HL time series data, it was determined whether dynamic acclimation could be induced by exposing a plant every day to 4 h HL (see Methods). This period of HL exposure was chosen since most differential expression had been initiated by this time (FIG. 1B). Other than being shortened to 4 h, the environmental conditions were the same as for the time series transcriptomics experiment (see Methods). The daily HL regime brought about a stepwise increase in the operating efficiency of PSII (Fq′/Fm′) (Baker, 2008) of fully expanded leaves (FIG. 2A). By day 5, PSII operating efficiency had increased substantially (e.g. 78% at 800 μmol m-2 s-1 actinic PPFD; FIG. 2B; see also FIG. 4B). This pattern was followed by equivalent changes in Fv′/Fm′ and Fq′/Fv′. Fv′/Fm′ indicates the maximum operating efficiency of PSII at a given PPFD and a rise in this parameter indicates a decline in NPQ (Baker, 2008). Fq′/Fv′ is the PSII efficiency factor and is mathematically identical to the coefficient for PQ (qp) and indicates increased capacity to drive electron transport (Baker, 2008). Control LL-grown plants of the same age as the plants subjected to 5 daily HL treatments did not show these changes in chlorophyll fluorescence (CF) parameters. The first exposure to HL (day 1) did not result in irreversible photoinhibition or significant tissue damage. This was confirmed in the HL time series data, which used the same PPFDs, in which steady levels of transcripts for genes considered to be markers for H2O2 (APX2 and FER1, Ball et al., 2004; Gadjev et al., 2006) rose but those associated with ¹O2-induced signalling (AAA-ATPase and BAP1, Ramel et al., 2013) remained unchanged or declined. The changes in the expression of these marker genes indicated the HL treatment used in the time series transcriptomics experiments, also did not elicit photodamage and provided conditions that could promote dynamic acclimation.

The increased operating efficiency of PSII (Fq′/Fm′ and also Fq′/Fv′) after the 5-day HL treatment (FIG. 2A) could have reflected enhanced photosynthetic capacity. To test this possibility, gas-exchange measurements for photosynthesis were carried out (see Methods). The same experiment was repeated and, in the photoperiod following the last HL treatment, measurements of CO₂ assimilation rates (A) over a range of light intensities in fully expanded leaf 7 of these plants (Boyes et al., 1998) were taken. This showed that the light-saturated photosynthetic rate (Asat) was significantly greater (P<0.001) by 64% compared with LL control plants (FIG. 2C). In contrast, after a single 4 h HL exposure, followed by photosynthesis measurements in the next photoperiod, no increase in Asat was observed (FIG. 2C). In a separate series of experiments, the measurement of A over a range of internal leaf CO₂ concentrations (Ci) also showed that the maximal CO₂-saturated rate of photosynthesis (Amax) increased by 31% (P<0.002) after 5 daily HL exposures (FIG. 2D). This confirmed that repeated HL exposures did not solely affect stomatal behaviour but brought about an increase in foliar photosynthetic capacity. The changes in CF parameters by day 5 of HL treatments observed in the previous experiments (FIG. 2A) occurred also in larger older leaves that were required for gas exchange measurements.

In summary, increased Asat and Amax after 5 days of repeated HL exposure (FIG. 2C, D) was accompanied by a highly significant increase in Fq′/Fm′ (FIG. 2A; P<0.0001, ANOVA and Tukey HSD) reflecting an increased photochemical efficiency to support dynamic acclimation. Therefore, a substantial (>40%; typically using the median 800 μmol m-2 s-1 actinic PPFD value) change in Fq′/Fm′ between days 1 and 5 of repeated HL was subsequently used as a more convenient image-based measurement of the establishment of dynamic acclimation and consequent increased photosynthetic capacity.

### Example 3—Dynamic Modelling Infers a BBX32-Centric Network of HL-Regulated Transcription Factor Genes

The HL time series data were used to infer gene regulatory networks (GRNs) using VBSSM (Beal et al., 2005; Penfold and Wild 2011). VBSSM was chosen because it has been demonstrated to assemble known GRNs from temporal gene expression data and to infer novel GRNs whose highly connected genes (nodes) have subsequently been shown experimentally to have a novel and important function (Beal et al., 2005; Penfold and Wild, 2011; Breeze et al., 2011; Penfold and Buchanan-Wollaston, 2014; Windram and Denby 2015; Bechtold et al., 2016). However, with the number of time points used, only data from genes or probes could be modelled by VBSSM. This avoids over-fitting the data but constrains network size (Beal et al., 2005; Allahverdeyeva et al., 2015; Windram and Denby, 2015; Bechtold et al., 2016). To accommodate this limitation, DEGs coding for transcription (co-) factors (TFs) were focused on. It was reasoned that TF GRNs would control the expression of a wide network of genes and by inferring GRNs this would allow the most connected TF genes to be identified, often termed hub genes (Windram and Denby, 2015; Albihlal et al., 2018). Consequently, it was reasoned that hub TF genes would directly and indirectly regulate the expression of a sufficiently large number of genes to influence whole leaf HL responses and acclimation phenotypes. Therefore, the intention was to screen highly connected candidate hub TF genes directly for their impact upon whole plant dynamic acclimation.

It was estimated that there were 371 HL DEGs coding for TFs or transcription cofactors and therefore, it was necessary to apply further criteria for the selection of <100 HL TF DEGs for VBSSM. To further narrow the selection, comparisons were made between the 43 HL temporal clusters (FIG. 1A) and 14 publicly available transcriptomics data sets or meta-analyses of such data for treatments or mutants perturbed in chloroplast-to nucleus and ROS-mediated signalling. On a cluster-by cluster basis, the highest number of significant (P<0.00001) overlaps in clusters 1, 2, 3, 5, 6, 9, 10, 14, 16, 17 and 27 were encountered with phyA/phyB DEGs among which genes involved in chloroplast-to-nucleus (retrograde) signalling had been identified (Shikata et al., 2014). This observation suggested that photoreceptor-mediated regulation of HL-responsive genes was highly represented in the time series transcriptomics dataset. Therefore, it was examined whether photoreceptor-regulated TF and co-transcription factor genes (Shikata et al., 2014; Dong et al., 2014) were also over-represented in the HL dataset. This was the case. Ninety-one (91) photoreceptor- and light-regulated TF DEGs were over-represented in the time series transcriptomics data, irrespective of which temporal cluster they were drawn from (P=1.4E-06; Hypergeometric Distribution Test). The HL time series expression data from these 91 genes were used to infer VBSSM networks.

The first inferred network for HL revealed LATE ELONGATED HYPOCOTYL (LHY) as the most highly connected gene. However, mutant lhy-21 plants were not perturbed in dynamic acclimation. Therefore, the VBSSM modelling was re-iterated but omitting LHY expression data. This inferred a 47 node-HL network centred on BBX32 (FIG. 3). Further confirmation of the potential importance of BBX32 was also apparent when a network using the time series HL data from the same 91 (co)TF genes was inferred using Causal Structure Identification (CSI; Klemm 2008; Penfold and Wild 2011; Windram et al., 2012; see Methods). Again, LHY and BBX32 were the top two most connected genes, with 14 and 11 edges respectively.

### Example 4—BBX32 is a Negative Regulator of Dynamic Acclimation

Dynamic acclimation was tested in two independent BBX32 over-expressing BBX32-OE) genotypes (BBX32-10 and BBX32-12) and a T-DNA insertion mutant (bbx32-1; see Methods and Holtan et al., 2011). BBX32-OE plants showed a highly significant impairment of dynamic acclimation (FIG. 4A). In contrast, bbx32-1 plants showed a weak but significant accelerated dynamic acclimation phenotype (FIG. 4B). An accelerated acclimation phenotype is defined as a significant enhancement of PSII operating efficiency over one or more days in the 5d serial HL treatment. The strong negative impact of BBX32 over-expression on dynamic acclimation was confirmed subsequently by showing a significant inhibition of photosynthetic capacity (Asat) after 5 days of daily 4 h HL exposure (FIG. 4C). Consequently, it was concluded that BBX32 is a negative regulator of dynamic acclimation.

### Example 5—Transcriptomics Provides a Partial Verification of the BBX32 HL TF Network

In order to explore further the connections depicted in the network model (FIG. 3), massively parallel RNA sequencing (RNAseq) was carried out (see Methods; GEO; GSE158898) to profile the foliar transcriptome of fully expanded leaves of Col-0 and BBX32-OE plants exposed to 3.5 h HL in comparison with LL controls. From the RNAseq data, the transcript levels of 25 of 47 constituent genes in the inferred network were significantly altered by constitutive BBX32 over expression compared with Col-0 plants in LL and/or HL (FIG. 5). Therefore, these data partly validated this (co)TF GRN and suggested that BBX32 engages in both positive and negative regulation of the other TF genes in this network (FIG. 3; FIG. 5).

### Example 6—The Transcriptome of BBX32OE Plants Links Initial Responses to HL with Dynamic Acclimation

The greatly impaired ability of BBX32-OE plants to undergo dynamic acclimation (FIG. 4A, C), prompted an analysis of the RNAseq data on the impact of BBX32 over expression on the transcript levels of photosynthesis-associated genes (PhAGs; https://www.kegg.jp/dbget-bin/www_bget?pathway+ath00195). There was a clear influence of BBX32 over-expression under LL and HL on the transcript levels of a range of transcripts coding for LH Antenna proteins, Calvin-Benson cycle enzymes and components of photosynthetic electron transport, PSI and PSII (FIG. 6). It was concluded that these and other transcripts affected in BBX32-OE plants may reflect their perturbed photosynthetic physiology.

This widespread disruption of PhAG transcript levels led to examination of the impact of BBX32 over-expression on other cellular processes. In the RNAseq experiment, of the 2903 genes whose transcript levels were HL responsive (Padj.<0.05; >2-fold differentially expressed), BBX32 over-expression perturbed the transcript levels of 32% and 15% of them in LL and HL conditions respectively (FIG. 7A). The HL/LL Col-0 DEGs were enriched for 35 GO BP terms and 26 of them were also significantly over-represented in the BBX32-OE/Col-0 LL and BBX32-OE/Col-0 HL DEGs. These shared GO groups all describe responses to various abiotic and biotic stresses or response to endogenous stimuli such as salicylic acid or H2O2. This analysis indicates that BBX32 influences a wide range of cellular responses to stress, which includes regulation of genes associated with basal immunity to infection.

The DEGs from BBX32-OE HL and LL treated plants were also compared with the 3844 time-series HL DEGs (FIG. 7B). Although the number of overlapping genes was lower (FIG. 7B), BBX32-OE HL DEGs again confirmed enrichment for a range of GO terms that describe generic responses to environmental stress. However, the BBX32-OE LL DEGs also differentially expressed in the HL time series dataset, also revealed significant enrichment (FDR<0.05) of a range of additional functions including glucosinolate and glycosinolate metabolism (GO:0019760, GO:0050896, GO:006143, GO:0019757, GO:0016144, GO:0019761, GO:0019758), cell wall thickening (GO:0052543, GO:0052386) and callose deposition (GO:0052543, GO:0052545). Down-regulation of these groups of genes in the HL time series data may reflect a redistribution of resources towards dynamic acclimation and away from basal immunity suggest BBX32 may play a regulatory role in these processes (see Discussion) but also reinforces that BBX32 influences immediate responses before or during a single exposure to HL.

### Example 7—CRY1- and HY5-Regulated Control of Dynamic Acclimation

BBX32 has been proposed to be a negative regulator of the integration of light signals from phytochromes (PHYs) and cryptochromes (CRYs) during photomorphogenesis (Holtan et al., 2011; Gangappa and Botto, 2014). BBX32-OE seedlings display a long hypocotyl phenotype in the light like photoreceptor mutants and mutations in LONG HYPOCOTYL5 (HY5; Holtan et al., 2011). Notably, HY5 is a member of the BBX32-centric GRN (FIG. 3; FIG. 5) and along with CRY1, has also been implicated in influencing the expression of HL-inducible gene expression (Kleine et al., 2007; Shaikali et al., 2012; Chen et al., 2013). Furthermore, PHYA-, PHYB- and CRY1-mediated signaling was proposed to regulate maximum photosynthetic capacity in plants grown in a range of PPFDs (Walters et al 1999). Dynamic acclimation in photoreceptor-defective and hy5 mutants was therefore tested.

No significant impact of PHYA or PHYB on acclimation was observed. In contrast, cry1 mutants almost completely failed to undergo any dynamic acclimation (FIG. 8A, B), whereas cry2-1 was not impaired. One of the cry1 mutants shown (cry1M32; FIG. 8B) arose from a screening of T-DNA insertion mutants in genes coding for 7-transmembrane proteins that had been postulated to be implicated in HL-mediated G protein signaling (Galvez-Valdivieso et al., 2009; Gorecka et al., 2014). However, the one mutant recovered from this screening, was shown subsequently to be deficient in dynamic acclimation due to a disabling second site mutation in CRY1 (see Methods). Since the defective acclimation phenotype was identified prior to knowing the identity of the causal mutation this was taken as forward genetic evidence of the importance of CRY1 in dynamic acclimation in mature leaves.

The light environment used to grow plants for this study and subject to HL was enriched for blue wavelengths (see Discussion). Therefore, it was considered the possibility that a role for phytochromes in dynamic acclimation could be obscured, favouring a predominance of CRY1 under the growth conditions of the present study. To test this notion, a mutant harbouring a constitutively active form of PHYB, phyBY276H (YHB) in a Col-0 background (Jones et al., 2015) was tested for dynamic acclimation (FIG. 8C). This mutant exhibited a higher PSII operating efficiency than Col-0 after 1 day of HL exposure. This accelerated acclimation phenotype is in keeping with being a constitutively active positive regulator of dynamic acclimation.

Mutants defective in HY5 function were strongly impaired in dynamic acclimation (FIG. 8D, E) consistent with being a member both of a BBX32-centric GRN and being a positive regulator of CRY1-mediated dynamic acclimation (FIG. 8A, B).

### Example 8—COP1, PIF and SPA Genes Regulate Dynamic Acclimation

In both photomorphogenesis and shade avoidance responses, the transduction of signals from photoreceptors is mediated via one or more DET/COP/FUS regulatory complexes (Lau and Deng, 2012), which act as platforms for the post-translational control of the levels of HY5 and the integration into the signaling of the TFs PHYTOCHROME INTERACTING FACTORS (PIFs) and regulatory proteins SUPPRESSOR OF PHYA-105 (SPA) (Hardtke et al 2000; Toledo-Ortiz et al 2003; Lian et al 2011; Dong et al 2014; Huang et al 2014; Lau and Deng 2012; Gangappa and Botto, 2016; Hoecker 2017; Pham et al 2018; Lau et al., 2019). CRY1 and PIFs have been shown also to physically interact independent of COP/DET/FUS (Pedmale et al., 2016; Ma et al., 2016). In the VBSSM-inferred GRN, PIF4 and SPA1 were predicted to have a regulatory connection to BBX32 (FIG. 3; FIG. 5). Significantly, 187 HL time series DEGs overlapped (P=0.0018; Hypergeometric Distribution Test) with a set of 1120 genes identified as commonly regulated by SPA1, 2, 3, 4, PIF1, 3, 4, 5 and COP1 in de-etiolating and light-exposed seedlings (Pham et al., 2018). Interestingly, the most significant GO Biological Process function coded by these overlapping genes was Photosynthesis (GO: 0015979; FDR=2.7 E-17). Cop1-4 plants, despite a severely dwarfed shoot morphology (FIG. 9A; Deng and Quail, 1992; Gangappa and Kumar, 2018), displayed a highly elevated PSII operating efficiency (Fq′/Fm′) by day 1 of the HL acclimation regime compared with Col-0 (FIG. 9B) like the HL response of YHB plants (FIG. 8C). In contrast, despite a similar dwarf shoot morphology (FIG. 9A), det1-1 displayed no defect in dynamic acclimation (FIG. 9D). This strongly suggests that the dynamic acclimation response of chloroplasts is independent of shoot size and that these two traits are not coupled. Furthermore, spa1/spa2/spa3 (spa1,2,3) plants also displayed an accelerated acclimation phenotype (FIG. 9C). Therefore, it was concluded that one or more type of COP1/SPA complex (Huang et al., 2014; Hoecker, 2017) are negative regulators of dynamic acclimation and that DET1 plays no role in dynamic acclimation. There is a high degree of redundancy among the PIF TF family and therefore a quadruple null mutant of PIF1, 3, 4 and 5 (hereafter called pifq; Leivar et al., 2008) was tested for its capacity for dynamic acclimation. These plants displayed a severe dwarf phenotype as previously described (Leivar et al., 2008), but also a significant inhibition of dynamic acclimation (FIG. 9E). In contrast, the dynamic acclimation of a single mutant allele of PIF4 (pif4-2) was normal.

### Example 9—Isolation and Targeting of SPA Genes in Different Crop Species

There are 4 Arabidopsis SPA genes (Laubinger, S. et al 2004; Plant Cell 16: 2293). The nucleic acid sequences of these SPA genes or the corresponding amino acids are used to isolate SPA homologs from other crop species. Sequence alignment of SPA1, SPA2, SPA3 and SPA4 reveals the four proteins are closely related within A. thaliana (FIG. 11). The A. thaliana sequences are used in the NCBI—nr GENBANK sequence database to identify equivalent SPA genes in other species. Using all genome sequence databases as the source tend to give the best matches with Brassicaceae, which are related to Arabidopsis, but sequences are clearly seen for unrelated species, e.g. Quercus sp. (oak), cotton (Gossypium sp.) etc. FIG. 12 shows a phylogenetic tree diagram, which shows that SPA genes are highly conserved across the plant kingdom.

Many crop species are greater than diploid (e.g. wheat is hexaploid) meaning that multiple SPA genes are recovered in such species corresponding to only one in a diploid species such as Arabidopsis or tomato. In non-diploid plants, SPA gene expression is determined, and the necessary number of SPA genes targeted.

An Example output screening against Arabidopsis SPA1 (top pages—first 50 outcomes of alignments shown only) from screening all plant sequence database (NCBI—nr GENBANK) is shown in FIG. 14.

The same approach is used to identify conserved SPA2, 3 and 4, CRY1, PHY and COP1 genes in different species. The SPA protein sequences are next compared between Arabidopsis and tomato. FIG. 13 shows a phylogram tree of protein sequences alignments between the SPA isoforms in Arabidopsis and tomato.

This alignment shows more than one tomato SPA isoform for each Arabidopsis isoform. This could be due to multiple genes, although the sequence variation is slight, so this is unlikely. More likely this due to splice RNA variants or simple sequencing differences between multiple database entries or real but minor differences between cultivars. The two tomato sequences with closest homology to Arabidopsis SPA1 are set forth in SEQ ID NO: 21 and 22.

The DNA sequences for LsSPA genes are next recovered by several approaches: PCR, screening of libraries or most cost effectively, by having the genes synthesised. However, since SPA genes are negative regulators of photosynthetic capacity and dynamic acclimation, these sequences are used to develop a range of mutagenesis approaches to reduce or abolish expression and/or activity of the SPA genes in the tomato genome:

### Non-GM Approaches

1. Natural variants are screened in a collection of tomato germplasm. Genotypes (varieties, accessions) are identified with naturally occurring mutations that disable the SPA protein function or expression. The trait is then introgressed into elite crop germplasm using phenotypic screening for enhanced photosynthetic capacity and efficiency and/or marker-assisted selection using SPA gene sequences (or linked markers).
2. Mutation breeding I—random mutagenesis using mutagens such as X-rays, gamma rays, mutagenic chemicals such as ethylmethyl sulphonate is used to identify tomato spa mutants. Mutant plants are phenotypically and genetically screened as in 1.
3. Mutation breeding II—retrotransposon mutagenesis is used to identify tomato spa mutants.
4. A TILLING population is used to identify spa mutants in tomato using available TILLING populations (Minoia, S. et al (2010) BMC Res Notes 3, 69 https://doi.org/10.1186/1756-0500-3-69) Reverse genetics in TILLING uses a high throughput method to screen SPA gene sequences for mutations.

### GM Approaches

1. Directed mutagenesis or gene editing is used to engineer SPA mutations in crop species (e.g. tomato). Using CRISPR/Cas9, the SPA sequence in tomato (SEQ ID NO: 21 and/or 22) is used to design sg (guide) RNAs to cause deletion of the target genes. This method is particularly suitable where multiple SPA genes are deleted or disabled to achieve the desired outcome of enhanced photosynthetic capacity.
2. Antisense or RNAi methodology. The identified SPA homolog DNA sequences (SEQ ID NO: 21 and/or 22) are used to design inhibitory RNA transgenes as short RNAs (interfering RNAs) or longer antisense RNAs. These are designed to target more than one member of a gene family.

### Example 10—Tailoring Photosynthetic Capacity in Plants by Targeting One or More SPA Genes

Repeated exposure of spa1,2,3 triple mutant plants shows a substantial elevation in photosynthetic efficiency (Fq′/Fm′) irrespective of the number of sequential HL exposures (FIG. 15). Each time point represents a daily exposure of plant to 4 h high light (1000 μmol m⁻² s⁻¹) within an 8 h day (photoperiod) in which plants, if not exposed to high light) were grown at their growth light intensity (150 μmol m⁻² s⁻¹).

From FIGS. 15 and 16 it can be confirmed that disabling SPA genes in plants releases a degree of negative control on the plant achieving maximum photosynthetic capacity over a range of light conditions. The example provided disables 3 out of 4 SPA genes in this species. Importantly, it transpires that loss of any single SPA gene has a similar effect as in FIG. 9D (FIGS. 17 & 18), but less so. Therefore, the loss of SPA genes has an additive effect providing opportunities to determine the best combination of SPA genes to remove to achieve maximum photosynthetic capacity while minimising other possible negative effects on the plant. This ability to “mix and match” may be crucial in tailoring photosynthetic capacity to different growth conditions and agronomic practices.

### Discussion

### The Time Series HL Transcriptomics Data Indicates the Initiation of Dynamic Acclimation Processes

The exposure to a 7.5-fold increase in PPFD (HL; see Methods) presents both a threat and an opportunity to the plants in this study. The threat comes from the possibility that the PPFD will continue to increase and render the plant susceptible to irreversible photoinhibition. The opportunity comes from enhancing photosynthesis by initiating dynamic acclimation (FIG. 2A-D). Accompanying enhanced photosynthesis was also a lowering of reliance on the dissipation of excitation energy using NPQ, which can limit plant productivity (Kromdiijk et al., 2016).

The adaptation to a potential increase in photooxidative stress and photoinhibition is the early (≤1 h) strong but transient change in transcript abundance of 257 genes in clusters 21-26, upon exposure to HL. Clusters 22, 23, 25 and 26 include among them 64 known genes that promote abiotic stress tolerance (FIG. 1A, B). The transiently enhanced expression of these genes presumably allows the plant to overcome any potential initial detrimental effects of the HL exposure, as many other studies have reported (e.g. Ball et al., 2004; Gadjev et al., 2006; Ramel et al., 2012; 2013; Willems et al., 2016; Crisp et al., 2017; Huang et al., 2019).

Coordinated alteration in specific biological processes was evident in some clusters. Down-regulated clusters include those collectively associated with aspects of basal or innate resistance to pathogens (Underwood, 2012; Piasecka et al., 2015). Examples include genes coding for cell wall modifications and callose deposition (cluster 1), defense response to bacteria (cluster 3) and glucosinolate/glycosinolate biosynthesis (cluster 10). In this study, plants were grown at a PPFD below their light saturated rate of photosynthesis (Asat; FIG. 2C; see Methods). Plants grown under such light-limiting conditions may initially have to re-allocate resources away from some cellular processes in order to begin acclimation and take advantage of a sustained increase in PPFD. Photosynthetically active expanded but not senescing leaves, such as leaf 7 used here (see Methods), have been suggested to maintain a higher degree of poising of immunity to respond to biotic stress compared with abiotic stress (Berens et al., 2019). Therefore, in a converse situation where a potential abiotic stress threat emerges, the diversion of resources from defense against pathogens may be an appropriate response. Meanwhile, among the DEG time series clusters whose transcript levels increased at various points in the experiment, are those that could be preparing the leaf to increase its photosynthetic and metabolic capacity in order to begin acclimation (Eberhard et al 2008; Dietz, 2015). Genes in over-represented GO BP terms included those involved in macromolecule synthesis and especially translation (clusters 41-43) and related metabolic processes such as enhanced amino acid and organic acid biosynthesis (cluster 39).

A single exposure to 4 h HL is not sufficient to induce dynamic acclimation at the physiological level, requiring, under our conditions, a further 3 daily episodes of 4 h HL for this to begin to occur (FIG. 2A-D). This observation is consistent with a previous study where dynamic acclimation took around 5 days to be fully manifested and 2-3 days to discern any change in photosynthesis rates after a permanent shift from a photoperiod PPFD of 100 μmol m-2 s-1 to 400 μmol m-2 s-1 (Athanasiou et al., 2010). However, it should be noted that the HL regime used did not produce dynamic acclimation for the Col-0 accession but did for others such as Ws-2 and Ler-0 (Athanasiou et al., 2010). In contrast, the shorter more intense PPFDs used in this study induced dynamic acclimation in Col-0 and also Ws-0 (FIG. 2A-D; FIG. 4A-D; FIG. 8A-E).

### BBX32 Connects a Range of Cellular Processes to Dynamic Acclimation

Of all the comparisons carried out with relevant transcriptomics data sets, the most extensive overlap with time series HL DEGs was with those from dark-germinated phyA/phyB seedlings exposed to red light. While this was initially surprising because of the very different experimental conditions, earlier studies had shown a strong influence of photoreceptor genes (CRYs and PHYs) on photosynthetic capacity in Arabidopsis grown at a range of PPFDs (Walters et al., 1999) and an impact on the induction of some HL-responsive genes (Kleine et al., 2007; Shaikali et al., 2012; Guo et al., 2017). 91 light- and PHYA/B-regulated (co)TF genes were selected in this study. The HL time series expression data from these genes was subjected to VBSSM, which after two iterations, inferred a highly interconnected BBX32-centric (co)TF GRN (FIG. 3 and see Results). In the GRN, >50% of the nodes (genes) were subsequently confirmed by RNAseq to be influenced significantly in their expression by BBX32 (FIG. 3; FIG. 5). Over-expression of BBX32 clearly demonstrated that this gene is a negative regulator of dynamic acclimation (FIGS. 4A-D) and has an extensive influence on immediate responses to HL that include processes associated with photosynthesis (FIG. 6). Notably, depressed levels of LHCB4.3 transcript in BBX32-OE HL and LL plants (FIG. 6) could be important since levels of this antenna protein are closely linked to the degree of long-term acclimation to HL (Albanese et al., 2016).

BBX32 overexpression also impacts on a range of cellular processes that can be associated with basal immunity, including multiple GO designations for glucosinolate/glycosinolate metabolism, callose deposition, responses to chitin and to pathogens. This observation is consistent with the enrichment of the same processes noted in down-regulated temporal clusters (see above) and supports the suggestion that in wild type plants, down-regulation of basal immunity may be a necessary prerequisite for successful dynamic acclimation (see above). It is proposed that BBX32 control of aspects of basal immunity is part of its regulation of the initiation of dynamic acclimation.

BBX32 showed a greater transcript abundance over LL controls at any point onwards from the 2 h HL timepoint. Nevertheless, its transcript abundance was on a downward trend through the diel, paralleling its LL pattern of expression. Interestingly, while BBX32-OE plants displayed a 66-fold elevated BBX32 transcript level in LL, this value reduced to 33-fold after 3.5 h HL. The enhanced BBX32 expression in these plants is driven by the CaMV 35S promoter (Holtan et al., 2011), therefore the decline in transcript abundance over a diel could indicate that a temporal post-transcriptional control operates to determine BBX32 transcript levels.

The strong negative impact of BBX32 over-expression upon dynamic acclimation suggested that a defective gene ought to confer a converse elevated phenotype. The mutant bbx32-1 (see Results; Holtan et al., 2011), displayed a weakly significant trend of enhanced PSII operating efficiency compared with Col-0 between days 2 and 4 of the 5 days of 4 h HL exposure (FIG. 4B). This genotype, however, is unlikely to be a null mutant. The mutagenic T-DNA is inserted such that the first 172 amino acid residues of BBX32 would still be produced and a truncated transcript spanning this region has been detected in bbx32-1 seedlings (Holtan et al., 2011). The retained N-terminal region coded by this allele harbors the single B-Box zinc finger domain of BBX32 (Gangappa and Botto, 2014) and downstream sequences to residue 88, capable of binding at least one target protein, the transcription regulator EMBRYONIC FLOWER1 (EMF1; Park et al., 2011). The possibility of a partially functional truncated BBX32 may explain the weak phenotype of bbx32-1 with respect to this acclimation phenotype (FIG. 4B) and also its mild constitutive photomorphogenic phenotype in seedlings (Holtan et al., 2011).

### Two Levels of Control of Dynamic Acclimation

The time series data and the VBSSM led us to identify BBX32 and HY5 as strong negative and positive regulators respectively of dynamic acclimation in mature leaves (FIG. 4A-D; FIG. 8D, E) and places the start of the process right at the first hours of exposure to HL. This represents new functions for these two important genes and extends their role to cover a further dimension in the interaction of the mature plant with its light environment. In seedlings, HY5 controls the positive regulation of chlorophyll content, transcript levels of PhaGs in cool temperatures (Toledo-Ortiz et al., 2014) and the control of chloroplast development during photomorphogenesis (Ruckle et al., 2007), which suggests, along with data shown here (FIG. 6), that control of these photosynthesis associated processes by a BBX32/HY5-regulatory module is retained throughout the life of the plant. Furthermore, it is established that BBX32 fulfills the criterion of regulating both immediate responses to HL and the resulting dynamic acclimation, thus providing a link between these temporally distinct processes and experimental support for the hypothesis proposed here and by Eberhard et al. 2008.

The comparison drawn between the control of photomorphogenesis and dynamic acclimation was extended by establishing that CRY1, PHYB and one or more members of the PIF family are positive regulators of dynamic acclimation (FIG. 8A, C; FIG. 9E), while COP1 and one or more of SPA1, SPA2 and SPA3, are negative regulators (FIG. 9B, C). Again, by analogy with seedling photomorphogenesis, it is suggested that these genes act together to suppress dynamic acclimation under LL conditions by enabling the ubiquitin-mediated degradation of HY5 and other TFs. In HL, this suppression would be reversed by CRY1 physically interacting with and inhibiting the action of COP1/SPA protein (Laubinger et al., 2004; Lian et al., 2011, Lau and Deng 2012; Huang et al., 2014; Gangappa and Botto, 2016; Hoecker, 2017; Pham et al., 2018; Lau et al., 2019). Consequently, CRY1 would cause the re-direction of HY5 to the activation of dynamic acclimation. However, a further adaptation may be required to slow or accelerate dynamic acclimation. For example, to fine tune the establishment of dynamic acclimation in a fluctuating light environment. It is suggested that under HL, when HY5 is free of negative regulation by COP1/SPA, that BBX32 is the important additional moderator of the establishment of dynamic acclimation.

The scheme in FIG. 10 shows how this system may work. The transcriptional control of HY5 and by extension, other members of the BBX32-centric GRN (FIG. 3; FIG. 10), could be subject to regulation by additional intracellular signals in HL, such as those from chloroplasts and hormones, serving to coordinate a range of cellular processes for dynamic acclimation (Hardkte et al, 2000; Galvez-Valdivieso et al 2009; Estavillo et al., 2011; Ramel et al., 2012; 2013; Dietz, 2015; Gangappa and Botto, 2016; Guo et al., 2016; Exposito-Rodriguez et al., 2017).

The opposing regulation of dynamic acclimation by BBX32 and HY5 could mean that some form of genetic interaction between these genes drives its establishment in a manner similar to their negative and positive regulation respectively of photomorphogenesis (Datta et al., 2007; Holtan et al., 2011; Xu et al., 2014; Gangappa and Botto, 2016). However, BBX32 does not bind DNA and has been proposed to act as transcription co-factor in complexes with several TFs (Park et al., 2011; Holtan et al., 2011; Ganagappa and Botto, 2016; Tripathi et al., 2017). Of relevance here, in a tripartite complex with BBX21, BBX32 has been suggested to diminish the binding of HY5 to its target promoters (Datta et al., 2007; Holtan et al., 2011; Xu et al., 2014; Gangappa and Botto, 2016). Therefore, alongside transcriptional control of HY5 by BBX32, there may also be this post-translational control of HY5 action by BBX32 during dynamic acclimation.

The proposed need for both a CRY1/COP1/SPA- and a BBX32-mediated control of dynamic acclimation (FIG. 10) comes also from considerations about light quality and intensity. First, the fluence of blue light in the HL exposure used in this study would exceed the saturation of CRY1 activation, which is ca. 32-40 μmol m-2 s-1 blue light (Hoang et al., 2008; Liu et al., 2020). Therefore, while CRY1 signaling would need to be activated (i.e. on) for dynamic acclimation to happen, further signaling input may be required from other sources via BBX32 and its GRN to modulate the degree of response. A second factor is that at high fluence, CRY1 may produce H2O2 in the nucleus (Consentino et al., 2015). H2O2 for HL signaling is primarily synthesized and exported from chloroplasts and is dependent upon an active photosynthetic electron transport chain (Exposito-Rodriguez et al., 2017; Mullineaux et al., 2018). However, this does not exclude the possibility that the HL-dependent accumulation of H2O2 in nuclei may be augmented from other sources such as photo-saturated CRY1, signals from which could be fed into the BBX32-centric GRN.

In contrast to Arabidopsis grown at differing PPFDs but using similar fluorescent lighting to this study (Walters et al., 1999; see Methods), no influence of PHYA or PHYB was observed on dynamic acclimation. This could have been a consequence of the degree of blue light used in both the growth conditions and in applying a HL exposure (9% and 58% of total PPFD respectively; see Methods). This range of wavelengths in artificial lighting is typical of many controlled environment conditions (Naznin et al., 2019) and may have favored a response mediated by CRY1. The observation that plants harboring a constitutively active PHYB (YHB) allele displayed a partially accelerated acclimation phenotype (FIG. 8C) means that phytochromes could also control dynamic acclimation under some light environments and modify or interact with a CRY1-dependent signaling pathway (Ahmad et al., 2002; Yu et al., 2010). A further explanation could be that the PHY mutants were altered in leaf development such that this impacted on their photosynthetic properties. Equally, effects of a similar nature on BBX32-OE, hy5, cry1, cop1 and spa1, 2, 3 plants cannot be ruled out, but the clear lack of influence of a more severe dwarf shoot morphology on chloroplast level acclimation in det1-1 plants argues against this (FIG. 9A, D).

### BBX32 Over-Expressed in Arabidopsis and Soybean—Control of a Balance Between Photosynthetic Capacity and Leaf Longevity

Arabidopsis (At)BBX32 has been over-expressed in transgenic soybean (Glycine max. Merr., here called BBX32OE-soya) and year-on-year at different field sites produced up to an 8.5% increase in yield (Preuss et al., 2012). BBX32-OE Arabidopsis plants showed impaired dynamic acclimation and a consequent strong depression in Asat (FIG. 4C). This would appear to be at odds with the likelihood of an increased seed yield in the field. However, at the whole plant level, this effect may not be negative when considered as follows: BBX32-OE Arabidopsis plants show delayed flowering caused by the interaction of BBX32 with BBX4 (COL3), EMF1 and possibly other regulators of flowering time (Park et al., 2011; Tripathi et al., 2017). One consequence of delayed flowering is often the retardation of leaf senescence (Gan and Amasino, 1995; Wingler et al., 2010). Similarly, the BBX32OE-soya displayed an extended period of reproductive development associated with delayed leaf senescence thus contributing to the increased yield phenotype, similar to some “stay green” crop genotypes (Preuss et al., 2012; Kamal et al., 2019). While leaf senescence has not been measured in the BBX32-OE plants, nodes on the BBX32-centric GRN include GOLDEN-LIKE2 (GLK2) and ACTIVATING FACTOR1 (ATAF1; also called NO APICAL MERISTEM ATAF1/2 CUP SHAPED COTYLEDON (CUC)2 (NAC2); FIG. 3; FIG. 5). These genes are implicated in the maintenance of chloroplast integrity and are determinants of the entry of Arabidopsis leaves into senescence (Waters et al., 2009; Garapati et al., 2015; Song et al., 2018).

The potential negative effect of AtBBX32 over-expression in soybean depressing maximal photosynthetic capacity may not have proved detrimental because of the way the crop is grown commercially. Modern soybean varieties are grown as a row crop to achieve a high canopy coverage that maximises the absorption of light (Shepherd et al., 2018; Koester et al., 2014). Within the canopy, the photosynthetic rate of leaves below Asat may not have been significantly affected by AtBBX32 over expression in soya also as observed in Arabidopsis BBX32-OE plants (FIG. 4D). Furthermore, it is speculated that the photosynthetic capability of leaves exposed to full sun in BBX32OE-soya plants, while perhaps being unable to achieve a maximal Asat, benefitted from an enhanced leaf longevity and chloroplast integrity. A further effect could also have been that like their Arabidopsis counterparts, the BBX32OE soya plants had higher NPQ perhaps linked to enhanced PsbS transcript levels (FIG. 6), PsbS being a key determinant of the qE component of NPQ (Li et al., 2000). Consequently, the BBX32OE-soya plants may have been less susceptible to photooxidative stress. Thus, while raised NPQ could have lowered photosynthetic efficiency and diverted excitation energy away from photosynthesis, this may have had a protective effect under certain field and agronomic conditions and consequently improving overall crop performance.

In summary, that a network of TF genes could control dynamic acclimation, encompassing a wide range of cellular processes, implies a complex and extensive regulation that would provide resilience and flexibility in being able to accommodate input from further intracellular and extracellular signaling. At the whole plant level, this would allow for the degree of photosynthetic capacity and acclimation in individual leaves to be adjusted according to their specific micro-environments making this acclimation a truly dynamic process.

### Methods

### Growth Conditions

Plants were grown in an 8 h photoperiod (short day) at a PPFD of 150±10 μmol m⁻² s⁻¹ under fluorescent tubes (Phillips TLD 58W, 830 (warm whites), 22±1° C., 1 KPa vapour pressure deficit (VPD) and cultivation conditions as described previously (Bechtold et al., 2016; Windram et al., 2012). Unless stated otherwise, all plants were used from 35 to 40 days post-germination (dpg).

### Arabidopsis Genotypes

The following Arabidopsis mutants and transgenic lines, all in a Col-0 background, have been described previously: BBX32-10, BBX32-12, bbx32-1 (Holtan et al., 2011), hy5-215 (Oyama et al., 1997), hy5-2 (Ruckle et al., 2007), pifq (Leivar et al., 2008), cop1-4 (Deng and Quail, 1992), det1-1 (Chory et al., 1989), spa1/spa2/spa3 (spa1,2,3; Laubinger et al., 2004), phyA-219 (Reed et al., 1994), phyB-9 (Yoshida et al., 2018), cry1-304 (Ahmad and Cashmore, 1993), cry2-1 (Guo et al., 1998) and phyBY276H(YHB; Jones et al., 2015).

Identification of the cry1M32 Mutant

Based upon earlier research (Galvez-Valdivieso et al., 2009; Gorecka et al., 2014) in which a possible role for heterotrimeric G protein mediated high light (HL) signalling was studied, candidate genes coding for 7 transmembrane proteins that may have a role as G protein coupled receptors were sought to be identified. A collection of 59 T-DNA insertion mutants in genes coding for putative 7-transmembrane proteins (Moriyama et al., 2006) was screened for perturbed chlorophyll fluorescence in response to HL exposure (see below). The screening revealed that the insertion line Sail_1238_E12 (hereafter termed M32) was deficient in dynamic acclimation (FIG. 8B). The information available on T-DNA flanking sequences indicated that this was a T-DNA insertion in the first exon of At4g21570, a gene encoding a transmembrane protein of unknown function. However, complementation of M32 by transformation with the wild type At4g21570 gene did not restore a wild type phenotype (data not shown).

Besides being defective in dynamic acclimation, M32 was impaired in blue light inhibition of hypocotyl elongation under both low and high blue light fluence, accumulated less chlorophylls and anthocyanins than Col-0 under blue light, and presented delayed flowering time when grown in short day photoperiod. Later and in the light of the subsequent hypothesis that CRY1-mediated signaling controls dynamic acclimation in Arabidopsis (see Results and Discussion), it was realised that M32 resembled the phenotype of known cry1 mutants. Therefore, it was tested if CRY1 was altered in this mutant. CRY1 was amplified from its genomic DNA and the PCR product was Sanger sequenced on both strands. Col-0 CRY1 amplicon was also sequenced. The analysis of the sequence showed that in M32, CRY1 contains a single point mutation (GaA), which caused a substitution of Gly347Arg mutation in CRY1. This mutation was previously identified in a screening of EMS-mutagenized Arabidopsis seedlings (Ahmad et al., 1995) and designated as hy4-15, and affects the domain comprising the photolyase signature sequence. As a consequence, hy4-15 plants produce a wild type amount of full length CRY1, but the protein is not functional. Therefore, it was concluded that the M32 mutant is in fact a cry1 mutant that was named cry1M32.

### HL Exposures

The HL exposure was a PPFD of 1100 (±100) μmol m-2 s-1 from a white light emitting diode (LED) array (Isolight 4000; Technologica Ltd, Colchester UK) as described previously (Gorecka et al., 2014) and which permitted the simultaneous exposure of 9 plants. The HL exposure raised leaf temperature by 5° C. within 5 min of exposure which remained at this level for the remainder of the experiment (Gorecka et al., 2014). To determine the effect of this raised temperature (and the accompanying change in VPD) on the wider leaf transcriptome, a microarray analysis was performed on plants exposed to HL for 30 mins, or 27° C. under LL for 30 mins (LL/27° C.) compared with LL/22° C. control plants. There were 609 DEGs (1.5-fold change; FDR<0.05) that responded to HL and/or LL/27° C. Of these DEGs, 73 responded to the temperature increase alone. Given the small number, these were not eliminated from the time series data but none of the (co)TF DEGs fell into this group. For the HL time series transcriptomics, two consecutive sowings, 24 h apart, were grown to 35 dpg on the same growth room shelf and randomized across the shelf every day. Leaf 7 (Boyes et al., 2001) was tagged at 30 dpg. This staging of plant growth and 3 LED Iso light arrays were used to treat 27 plants each day. The HL exposure began 1 h after subjective dawn and was completed 1 h before subjective dusk. Each set of tagged leaves (4) at each HL time point and their LL controls (4) were sampled within 5 min at time 0.5 h and each 0.5 h interval for the 6 h exposure. Two HL experiments were conducted with duplicate samplings of 777 a full range of time points on each day. In addition, 4 time zero samples were processed for the 0 h time point. Both HL experiments provided a total of 100 samples for RNA extraction. These were 4 biological replicates (i.e. 4 sampled leaves) per timepoint per HL treatment (48 samples) and LL control (48 samples) plus 4 zero time point samples. To elicit dynamic acclimation, plants were subjected to 4 h HL, followed by a 0.5 h dark adaptation and then exposed to a range of actinic PPFDs (over 50 min) to collect CF data (see below). This HL treatment was repeated daily and CF data collected from the same plants for 5 consecutive days or on Days 1 and 5 only as stated.

### CF Measurements and Imaging

During the time series HL experiments, CF measurements were taken from leaf 7 of one plant in situ under each isolight using PAM-2000 portable modulated fluorimeters (PAM-2000, Walz GmbH, Effeltrich, Germany). At the end of each experiment the dark-adapted CF parameter Fv/Fm was determined for the same plants and LL controls and then again 24 h after being returned to growth conditions. For dynamic acclimation experiments, photosynthetic efficiency was estimated with a CF imaging system (Fluorimager, Technologica Ltd, Colchester, UK), exposing the plants to increasing actinic PPFD from 200-to-1400 μmol m⁻² s⁻¹ in 200 μmol m⁻² s⁻¹ steps every 5 min as described previously (Barbagallo et al., 2003; Gorecka et al., 2014). Whole rosette CF images were collected at each PPFD and processed using software (Technologica Ltd) to collect numerical data typically from fully expanded leaves (≥4 per plant) for Fq′/Fm′, Fv′/Fm′ and Fq′/Fv′ (Barbagallo et al., 2003; Baker, 2008, Gorecka et al., 2014). In some experiments, the diminished size of mutant plants rendered image processing problematic and in such stated cases, whole rosette data were collected. The raw data were fed via Excel into a program in R to calculate, plot and statistically analyse the CF parameters (Gorecka et al., 2014). The fluoroimager software produces average data of all leaf pixel values. CF parameters were represented as mean±SE from a minimum of 4 plants, and statistical significance was estimated with ANOVA followed by a post-hoc TukeyHSD test.

### Measurement of Photosynthesis

A was measured on leaf 7 of plants at 49 dpg using an 809 infrared gas exchange system (CIRAS-1, PP Systems, Amesbury, MA, USA). The response of A to changes in the intercellular CO2 concentration (Ci) was measured under a saturating PPFD, provided by a combination of red and white LEDs (PP Systems, Amesbury, MA, USA). In addition, the response of A to changes in PPFD from saturating to sub saturating levels was measured using the same light source at the current atmospheric CO₂ concentration (390 μmol mol-1). All gas analysis was made at a leaf temperature of 20 (±1) ° C. and a VPD of 1 (±0.2) KPa. Plants were sampled between 1 and 4 hours after the beginning of the photoperiod. For each leaf, steady state rates of A at current atmospheric [CO2] were recorded at the beginning of each measurement.

### Relative Ion Leakage

The method described by Overmyer et al. (2008) was followed. Briefly, leaves were collected from plants and placed in 5 ml de-ionized water, incubated with rotary shaking (100 rpm) for 4 h and the conductivity of the solution determined with a conductivity meter (Mettler Toledo, Leicester, UK) calibrated according to the manufacturer's instructions. Leaves were frozen overnight, thawed and conductivity measured again. Relative ion leakage was expressed as ‘conductivity after 4 h’/‘conductivity after freeze-thawing’.

### RNA Extraction, Labelling and Hybridisation to Microarrays

For the time series HL experiment, RNA was extracted from leaf 7 samples, labelled and hybridised to CATMA (a Complete Arabidopsis Transcriptome MicroArray) microarrays (v3; Sclep et al., 2007) as described by Breeze et al. (2011). Two technical replicates were used per biological replicate. Four biological replicates with a total of 13 time points per treatment (HL and LL) were analysed in this way, resulting in a highly replicated high-resolution time series of expression profiles. The hybridisation of labelled cDNA samples' experimental design for the HL and LL time series followed a statistically randomised loop design, which enabled expression to be determined at different time points both within and between treatments. After hybridization and washing, microarrays were scanned for Cy3 and Cy5 fluorescence and analysed as below. The raw and processed data are deposited with NCBI GEO (GSE78251).

### Analysis of Microarray Data

This has been described in detail previously (Breeze et al., 2011; Windram et al., 2012). Briefly, a mixed model analysis using MAANOVA (Wu et al., 2003; Breeze et al., 2011) was used with the same random (dye, array slides) and fixed variables (time point, treatments and biological replicate) to test the interaction between these factors as for the analysis of time series microarray data for senescing, Botrytis cinerea-infected, Pseudomonas syringae-infected and drought-stressed leaves (Breeze et al., 2011; Windram et al., 2012; Lewis et al., 2015; Bechtold et al., 2016).

Predicted means were calculated for each gene probe for each of the combinations of treatment, biological replicate and time point and for each of the combinations of treatment and time point from averages of the biological replicates. A GP2S Bayes' factor (Stegle et al., 2010) was used to rank probes and genes in order of likelihood of differential expression over the whole of the time series. Inspection of selected probes from the rank order of likelihood of differential expression was used to identify significant changes in expression with a Bayes' factor cut-off >10 giving 4069 probes corresponding to 3844 DEGs.

### Clustering of Gene Expression Profiles

The expression patterns of the identified DEGs in HL and LL were co-clustered with SplineCluster (Heard et al., 2005), using the mean expression profiles of the biological replicates generated from MAANOVA and a prior precision value of 0.001 as described previously (Windram et al., 2012; Bechtold et al., 2016).

### GO Analysis

GO annotation analysis was performed using DAVID (Huang et al., 2008) or AGRIGO (Du et al., 2010) with the GO Biological Process (BP) category (Ashburner et al., 2000). Overrepresented GO_BP categories were identified using a hypergeometric test with an FDR threshold of 0.05 compared against the whole annotated genome as the reference set.

Comparisons with Published Transcriptomics Data

The 3844 HL DEGs were compared on a cluster-by-cluster basis with publicly available transcriptomics data. Each DEG list from published data was mapped to AGI codes when necessary, cleaned to obtain single AGI codes since in some microarray data, probes mapped to several genes or were listed as “no_match” and were eliminated from the list. Overlaps within each cluster and their statistical significance were determined using a Hypergeometric Distribution Test (phyper function in R (v3.2.1)) in a custom R script, available upon request. When required, Venn diagrams of overlaps between Data Sets were plotted with Venny (http://bioinfogp.cnb.csic.es/tools/venny/index.html) and the significance of the overlaps calculated using the R phyper function.

### VBSSM

A full description of the VBSSM applied to this type of time series transcriptomics data is provided in Bechtold et al. (2016). The individual expression data for each biological replicate (n=4) for selected DEGs in HL was run through the VBSSM algorithm (Beal et al., 2005) on a local server at the University of Essex (Bechtold et al., 2016) to generate the GRNs as described in Results. The VBSSM output files were imported, mapped and plotted with Cytoscape (Shannon et al., 2003; http://www.cytoscape.org/).

### Expression Profiling by RNAseq

Mature (non-senescent) leaves were excised from 4 biological replicate plants per treatment, their total RNA extracted, and quality controlled as previously described (Albihlal et al., 2018). Library construction after mRNA enrichment and double stranded cDNA synthesis carried out using Illumina protocols by Novogene (UK) Ltd (Cambridge, UK; en.novogene.com/). Library sequencing was carried out on an Illumina HiSeq 4000 with a 150 base paired end reads to a depth of 20 million. Extraction and quality control of data from raw fastq files was carried out using the program CASAVA (Hosseini et al., 2010). The mapping of reads to the TAIR10 Arabidopsis genome sequence, followed by sorting and indexing of BAM output files was carried out using default settings in the program HISAT2 (v2.0.5; Kim et al., 2015). Across all samples, >92.5% of bases read attained the Q30 score threshold.

Transcript assembly and quantification was as fragments per kilobase of transcript sequence per million base pairs sequenced (FPKM) using HTseq (in union mode; Anders et al., 2015). Determination of differential expression between different genotypes and treatments was done using the program DEseq2 (Love et al., 2014) after read count normalisation and an adjusted p value threshold of <0.05 (negative binomial distribution p value model and FDR correction). Raw and processed data files were deposited in NCBI Gene Expression Omnibus (GSE158898).

### Locus Codes of Genes Mentioned Herein

Any one or more of the following gene sequences may be retrieved from publicly available databases (e.g. TAIR or the like) and are incorporated herein by reference:

AT1G01720, ATAF1; AT1G04400, CRY2; AT1G06180, MYB13; AT1G09100, AAA910 ATPase; AT1G09570, PHYA; AT1G14150, PnsL2; AT1G14920, GAI; AT1G16300, GAPCP-2; AT1G22190, RAP2.4; AT1G22640, MYB3; AT1G25540, PFT1; AT1G25550, MYB-like; AT1 G29910, Lhcb1/CAB3; AT1 G29920, CAB2/LHCII; AT1 G29930, CAB1/LHCII; AT1 G43670, FBPASE; AT1 G44575, PsbS; AT1 G49720, ABF1; AT1 G50420, SCL3; AT1G50640, ERF3; AT1G68520, BBX14; AT1G69010, B1M2; AT1G69490, NAP; AT1G70000, MYBD; AT1G75540, BBX21; AT1G76100, PETE1; AT1G76570, LHCB7; AT1 G77450, NAC032; AT1 G79550, PGK; AT2G01290, RPI2; AT2G05070, LHCB2; AT2G18790, PHYB; AT2G21330, FBA1; AT2G24540, AFR; AT2G24790, BBX4; AT2G27510, FD3; AT2G28350, ARF10; AT2G30790, PSBP-2; AT2G32950, COP1; AT2G34430, LHB1B1; AT2G34720, NFYA4; AT2G35940, BLH1; AT2G40100, LHCB4.3; AT2G40970, MYBC1; AT2G43010, PIF4; AT2G46270, GBF3; AT2G46340, SPA1; AT3G08940, LHCB4.2; AT3G09640, APX2; AT3G21150, BBX32; AT3G27690, LHCB2.3; AT3G60750, TK; AT3G61190, BAP1; AT4G05180, PSBQ-2; AT4G05390, RFNR1; AT4G08920, CRY1; AT4G10180, DET1; AT4G10340, LHCB5; AT4G15090, FAR1; AT4G17460, HAT1; AT4G29190, OZF2; AT4G32730, PC-MYB1; AT4G38960, BBX19; AT5G01600, FER1; AT5G07580, ERF106; AT5G08520, MYBS2; AT5G11260, HY5; AT5G11530, EMF1; AT5G12840, NF-YA1; AT5G15210, HB30; AT5G28450, LHC1; AT5G38420, RBCS2B; AT5G38430, RBCS1B; AT5G42520, BPC6; AT5G43270, SPL2; AT5G44190, GLK2; AT5G51190, ERF105; AT5G61270, PIF7; AT5G61590, ERF107; AT5G62000, ARF2; AT5G65310, HB5; AT5G67300, MYBR1; AT5G67420, LBD37; ATCG00020, PSBA; ATCG00270, PSBD; ATCG00300, PSBZ; ATCG00350, PSAA.

### REFERENCES

Adamiec, M., Drath, M. and Jackowski, G. (2008) Redox state of plastoquinone pool regulates expression of Arabidopsis thaliana genes in response to elevated irradiance. Acta Biochimica Polonica 55: 161-173.
Adams III, W. W., Cohu, C. M., Amiard, V. and Demming-Adams, B (2014) Associations between the acclimation of phloem-cell wall ingrowths in minor veins and maximal photosynthesis rate. Frontiers Plant Sci. 5: article 24.
Ahmad, M. and Cashmore, A. R. (1993) HY4 gene of A. thaliana encodes a protein with characteristics of a blue-light photoreceptor. Nature 366: 162-166.
Ahmad, M., Lin, C. and Cashmore, A. R. (1995) Mutations throughout an Arabidopsis blue-light photoreceptor impair blue-light-responsive anthocyanin accumulation and inhibition of hypocotyl elongation. Plant J. 8: 653-658.
Ahmad, M., Grancher, N., Heil, M., Black, R. M., Giovani, B., Galland, P. and Lardemer, D. (2002) Action spectrum for cryptochrome-dependent hypocotyl growth inhibition in Arabidopsis. Plant Physiol. 129: 774-785.
Albanese, P., Manfedi, M., Meneghesso, A., Marengo, E., Saracco, G., Barber, J., Morosinotto, T. and Pagliano, C. (2016) Dynamic reorganization of photosystem II supercomplexes in response to variations in light intensities. Biochim. Biophys. Acta 1857: 1651-1660.
Albihlal, W. S., Obomighie, I., Blein, T., Persad, R., Chernukhin, I., Crespi, M., Bechtold, U. and Mullineaux, P. M. (2018) Arabidopsis HEAT SHOCK TRANSCRIPTION FACTORA1b regulates multiple developmental genes under benign and stress conditions. J. Exp. Bot. 69: 2847-2862.
Allahverdiyeva, Y., Battchikova, N., Brosche, M., Fujii, H., Kangasjarvi, S., Mulo, P., Mahonen, A. P., Nieminen, K., Overmyer, K., Salojarvi, J. and Wrzaczek, M. (2015) Integration of photosynthesis, development and stress as an opportunity for plant biology. New Phytol. 208: 647-655.
Ashburner, M., Ball, C. A., Blake, J. A., Botstein, D., Butler, H., Cherry, J. M., Davis, A. P., Dolinski, K., Dwight, S. S., Eppig, J. T., Harris, M. A., Hill, D. P., Issel-Tarver, L., Kasarskis, A., Lewis, S., Matese, J. C., Richardson, J. E., Ringwald, M., Rubin, G. M. and Sherlock, G. (2000) Gene Ontology: tool for the unification of biology. Nat. Genet. 25: 25-29.
Athanasiou, K., Dyson, E. C., Webster, R. E. and Johnson, G. N. (2010) Dynamic acclimation of photosynthesis increases plant fitness in changing environments. Plant Physiol. 152: 366-373.
Badger, M. R., von Caemmerer, S., Ruuska, S. and Nakano, H. (2000) Electron flow to oxygen in higher plants and algae: rates and control of direct photoreduction (Mehler reaction) and rubisco oxygenase. Phil. Trans. R. Soc. Lond. B 355: 1433-1446.
Baker, N. R. (2008) Chlorophyll fluorescence: A probe of photosynthesis in vivo. Annu. Rev. Plant Biol. 59: 89-113.
Ball, L., Accotto, G. P., Bechtold, U., Creissen, G., Funck, D., Jimenez, A., Kular, B., Leyland, N., Mejia-Carranza, J., Reynolds, H., Karpinski, S. and Mullineaux, P. M. (2004) Evidence for a direct link between glutathione biosynthesis and stress defense gene expression in Arabidopsis. Plant Cell 16: 2448-2462.
Barbagallo, R. P., Oxborough, K., Pallett, K. E. and Baker, N. R. (2003) Rapid, non invasive screening for perturbations of metabolism and plant growth using chlorophyll fluorescence imaging. Plant Physiol. 132: 485-493.
Beal, M. J., Falciani, F., Ghahramani, Z., Rangel, C. and Wild, D. L. (2005) A Bayesian approach to reconstructing genetic regulatory networks with hidden factors. Bioinformatics 21: 349-356.
Bechtold, U., Richard, O., Zamboni, A., Gapper, C., Geisler, M., Pogson, B., Karpinski, S. and Mullineaux, P. M. (2008) Impact of chloroplastic- and extracellular 1054 sourced ROS on high light-responsive gene expression in Arabidopsis. J. Exp. Bot. 59: 121-133
Bechtold, U., Lawson, T., Meyer, R. C., Mejia-Carranza, J., Brown, I. R., Altmann, T., Ton, J. and Mullineaux, P. M. (2010) Constitutive salicylic acid defences do not compromise seed yield, drought tolerance and water productivity in the Arabidopsis accession C24. Plant Cell Environ. 33: 1959-1973.
Bechtold, U., Penfold, C. A., Jenkins, D. J., Legaie, R., Moore, J. D., Lawson, T., Matthews, J. S. A., Vialet-Chabrand, S. R. M., Baxter, L., Subramaniam, S., Hickman, R., Florance, H., Sambles, C., Salmon, D. L., Feil, R., Bowden, L., Hill, C., Baker, N. R., Lunn, J. E., Finkenstadt, B., Mead, A., Buchanan-Wollaston, V., Beynon, J., Rand, D. A., Wild, D. L., Denby, K. J., Ott, S., Smirnoff, N. and Mullineaux, P. M. (2016) Time-series transcriptomics reveals that AGAMOUS-LIKE22 affects primary metabolism and developmental processes in drought-stressed Arabidopsis. Plant Cell 28: in press doi/10.1105/tpc.15.00910.
Benjamini, Y. and Hochberg, Y. (1995) Controlling the false discovery rate: a practical and powerful approach to multiple testing. J.R. Statist. Soc. B. 57: 289-300.
Berens, M. L., Wolinska, K. W., Spaepen, S., Ziegler, J., Nobori, T., Nair, A., Kruler, V., Winkelmuller, T. M., Wang, Y., Mine, A., Becker, D., Garrido-Oter, R., Schulze-Lefert, P. and Tsuda, K. (2019) Balancing trade-offs between biotic and abiotic stress responses through leaf age-dependent variation in stress hormone cross-talk Proc. Natl. Acad. Sci. USA 116: 2364-2373.
Boyes, D. C., Zayed, A. M., Ascenzi, R., McCaskill, A. J., Hoffman, N. E., Davis, K. R. and Gorlach, J. (2001) Growth stage-based phenotypic analysis of Arabidopsis. A model for high throughput functional genomics in plants. Plant Cell 13: 1499-1510.
Breeze, E., Harrison, E., McHattie, S., Hughes, L., Hickman, R., Hill, C., Kiddle, S., Kim, Y-S., Penfold, C. A., Jenkins, D., Zhang, C., Morris, K., Jenner, C., Jackson, s., Thomas, B., Tabrett, A., Legaie, R., Moore, J. D., Wild, D. L., Ott, S., Rand, D., Beynon, J., Denby, K., Mead, A. and Buchanan-Wollaston, V. (2011) High-resolution temporal profiling of transcripts during Arabidopsis leaf senescence reveals a distinct chronology of processes and regulation. Plant Cell 23: 873-894.
Chory, J. and Peto, C. A. (1990) Mutations in the DET1 gene affect cell-type-specific expression of light-regulated genes and chloroplast development in Arabidopsis. Proc. Natl. Acad. Sci. USA 87: 8776-8780.
Chen, D., Xu, G., Tang, W., Jing, Y., Ji, Q., Fei, Z. and Lin, R. (2013) Antagonistic basic helix-loop-helix/bZIP transcription factors for transcriptional modules that integrate light and reactive oxygen species signalling in Arabidopsis. Plant Cell 25: 1657-1673.
Chory, J., Peto, C., Feinbaum, R., Pratt, L. and Ausubel, F. (1989) Arabidopsis thaliana mutant that develops as a light-grown plant in the absence of light. Cell 58: 991-999.
Consenting, L., Lambert, S., Martino, C., Jourdan, N., Bouchet, P.-E., Witczak, J., Castello, P., El-Esawi, M., Corbineau, F., d'Harlingue, A. and Ahmad, M. (2015) Blue-light dependent reactive oxygen species formation by Arabidopsis cryptochrome may define a novel evolutionary conserved signaling mechanism. New Phytol. 206: 1450-1462.
Crisp, P. A., Ganguly, D. R., Smith, A. B., Murray, K. D., Estavillo, G. Z., Searle, I., Ford, E., Bogdanovic, O., Lister, R., Borevitz, J. O., Eichten, S. R. and Pogson, B. J. (2017). Rapid recovery gene downregulation during excess light stress and recovery in Arabidopsis. Plant Cell 29: 1836-1863.
Datta, S., Hettiarachchi, C., Johansson, H. and Holm, M. (2007) SALT TOLERANCE HOMOLOG2, a B-Box protein in Arabidopsis that activates transcription and positively regulates light-mediated development. Plant Cell 19: 3242-3255.
Davletova, S., Rizhsky, L., Liang, H. J., Zhong, S. Q., Oliver, D. J., Coutu, J., Shulaev, V., Schlauch, K. and Mittler, R. (2005) Cytosolic ASCORBATE PEROXIDASE1 is a central component of the reactive oxygen gene network of Arabidopsis. Plant Cell 17: 268-281.
Deng, X.-W. and Quail, P. W. (1992) Genetic and phenotypic characterization of cop1 mutants of Arabidopsis thaliana. Plant J. 2: 83-95.
Diaz, M., De Haro, V., Munoz, R. and Quiles, M. J. (2007) Chlororespiration is involved in the adaptation of Brassica plants to heat and high light intensity. Plant Cell Environ. 30: 1578-1585.
Dietz, K. J., Horling, F., Konig, J. and Baier, M. (2002) The function of the chloroplast 2-cysteine peroxiredoxin in peroxide detoxification and its regulation. J. Exp. Bot. 53: 1321-1329.
Dietz, K.-J. (2015) Efficient high light acclimation involves rapid 1119 processes at multiple mechanistic levels. J. Exp. Bot. 66: 2401-2414.
Dong, J., Tang, D., Gao, Z., Yu, R., Li, K., He, H., Terzaghi, W., Deng, X. W. and Chen, H. (2014) Arabidopsis DE-ETIOLATED1 represses photomorphogenesis by positively regulating phytochrome-interacting factors in the dark. Plant Cell 26: 3630-3645.
Driever, S. and Baker, N. R. (2010) The water-water cycle in leaves is not a major alternative electron transport sink for dissipation of excess excitation energy when CO₂ assimilation is restricted. Plant Cell Environ. 34: 837-846.
Drozak, A. and Romanowska, E. (2006) Acclimation of mesophyll and bundle sheath chloroplasts of maize to different irradiances during growth. Biochim. Biophys. Acta 1757: 1539-1546.
Du, Z., Zhou, X., Ling, Y., Zhang, Z. and Su, Z. (2010) agriGO: a GO analysis toolkit for the agricultural community. Nucl. Acids Res. 38: W64-W70.
Eberhard, S., Finazzi, G. and Wollman, F.-A. (2008). The dynamics of photosynthesis. Annu. Rev. Genet. 42: 463-515.
Estavillo, G. M., Crisp, P. A., Pomsiriwong, W., Wirtz, M., Collinge, D., Carrie, C., Giraud, E., Whelan, J., David, P., Javot, H., Brearley, C., Hell, R., Marin, E. and Pogson, B. J. (2011) Evidence for a SAL1-PAP chloroplast retrograde pathway that functions in drought and high light signalling in Arabidopsis. Plant Cell 23: 3992-4012.
Exposito-Rodriguez, M., Laissue, P. P., Yvon-Durocher, G., Smirnoff, N. and Mullineaux, P. M. (2017). Photosynthesis-dependent H2O2 transfer from chloroplasts to nuclei provides a high-light signalling mechanism. Nature Comms. 8: art. 49.
Gadjev, I., Vanderauwera, S., Gechev, T. S., Laloi, C., Minkov, I. N., Shulaev, V., Apel, K., Inze, D., Mittler, R. and Van Breusegem, F. (2006) Transcriptomic footprints disclose specificity of reactive oxygen species signaling in Arabidopsis. Plant Physiol. 141: 436-455.
Galvez-Valdivieso, G., Fryer, M. J., Lawson, T., Slattery, K., Truman, W., Smirnoff, N., Asami, T., Davies, W. J., Jones, A. M., Baker, N. R. and Mullineaux, P. M. (2009) The high light response in Arabidopsis involves ABA signaling between vascular and bundle sheath cells. Plant Cell 21: 2143-2162.
Gan, S. and Amasino, R. M. (1995) Inhibition of leaf senescence by autoregulated production of cytokinin. Science 270: 1986-1988.
Gangappa, S. N. and Botto, J. F. (2014) The BBX family of plant transcription factors. Trends Plant Sci. 19: 460-470.
Gangappa, S. N. and Botto, J. F. (2016) The multifaceted roles of HY5 in plant growth and development. Mol. Plant 9: 1353-1365.
Gangappa, S. N. and Kumar, S. V. (2018) DET1 and COP1 modulate the coordination of growth and immunity in response to key seasonal signals in Arabidopsis. Cell Reports 25: 29-37.
Ganguly, D., Crisp, P., Harter, K., Pogson, B. J. and Albrecht-Borth, V. (2015) Genetic suppression of plant development and chloroplast biogenesis via the Snowy Cotyledon 3 and Phytochrome B pathways. Func. Plant Biol. 42: 676-686.
Garapati, P., Xue, G.-P., Munne-Bosch, S. and Balazadeh, S. Transcription factor ATAF1 in Arabidopsis promotes senescence by direct regulation of key chloroplast maintenance and senescence transcriptional cascades. (2015) Plant Physiol. 168: 1122-1139.
Gorecka, M., Alvarez-Fernandez, R., Slattery, K., McAusland, L., Davey, P. A., Karpinski, S., Lawson, T. and Mullineaux, P. M. (2014) Abscisic acid signalling determines susceptibility of bundle sheath cells to photoinhibition in high light exposed Arabidopsis leaves. Phil. Trans. R. Soc. B 369: 20130234.
Guo, H., Yang, H., Mockler, T. C. and Lin, C. Regulation of flowering time by Arabidopsis photoreceptors. Science 279: 1360-1363.
Guo, Z., Wang, F., Xiang, X., Ahammed, G. J., Wang, M., Onac, E., Zhou, J., Xia, X., Shi, K., Yin, X., Chen, K., Yu, J., Foyer, C. H. and Zhou, Y. (2016) Systemic induction of photosynthesis via illumination of the shoot apex is mediated sequentially by phytochrome B, auxin and hydrogen peroxide in tomato. Plant Physiol. 172: 1259-1272.
Hardtke, C. S., Gohda, K., Osterlund, M. T., Oyama, T., Okada, K. and Deng, X. W. (2000) HY5 stability and activity in Arabidopsis is regulated by phosphorylation in its COP1 binding domain. EMBO J. 19: 4997-5006.
Heard, N. A., Holmes, C. C., Stephens, D. A., Hand, D. J., and Dimopoulos, G. (2005). Bayesian co-clustering of Anopheles gene expression time series: Study of immune defense response to multiple experimental challenges. Proc. Natl. Acad. Sci. USA 102: 16939-16944.
Heyno, E., Innocenti, G., Lemaire, S. D., Issakadis-Bourguet, E. and Krieger-Liszkay, A. (2014) Putative role of the malate valve enzyme NADP-malate dehydrogenase in H2O2 signalling in Arabidopsis. Phil. Trans. R. Soc. B 369: 20130228.
Hoang, N., Schleicher, E., Kacprzak, S., Bouly, J. P., Picot, M., Wu, W., Berndt, A., Wolf, E., Bittl, R. and Ahmad, M (2008) Human and Drosophila cryptochromes are light activated by flavin photoreduction in living cells. PLoS Biol. 6: e160.
Hoecker, U. (2017) The activities of the E3 ubiquitin ligase COP1/SPA, a key repressor in light signalling. Curr. Op. Plant Biol. 37: 63-69.
Holtan, H. E., Bandong, S., Marion, C. M., Adam, L., Tiwari, S., Shen, Y., Mloof, J. N., Maszle, D. R., Ohto, M., Preuss, S., Meister, R., Petracek, M., Repetti, P. P., Reuber, L., Ratcliffe, O. J. and Khanna, R. (2011) BBX32, an Arabidopsis B-Box protein, functions in light signalling by suppressing HY5-regulated gene expression and interacting with STH2/BBX21. Plant Physiol. 156: 2109-2123.
Huang, D. W., Sherman, B. T. and Lempicki, R. A. (2008) Systematic and integrative analysis of large gene lists using DAVID bioinformatics resources. Nature Protocols 4: 44-57.
Huang, J., Zhao, X. and Chory J. (2019) The Arabidopsis transcriptome responds specifically and dynamically to high light stress. Cell Reports 29: 4186-4199.
Huang, X., Ouyang, X. and Deng, X. W. (2014) Beyond repression of photomorphogenesis: role switching of COP/DET/FUS in light signalling. Curr. Op. Plant Biol. 21: 96-103.
Inze, A., Vanderauwera, S., Hoeberichts, F. A., Vandorpe, M., Van Gaever, T. and Van Breusegem, F. (2012) A subcellular localization compendium of hydrogen peroxide-induced proteins. Plant Cell Environ. 35: 308-320.
Jones M. A., Hu, W., Litthauer, S., Lagarias, J. 1209 C. and Harmer, S. L. (2015) A constitutively active allele of phytochrome B maintains circadian robustness in the absence of light. Plant Physiol. 169: 814-825.
Jung, H.-S., Crisp, P. A., Estavillo, G. M., Cole, B., Hong, F., Mocklere, T. C., Pogson, B. J. and Chory, J. (2013) Subset of heat-shock transcription factors required for the early response of Arabidopsis to excess light. Proc. Natl. Acad. Sci. USA 110: 14474-14479.
Kamal, N. M., Gorafi, Y. S. A., Abdelrahman, M., Abdellatef, E. and Tsujimoto, H. (2019) Stay green trait: A prospective approach for yield potential, and drought and heat stress adaptation in globally important cereals. Int. J. Mol. Sci. 20: art. 5837.
Kangasjarvi, S., Neukermans, J., Li, S., Aro, E.-M. and Noctor, G. (2012) Photosynthesis, photorespiration, and light signalling in defence responses. J. Exp. Bot. 63: 1619-1636.
Karpinski, S., Escobar, C., Karpinska, B., Creissen, G. and Mullineaux, P. M. (1997) Photosynthetic electron transport regulates the expression of cytosolic ascorbate peroxidase gene in Arabidopsis during excess light stress. Plant Cell 9: 627-640.
Karpinski, S., Reynolds, H., Karpinska, B., Wingsle, G., Creissen, G. and Mullineaux, P. M. (1999) Systemic signalling and acclimation in response to excess excitation energy in Arabidopsis. Science 284: 654-657.
Kim, C., Meskauskiene, R., Zhang, S., Lee, K. P., Ashok, M. L., Blajecka, K., Herrfurth, C., Feussner, I. and Apel K. (2012) Chloroplasts of Arabidopsis are the source and a primary target of a plant-specific programmed cell death signaling pathway. Plant Cell 24: 3026-3039.
Kim, D., Langmead, B. and Salzberg, S. L. (2015) HISAT: a fast-spliced aligner with low memory requirements. Nature Methods 12: 357-360.
Kleine, T., Kindgren, P., Benedict, C., Hendrickson, L. and Strand A. (2007) Genome-wide gene expression analysis reveals a critical role for CRYPTOCHROME1 in the response of Arabidopsis to high irradiance. Plant Physiol. 144: 1391-1406.
Koester, R. P., Skoneczka, J. A., Cary, T. R., Diers, B. W. and Ainsworth, E. A. (2014) Historical gains in soybean (Glycine max Merr.) seed yield are driven by linear increases in light interception, energy conversion, and partitioning 1241 efficiencies. J. Exp. Bot. 65: 3311-3321.
Kromdiijk, J., Glowacka, K., Leonelli, L., Gabilly, S. T., Iwai, M., Niyogi, K. K. and Long, S. P. (2016) Improving photosynthesis and crop productivity by accelerating recovery from photoprotection. Science 354: 857-861.
Kumar, S. V. and Wigge, P. A. (2010) H2A.Z-containing nucleosomes mediate the thermosensory response in Arabidopsis. Cell 140: 136-147.
Lau, K., Podolec, R., Chappuis, R., Ulm, R. and Hothorn, M. (2019) Plant photoreceptors and their signalling components compete for binding to the ubiquitin ligase COP1 using their VP-peptide motifs. EMBO J. 38: e102140.
Lau, O. S. and Deng, X. W. (2012) The photomorphogenic repressors COP1 and DET1: 20 years later. Trends Plant Sci. 17: 584-593.
Laubinger, S., Fittinghoff, K. and Hoecker, U. (2004) The SPA quartet: A family of WD-repeat proteins with a central role in suppression of photomorphogenesis in Arabidopsis. Plant Cell 16: 2293-2306
Lawson, T., Davey, P. A., Yates, S. A., Bechtold, U., Baeshen, M., Baeshen, N., Mutwaktil, M. Z., Sabir, J., Baker, N. R., Mullineaux, P. M. (2014) C3 photosynthesis in the desert plant Rhazya stricta is fully functional at high temperatures and light intensities. New Phytol. 201: 862-873.
Lee, J., He, K., Stolc, V., Lee, H., Figueroa, P., Gao, Y., Tongprasit, W., Zhao, H., Lee, I. and Deng, X. W. (2007) Analysis of transcription factor HY5 genomic binding sites revealed its hierarchical role in light regulation of development. Plant Cell 19: 731-749.
Leivar, P., Monte, E., Oka, Y., Liu, T., Carle, C., Castillon, A., Huq, E. and Quail, P. H. Multiple phytochrome-interacting bHLH transcription factors repress premature seedling photomorphogenesis in darkness. Curr. Biol. 18: 1815-1823.
LePage, E., Zampini, E. and Brisson, N. (2013) Plastid genome instability leads to reactive oxygen species production and plastid-to-nucleus retrograde signalling in Arabidopsis. Plant Physiol. 163: 867-881.
Lewis, L. L., Polanski, K., de Torres-Zabala, M., Jayaraman, S. Bowden, L., Moore, J., Penfold, C. A., Jenkins, D. J., Hill, C., Baxter, L., Kulasekaran, S., Truman, W., Littlejohn G., Prusinska, J., Mead, A., Steinbrenner, J., Hickman, R., Rand, D., Wild, D. L., Ott, S., Buchanan-Wollaston, V., Smirnoff, N., Beynon, J., Denby, K. and Grant M. (2015) Transcriptional dynamics driving MAMP-triggered immunity and pathogen effector-mediated immunosuppression in Arabidopsis leaves following infection with Pseudomonas syringae pv tomato DC3000. Plant Cell 27: 3038-3064.
Li, K., Gao, Z., He, H., Terzaghi, W., Fan, L.-M., Deng, X. W. and Chen, H. (2015). Arabidopsis DET1 represses photomorphogenesis in part by negatively regulating DELLA protein abundance in darkness. Mol. Plant 8: 622-630.
Li, X., Bjorkman, O., Shih, C., Grossman, A. R., Rosenquist, M., Jansson, S. and Niyogi, K. K. (2000). A pigment-binding protein essential for regulation of photosynthetic light harvesting. Nature 403: 391-395.
Li, Z., Wakao, S., Fischer, B. B. and Niyogi, K. K. (2009) Sensing and responding to excess light. Ann. Rev. Plant Biol. 60: 239-260.
Lian, H.-L., He, S.-B., Zhang, Y.-C., Zhu, D.-M., Zhang, J. Y., Jia, K.-P., Sun, S.-X., Li, L. and Yang, H.-Q. (2011) Blue-light-dependent interaction of cryptochrome 1 with SPA1 defines a dynamic signaling mechanism. Genes Dev. 25:1023-1028.
Liu Q., Su T., He W., Ren H., Liu S., Chen Y., Gao L., Hu X., Lu H., Cao S., Huang Y., Wang X., Wang, Q., and Lin, C. (2020) Photooligomerization determines photosensitivity and photoreactivity of plant cryptochromes. Mol. Plant. 13, 398-413. Long, S. P., Humphries, S. and Falkowski, P. G. (1994) Photoinhibition of photosynthesis in nature. Ann. Rev. Plant Biol. 45: 633-662.
Love, M. I., Huber, W. and Anders, S. (2014. Moderated estimation of fold change and dispersion for RNA-seq data with DESeq2. Genome Biol., 15: 550.
Ma, D., Li, X., Guo, Y., Chu, J., Fang, S., Yan, C., Noel, J. P. and Liu, H. (2016) Cryptochrome 1 interacts with PIF4 to regulate high temperature-mediated hypocotyl elongation in response to blue light. Proc. Natl. Acad. Sci. USA 113: 224-229. Mittler, R., Vanderauwera, S., Gollery, M. and Van Breusegem, F. (2004) Reactive oxygen network of plants. Trends Plant Sci. 9: 490-498.
Moriyama, E. N., Strope, P. K., Opiyo, S. O., Chen, Z. and Jones, 1300 A. M. (2006). Mining the Arabidopsis thaliana genome for highly-divergent seven transmembrane receptors. Genome Biol. 7: R96.
Mullineaux, P. M., Exposito-Rodriguez, M., Laissue, P. P. and Smirnoff, N. (2018) ROS-dependent signaling pathways in plants and algae exposed to high light: Comparisons with other eukaryotes. Free Rad. Biol. Med. 122: 52-64.
Murchie, E. H. and Horton, P. (1997) Acclimation of photosynthesis to irradiance and spectral quality in British plant species: chlorophyll content, photosynthetic capacity and habitat preference. Plant Cell Environ. 20: 438-448.
Murchie, E. H., Hubbert, S., Peng, S. and Horton, P. (2005). Acclimation of photosynthesis to high irradiance in rice: gene expression and interactions with leaf development. J. Exp. Bot. 56: 449-460.
Naznin, M. T., Lefsrud, M., Gravel, V. and Azad, M. O. K. (2019) Blue light added with red LEDS enhance growth characteristics, pigments content, and antioxidant capacity in lettuce, spinach, kael, Basil and sweet pepper in a controlled environment. Plants 8: art. 93.
Oelze, M.-L., Vogel, M. O., Alsharafa, K., Kahmann, U., Viehhauser, A., Maurino, V. G. and Dietz, K.-J. (2012) Efficient acclimation of the chloroplast antioxidant defence of Arabidopsis thaliana leaves in response to a 10- or 100-fold light increment and the possible involvement of retrograde signals. J. Exp. Bot. 63: 1297-1313.
Oguchi, R., Hikosaki, K. and Hirose, T. (2003) Does the photosynthetic light 1322 acclimation need change in leaf anatomy? Plant Cell Environ. 26: 505-512.
Overmyer, K., Kollist, H., Tuominen, H., Betz, C., Langebartels, C., Wingsle, G., Kangasjarvi, S., Brader, G., Mullineaux, P. and Kangasjarvi, J. (2008) Complex phenotypic profiles leading to ozone sensitivity in Arabidopsis thaliana mutants. Plant Cell Environ. 31: 1237-1249.
Oyama, T., Shimura, Y. and Okada, K. (1997) The Arabidopsis HY5 gene encodes a bZIP protein that regulates stimulus-induced development of root and hypocotyl. Genes Dev. 11: 2983-2995.
Park, H.-Y., Lee, S.-Y., Seok, H.-Y., Kim, S.-H., Sung, Z.1330 R. and Moon, Y.-H. (2011) EMF1 interacts with EIP1, EIP6 or EIP9 involved in the regulation of flowering time in Arabidopsis. Plant Cell Physiol. 52: 1376-1388.
Pedmale, U., Huang, S. C., Zander, M., Cole, B. J., Hetzel, J., Ljung, K., Reis, P. A. B., Sridevi, P., Nito, K., Nery, J. R., Ecker, J. R. and Chory, J. (2015) Cryptochromes interact directly with PIFs to control plant growth in limiting blue light. Cell, 163: 233-245.
Penfold, C. A. and Wild, D. L (2011) How to infer gene networks from expression profiles, revisited. Interface Focus 1: 857-870.
Penfold, C. A. and Buchanan-Wollaston, V. (2014) Modelling transcriptional networks in leaf senescence. J. Exp. Bot. 65: 3859-3873.
Pham, V. C., Xu, X. and Huq, E. (2018) Molecular bases for the constitutive photomorphogenic phenotypes in Arabidopsis. Development 145: dev169870.
Piasecka, A., Jedrzejczak-Rey, N. and Bednarek, P. (2015) Secondary metabolites in plant innate immunity: conserved function of divergent chemicals. New Phytol. 206: 948-964.
Preuss, S. B., Meister, R., Xu, Q., Urwin, C. P., Tripodi, F. A., Screen, S. E., Anil, V. S., Zhu, S., Morrell, J. A., Liu, G., Ratcliffe, O. J., Reuber, L., Khanna, R., Goldman, B. S., Bell, E., Ziegler, T. E., McClerren, A. L., Ruff, T. G. and Petracek, M. E. (2012). Expression of the Arabidopsis thaliana BBX32 gene in soybean increases grain yield. PLoS ONE 7: e30717.
Ramel, F., Birtic, S., Ginies, C., Soubigopu-Taconnat, L., Triantaphylides, S. and Havaux, M. (2012) Carotenoid oxidation products are stress signals that mediate gene responses to singlet oxygen in plants. Proc. Natl. Acad. Sci. USA 109: 5535-1354 5540.
Ramel, F., Ksas, B., Akkari, E., Mialoundama, A. S., Monnet, F., Krieger-Liszkay, A., Ravanat, J.-L., Mueller, M. J., Bouvier, F. and Havaux, M. (2013) Light-induced acclimation of the Arabidopsis chlorina 1 mutant to singlet oxygen. Plant Cell, 25: 1445-1462.
Reed, J., Nagatani, A., Elich, T., Fagan, M. and Chory, J. (1994) Phytochrome A and phytochrome B have overlapping but distinct functions in Arabidopsis development. Plant Physiol. 104: 1139-1149.
Rossel, B., Wilson, P. B., Hussain, D., Woo, N. S., Gordon, M. J., Mewett, O. P., Howell, K. A., Whelan, J., Kazan, K. and Pogson, B. J. (2007) Systemic and intracellular responses to photooxidative stress in Arabidopsis. Plant Cell 19: 4091-4110.
Ruban, A. V. and Belgio, E. (2014) The relationship between maximum tolerated light intensity and photoprotective energy dissipation in the photosynthesis antenna: chloroplast gains and losses. Phil. Trans. R. Soc. B 369: 20130222.
Ruckle, M. E., DeMarco, S. M. and Larkin, R. M. (2007) Plastid signals remodel light signalling networks and are essential for efficient chloroplast biogenesis in Arabidopsis. Plant Cell 19: 3944-3960.
Schiebe, R. (2004) Malate valves to balance cellular energy supply. Physiol. Plant. 120: 21-26.
Schiebe, R. and Dietz, K.-J. (2012) Reduction-oxidation network for flexible adjustment of cellular metabolism in photoautotrophic cells. Plant Cell Environ. 35: 202-216.
Schottler, M. A. and Toth, S. Z. (2014) Photosynthetic complex stoichiometry dynamics in higher plants: environmental acclimation and photosynthetic flux control. Frontiers Plant Sci. 5: article 188.
Sclep, G., Allemeersch, J., Liechti, R., De Meyer, B., Beynon, J., Bhalerao, R., Moreau, Y., Nietfeld, W., Renou, J-P., Reymond, P., Kuiper, M. T. R. and Hilson, P. (2007) CATMA, a comprehensive genome-scale resource for silencing and transcript profiling of Arabidopsis genes. BMC Bioinformatics 8: 400.
Shaikhali, J., Barajas-Lopez, J. D., Otvos, K., Kremnev, D., Garcia, A. S., Srivastava, V., Wingsle, G., Bako, L. and Strand, A. (2012) The CRYPTOCHROME1-dependent response to excess light is mediated through the transcriptional activators ZINC FINGER PROTEIN EXPRESSED IN INFLORESCENCE MERISTEM LIKE1 and ZML2 in Arabidopsis. Plant Cell 24: 3009-3025.
Shannon, P., Markiel, A., Ozier, O., Baliga, N. S., Wang, J. T., Ramage, D., Amin, N., Schwikowski, B. and Ideker, T. (2003) Cytoscape: a software environment for integrated models of biomolecular interaction networks. Genome Res. 13: 2498-2504.

Shao, N., Duan, G. Y. and Bock, R. (2013) A mediator of singlet oxygen responses in Chlamydomonas reinhardtii and Arabidopsis identified by a luciferase-based genetic screen in algal cells. Plant Cell 25: 4209-4226.
Shepherd, M. J., Lindsey, L. E. and Lindsey, A. J. (2018). Soybean canopy cover measured with Canopeo compared with light interception. Agric. Environ. Lett. 3: 180031.
Shikata H., Hanada, K., Ushijima, T., Nakashima, M., Suzuki, Y. and Matsushita, T. (2014) Phytochrome controls alternative splicing to mediate light responses in Arabidopsis. Proc. Natl. Acad. Sci. USA 111: 18781-18786.
Song, Y., Jiang, Y., Kuai, B. and Li, L. (2018) CIRCADIAN CLOCK-ASSOCIATED 1 inhibits leaf senescence in Arabidopsis. Front. Plant Sci. 9: 280.
Stegle, O., Denby, K. J., Cooke, E. J., Wild, D. L., Ghahramani, Z., and Borgwardt, K. M. (2010). A robust Bayesian two-sample test for detecting intervals of differential gene expression in microarray time series. J. Comput. Biol. 17: 355-367.
Streb, P., Josse, E.-M., Gallouet, E., Baptist, F., Kuntz, M. and Cornic, G. (2005) Evidence for alternative electron sinks to photosynthetic carbon assimilation in the high mountain plant species Ranunculus glacialis. Plant Cell Environ. 28: 1123-1135.
Suorsa, M., Jarvi, S., Grieco, M., Nurmi, M., Pietrzykowska, M., Rantala, M., Kangasjarvi, S., Paakkarinen, V., Tikkanen, M., Jansson, S. and Aro, E.-M. (2012) PROTON GRADIENT REGULATIONS is essential for proper acclimation of Arabidopsis photosystem I to naturally and artificially fluctuating light conditions. Plant Cell. 24: 2934-2948.
Terashima, I., Hanba, Y. T., Tholen, D. and Niinemets, U. (2011) Leaf functional anatomy in relation to photosynthesis. Plant Physiol. 155: 108-116.
Toledo-Ortiz, G., Huq, E. and Quail, P. H. (2003) The Arabidopsis Basic/Helix-Loop-Helix transcription factor family. Plant Cell 15: 1749-1770.
Toledo-Ortiz, G., Johansson, H., Lee, K. P., Bou-Torrent, J., Stewart, K., Steel, G., Rodriguez-Concepcion, M. and Halliday, K. J. (2014) The HY5-PIF regulatory module coordinates light and temperature control of photosynthetic gene transcription. PLOS Genetics 10: e1004416.
Triantaphyllides, C., Krischke, M., Hoeberichts, F. A., Ksas, B., Gresser, G., Havaux, M., Van Breusegem, F. and Mueller, M. J. (2008) Singlet oxygen is the major reactive oxygen species involved in photooxidative damage to plants. Plant Physiol. 148: 960-968.
Tripathi, P., Carvallo, M., Hamilton, E. E., Preuss, S. and Kay, S. A. (2017)Arabidopsis B-BOX32 interacts with CONSTANS-LIKE3 to regulate flowering. Proc. Natl. Acad. Sci. USA 114: 172-177.
Underwood, W. (2012) The plant cell wall: a dynamic barrier against pathogen invasion. Frontiers Plant Sci. 3: Article 85.
Van Acken, O. and Whelan, J. (2012) Comparison of transcriptional changes to chloroplast and mitochondrial perturbations reveals common and specific responses in Arabidopsis. Fronters Plant Sci. 3: article 291.
Vandenabeele, S., Vanderauwera, S., Vuylsteke, M., Rombauts, S., Langebartels, C., Seidlitz, H. K., Zabeau, M., Van Montagu, M., Inze, D. and Van Breusegem, F. (2004) Catalase deficiency drastically affects gene expression induced by high light in Arabidopsis thaliana. Plant J. 39: 45-58.
van Rooijen, R., Aarts, M. G. M. and Harbinson, J. (2015) Natural genetic variation for acclimation of photosynthetic light use efficiency to growth irradiance in Arabidopsis. Plant Physiol. 167: 1412-1429.
Vermeirssen, V., De Clercq, I., Van Parys, T., Van Breusegem, F. and Van de Peer, Y. (2014) Arabidopsis ensemble reverse-engineered gene regulatory network discloses interconnected transcription factors in oxidative stress. Plant Cell 26: 4656-4679.
Vialet-Chabrand, S., Matthews J. S. A., Simkin, A. J., Raines, C. A and Lawson, T. (2017). Importance of fluctuations in light on plant photosynthetic acclimation. Plant Physiol. 173: 2163-2179.
Walters, R. G., Rogers, J. J. M., Shephard, F. and Horton, P. (1999) Acclimation of Arabidopsis thaliana to the light environment: the role of photoreceptors. Planta 209: 517-527.
Waters, M. T., Wang, P., Korkaric, M., Capper, R. G., Saunders, N. J. and Langdale, J. A. (2009) GLK transcription factors coordinate expression of the photosynthetic apparatus in Arabidopsis. Plant Cell 21: 1109-1128.
Willems, P., Mhamdi, A., Stael, S., Storme, V., Kerchev, P., Noctor, G., Gevaert, K. and Van Breusegem, F. (2016) The ROS wheel: Refining transcriptional footprints. Plant Physiol. 171: 1720-1733.
Windram, O., Madhou, P., McHattie, S., Hill, C., Hickman, R., Cooke, E., Jenkins, D. J., Penfold, C. A., Baxter, L., Breeze, E., Kiddie, S. J., Rhodes, J., Atwell, S., Kliebenstein, D. J., Kim, Y., Stegle, S., Borgwardt, K., Zhang, C., Tabrett, A., Legaie, R., Moore, J., Finkenstadt, B., Wild, D. L., Mead, A., Rand, D., Beynon, J., Ott, S., Buchanan-Wollaston, V. and Denby, K. J. (2012) Arabidopsis defense against Botrytis cinerea: Chronology and regulation deciphered by high-resolution temporal transcriptomic analysis. Plant Cell 24: 3530-3557.
Windram, O. and Denby, K. J. (2015) Modelling signalling networks underlying plant defence. Curr. Op. Plant Biol. 27: 165-171.
Wingler, A., Lea, P. J., Quick, W. P. and Leegood, R. C. (2000) Photorespiration: metabolic pathways and their role in stress protection. Phil. Trans. R. Soc. Lond. B 355: 1517-1529.
Wingler, A., Purdy, S. J., Edwards, S.-A., Chardon, F. and Masclaux-Daubresse, C. (2010) QTL analysis for sugar-regulated leaf senescence supports flowering dependent and -independent senescence pathways. New Phytol. 185: 420-433.
Wituszyńska, W., Ślesak, I., Vanderauwera, S., Szechyńska-Hebda, M., Kornaś, A., Van Der Kelen, K., Muhlenbock, P., Karpińska, B., Maćkowski, S., Van Breusegem, F. and Karpiński, Stanislaw (2013) LESION SIMULATING DISEASE1, ENHANCED DISEASE SUSCEPTIBILITY1, and PHYTOALEXIN DEFICIENT4 conditionally regulate cellular signaling homeostasis, photosynthesis, water use efficiency, and seed yield in Arabidopsis. Plant Physiol. 161: 1795-1805.
Xu, D., Li, J., Gangappa, S. N., Hettiarachchi, C., Lin, F., Andersson, M. X., Jiang, Y., Deng, X. W. and Holm, M. (2014) Convergence of light and ABA signalling on the ABI5 promoter. PLOS Genetics 10: e1004197.
Yoshida, Y., Sarmiento-Manus, R., Yamori, W., Ponce, M. R., Micol, J. L. and Tsukaya, H. (2018) The Arabidopsis phyB-9 mutant has a second-site mutation in the VENOSA4 gene that alters chloroplast size, photosynthetic traits, and leaf growth. Plant Physiol. 178: 3-6.
Yu, X., Liu, H., Klejnot, J. and Lin, C. (2010) The cryptochrome blue light receptors. The Arabidopsis Book e0135; doi: 10.1199/tab.0135.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of increasing photosynthetic capacity in a plant, the method comprising modulating Cryptochrome 1 (CRY1)-directed signalling in the plant.

2. The method of claim 1, wherein modulating CRY1-directed signalling comprises reducing or abolishing the expression of at least one nucleic acid sequence encoding a Suppressor of PhyA-105 (SPA) polypeptide and/or reducing or abolishing the activity of a SPA polypeptide in the plant, wherein optionally comprising reducing or abolishing the expression and/or activity of one, two or three SPA polypeptides, wherein the nucleic acid sequences are selected from SPA1, SPA2, SPA3 and/or SPA4.

3. (canceled)

4. The method of claim 1, wherein modulating CRY1-directed signalling comprises:
(a) reducing or abolishing the expression of at least one nucleic acid sequence encoding a B-Box Domain containing protein 32 (BBX32) polypeptide and/or reducing or abolishing the activity of a BBX32 polypeptide in the plant; and/or
(b) reducing or abolishing the expression of at least one nucleic acid sequence encoding a Constitutively Photomorphogenic (COP) polypeptide and/or reducing or abolishing the activity of a COP polypeptide in the plant; and/or
(c) increasing the expression of at least one nucleic acid sequence encoding a Cryptochrome 1 (CRY1), Long Hypocotyl 5 (HY5), Phytochrome Interacting Factor (PIF) and/or Phytochrome B (PHYB) polypeptide; and/or
(d) increasing the activity of a CRY, HY5, PIF and/or PHYB polypeptide in the plant.

5. (canceled)

6. (canceled)

7. The method of claim 1, wherein shoot growth of the plant is significantly the same or improved as compared to a control or wild-type plant, wherein optionally the PSII operating efficiency (Fq′/Fm′) of the plant is increased by about 40% or more as compared to a control or wild-type plant, wherein the actinic photosynthetically active photon flux density (PFFD) is about 800 μmol m⁻² s⁻¹.

8. (canceled)

9. The method of claim 1, wherein the method comprises introducing at least one mutation into the nucleic acid sequence encoding 5 the SPA, BBX32 and/or COP polypeptide(s), wherein optionally the mutation is a loss of function mutation, further optionally an insertion, deletion or substitution.

10. (canceled)

11. The method of claim 9, wherein:
(a) the mutation is introduced by insertional mutagenesis, optionally transposon mutagenesis;
(b) the mutation is identified by a Targeted Induced Local Lesions in Genomics (TILLING) method; or
(c) the mutation is introduced by genome-editing, optionally CRISPR/Cas9.

12. The method of claim 1, wherein a transgenic construct is introduced into the plant, wherein:
(a) the transgenic construct is capable of reducing or abolishing the expression of the nucleic acid encoding a SPA, BBX32 and/or COP polypeptide;
(b) the transgenic construct is capable of reducing or abolishing the activity of a SPA, BBX32 and/or COP polypeptide;
(c) the transgenic construct is capable of increasing the expression of the CRY1, HY5, PIF and/or PHYB polypeptide; or
(d) the transgenic construct is capable of increasing the activity of the CRY1, HY5, PIF and/or PHYB polypeptide.

13. The method of claim 1, wherein the plant is a crop plant, wherein optionally the crop plant is grown under controlled light conditions, wherein further optionally the light conditions are optimised depending on the photosynthetic capacity of the plant.

14. (canceled)

15. (canceled)

16. A plant, plant part or seed obtainable by the method of claim 1.

17. A plant having modulated CRY-directed signalling, wherein the PSII operating efficiency (Fq′/Fm′) in the plant is increased by about 40% or more as compared to a control or wild-type plant, wherein the PFFD is about 800 μmol m-2 s-1.

18. The plant of claim 17, wherein the plant has reduced or abolished expression of at least one nucleic acid encoding a SPA, BBX32 and/or COP polypeptide and/or reduced or abolished activity of a SPA, BBX32 and/or COP polypeptide as compared to a control or wildtype plant, wherein optionally the plant has increased expression of at least one nucleic acid sequence encoding a CRY, HY5, PIF and/or PHYB polypeptide and/or increased activity of a CRY, HY5, PIF and/or PHYB polypeptide as compared to a control or wild-type plant.

19. (canceled)

20. The plant of claim 17, wherein the plant is a crop plant, wherein optionally the plant comprises one or more mutations in at least one nucleic acid sequence encoding a SPA, BBX32 and/or COP polypeptide, wherein further optionally:
(a) the mutation is introduced by insertional mutagenesis, optionally transposon mutagenesis;
(b) the mutation is identified by a TILLING method; or
(c) the mutation is introduced by genome-editing, optionally CRISPR/Cas9.

21. (canceled)

22. (canceled)

23. The plant of claim 17, wherein the plant comprises a transgenic construct, wherein:
(a) the transgenic construct is capable of reducing or abolishing the expression of the nucleic acid encoding a SPA, BBX32 and/or COP polypeptide;
(b) the transgenic construct is capable of reducing or abolishing the activity of the SPA, BBX32 and/or COP polypeptide;
(c) the transgenic construct is capable of increasing the expression of the CRY1, HY5, PIF and/or PHYB polypeptide; or
(d) the transgenic construct is capable of increasing the activity of the CRY, HY5, PIF and/or PHYB polypeptide.

24. A method of identifying one or more alleles associated with increased photosynthetic capacity in one or more plants, the method comprising:
(a) detecting in the plant(s) one or more polymorphism(s) in a nucleic acid sequence encoding a SPA, BBX32, COP, CRY, HY5, PIF and/or PHYB polypeptide, wherein the one or more polymorphism(s) are 5 associated with increased photosynthetic capacity;
(b) identifying one or more allele(s) at the one or more polymorphism(s) that are associated with increased photosynthetic capacity.

25. The method of claim 24, further comprising introgressing the nucleic acid sequence comprising the one or more polymorphism(s) into a plant having increased photosynthetic capacity, wherein optionally the plant is a crop plant.

26. (canceled)

27. A method of producing a food or feed product in a plant grown under controlled light conditions, the method comprising:
(a) obtaining a plant having increased photosynthetic capacity according to the method of claim 1;
(b) isolating a plant part or seed from the plant; and
(c) producing a food or feed product from the plant part or seed.

28. A method of quantifying photosynthetic capacity in one or more plants having modulated CRY-directed signalling, wherein the method comprises:
(a) subjecting the plant to high light (HL) conditions;
(b) subjecting the plant to low light (LL) conditions;
(c) subjecting the plants to increasing actinic PPFD; and
(d) quantifying PSII operating efficiency, linear electron flux and/or quantum yield of CO₂ assimilation in the one or more plants.

29. The method of claim 28, wherein the plant has reduced or abolished expression of at least one nucleic acid sequence encoding a SPA, BBX32 and/or COP polypeptide and/or reduced or abolished activity of a SPA, BBX32 and/or COP polypeptide as compared to a control or wild-type plant, wherein optionally the plant has increased expression of at least one nucleic acid sequence encoding a CRY, HY5, PIF and/or PHYB polypeptide, and/or increased activity of a CRY, HY5, PIF and/or PHYB polypeptide as compared to a control or wild-type plant.

30. (canceled)

31. The method of claim 28, wherein:
(a) the HL conditions comprise a PFFD of about 1100 μmol m-2 s-2 optionally for about 4 hours;
hours;
(b) the LL conditions comprise a PFFD of about 100 μmol m-2 s-2 optionally for about 0.5 hours;
(c) the increasing actinic PPFD comprises between about 200 μmol m-2 s-2 to about 1400 μmol m-2 s-2 optionally in about 200 μmol m-2 s-2 steps for about 5 minutes per step; and/or
(d) chlorophyll fluorescence (CF) measurements are obtained to quantify PSII operating efficiency, linear electron flux and/or quantum yield of CO₂ assimilation in the one or more plants, optionally between 1 to 5 days, wherein optionally if the PRI operating efficiency (Fq′/Fm′) is about 40% or more wherein the actinic PFFD is about 800 μmol m-2 s-1, the plant is determined to have increased photosynthetic capacity as compared to a control or wild type plant.

32. (canceled)

33. Use of a plant according to claim 16 for producing a food or feed product, wherein optionally the food or feed product is a non-propagation material such as flour or oil.

34. (canceled)


SEQUENCE LISTING (22 sequences)

SEQ ID NO: 1 - DNA, 5375 bp, Arabidopsis thaliana
gaaaaaattaaataaaataatgttcaaaaaattgtccgccagaataaagaaaatttgatatgtggcttcaaagcctaaattaatcattaaaataaaaaatcagaaaataaggaatgaagaacgaattgtgcaaaagtctatagagaaagagagattcatttgtgtttcttcgactatacacagaatacaaaagagatattatatgtaattatcaatagtatatgacaaatttatagcctcacctttcttcttcttcttcttcttcttctgtgtgtttttgaatttgctttcttcaagaggtttcttctccacaccgcttcggtgtatgatctgattgtgatctctctcgatatctgggatcttcggggtaagcgatttgatttttgtgtatttctcgtttctcgtaatcttcatgattggttttaatagtttgctgcgattttacataatcttcttctgttttgattagctactttagattacagctaatgatacgtttcgggattagtgattattgtatgtttaactagttttagaatctctacatgcatgaatttactaggtagggcttctttcttcttcttcttcttcttcttcttcattttgttggtttgattctgatttgtttttgtattgtatatgatttaaggtatggaggctgtagattattattattattacgtgtggtgctgatgcaatgagtgttgatgcctgttatggaaagagtagctgaagaaactgtggcgactaacaacatacaattgaaagcacgagtggatgatgttccttgtaataagcttgatgctcgtcataacgatatggtgattcaatcagaaactgcaaacagtgattgtcctggaagctcagctcacaggaatgttgatttgactaagccgccgcctcctgaggaagccgcaggagctaagctttctgttgaagagttgacattggggaactacaggattgttcaagggagtaataatactaatgtggatagtcctagggctggcaagtttgagcatctttatcgtttggctagagggtctgcttttagggcaggagatggggatcttgattctcaacctcgtgatatggatcaaatgttgtccagaatcagacaacagcttgctggagctccttctgagaggcaaaacttgaaaccttttatgtctaggaggagcgatcagaatcttgaagctttttctgaacgtctaagagcagctggtgagaatagtattatgaatgctcctgcattgatcagtgaaggtgtccaaatgaaaacgccggtgagttcttcgaatttttctcagttgttacttaaaagagctatgaaagggaaaggggtagtaggaaagaaccaggaaactcctcctgaatttgtctctgatcaagatttaggaagcaaggaaaaaaagttggatattagcaagtcgcctactcctcacgatgtgttacctctaaaatcaagtcccaaaggaaatggtatggtatcacatggtgatgggaatcatagtaaatcttctattggaatcagtttgagggagtttcttagatcaagttatgctaaaagagaaaaaagacatggcctttgtctatttaggcagctggtggaactggttgactcagcccattcaaaaagattattcttgctagacttgagaccatcccttttcactttagttccatcaaagaaacttagatatattggtaattttggaaagaacgatctagaatcagatgttgacgaagatttgaataggaggaggcctgttgttgaggaatcatctagtggaggtagggattcgaagaaaagaaaaatggacttgcatttgaattctccagggaatcaacttcaggccacttcaaccgggcgacctttcaaaaggaaaagccctgtgattgatttaaacatggttgatgcacgtaatccagacagttgcgagcttcaacagcaagactacataaaaaatttaagcgtgtcttcagtgtcgagaaaacagtctatgtccacatggctagaagagcaatggtatacttgtccagaggagataaatggagaagatattggagaaaaatcaaatatttatgcccttggtgttcttctgtttgaggtatggacgaatacccagttttattgttgtggatagatttttagtttgctttaataccaagaaggatgtaacatttccttgcatttgaacactatgtactgtcatatcctgcttatgtgggctttgggtagtttatattctttcattttcatgaaatttcttactctagtctgaactttgaatcccagagctagtattttccagtctgcagtatataacttgtcagtatttttgtttacttgtttgatactttcatgtgaagacgcctactgacatctagcttgaccaattgttgtttcagttgctatgccattgcgagtcaggcgagatgcatgctgcaatgatggcagatttacgccatcggattctcccaccaacatttctctcgaaatacccgaaggaagctggattttgtctttggcttctacatcctgaaccctcctcccgaccatcagctaggtatttagctgagtgctactcttttatgattcatgtgggcgagtatttactaggagtctaggagtggtacaaaggagaatctgtagaccttccattagcttaaatagtcgtatttaagatttctttgccggatatcttcaattttatttaatgttttcccgcttaattgtgaagaaaaaaaatggcatgctactgtgtgaattatcttcttgtttctttttctgttctcattcttttctctttatgtgacagagatatactaaagtcagagttgatatgcgaggatgattcagttaaatcgactgctgctgctgaggagatatctgagctgttacttcattttttgtcttcattagaagtgcagaagaagaaaaaagcatctaaacttttgcaagatatccagaccttagaggatgatattaaggaggccgagcgaagatattcttcaaatgtatctctggtgagatctcatggagctattgaaaaaagggtgcaatcatctcccctagacgagcactgtacaacttccagtgccttgtttgtaccaactgctaatacagacaggctgatgagtaatatccgtcaacttgaagatgcatattttttcatgcgatcacaaatcaacttatcgagttctgctgctactgcccgttctgacaaaacactaaaggacagggacagatgctctgaaaaccaaaatgaaaatcaggatatgagtaccaaaggaaaatcttcagatcaactcgaagtgtttttcgaggggttgtgcaaatttgctcggtatagcaagttcgaaacctgtgggacaataagaagtggggaccttttaaactctgcaagtgtggtctgctcgttgagttttgaccctgatgaagaacacatagcagcagctgggatatcaaagaagatcaagatttttgacttcaatgcatttatgaatgaatctgtcggtgttcattatccactagtagagatggtcaacaaatctaaattaagctgtgtttgctggaatagctacatcaaaaactacttggcctcgactgactacgatggtgtagttcaggtacctatcgatgtcaagtttggaatctttcttatttaccatcatatgttgcacggtaggatctataggactagcctcatgtgctttctcttcctctatgagatgtgtacatgttaatcttgtgataagatagagatatagaagcgtctatttcatggttttcacttgccattttcttactgtatggtgctaatgaatgtagatatgggatgccggcactggacaaggattttcgcagtacacagaacaccagaagagagcctggtcagttgatttttctccatctgacccgacaaaatttgttagtggaagtgatgactgctcggtgaagctttggagcatcaatgaggcacttctctttcctttacctcctgatattgcaagaattttcatgtccccgttgtttttattactccagattatagctccataaggcatgattctctgtttatacttgtgaatttccaaatctagtgaaacttatattcttctctgtttggtatgcagaaacgttcattaggcacaatctggagtccagcaaatgtgtgttgcgtgcaattctcttcttattccaatcacttattggcatttgggtcagctgattacaaggtctattgctatgaccttcggtatgtcaaaacaccttggtgcacattggctggtcatgagaaagcggtgagttatgttaaatttatggattcagaaaccattgtttctgcttccactgataactctctcaagctttggaatcttaacaagacaaattcctctggtttatctcctggtgcttgttcattgacatataaaggacatacaaatcagaaggttagtcccaaaaacttttctatctacctttaacatctcatccaacatcattagttagttctgatgtctaatcctttacctgtgcagaatttcgttggattgtcggttttggacggatacatagcttgtggttcagagaccaatgaggtatatatcacatcataaaacaaaatctcaacctttagaatacttaatttcagattatcaaaggtggtatatgtcaatcgcgtttagtttgtttatgaagctgcctaaaattagttgttataacttactaaaaaaactttactttgattttgcgtcttaggtatatagttactataaatctttaccgatgccaatgacttcatacaagtttggatccgtcgatcccatttctggaaacgaatactttgatgacaatggacagtttgtgtcgagcgtctgttggaggaaaaaatcgaatatgcttgtagctgcaaactctactggaaacatgaagctactaaaacttgtttgacaacttggccttttcgctactactgcgggcattggactttttgagtctgaaactaggacaaagcaggtctgactttgaagcaaacgtttttgtcctggaaactatgatcccatatgaccagaagagaaaaaaagttcgaaatcttggagaagttgaagaagcaaaactgtctgtgataatatattatttgattcttcttcctgtaattctatatatatgagatataatttgagataaacttctcagattcttcagaaatgattcatatttggtcggcataacgtgtaatgatgagaggtatataacttgtacaagtgtgttatatcgttagctcggttatgtaattgttgttggagattctctataagagagaaaatatatatatcatccattcatttcatcgcatggctactacctc

SEQ ID NO: 2 - protein, 1029 aa, Arabidopsis thaliana
MPVMERVAEETVATNNIQLKARVDDVPCNKLDARHNDMVIQSETANSDCPGSSAHRNVDLTKPPPPEEAAGAKLSVEELTLGNYRIVQGSNNTNVDSPRAGKFEHLYRLARGSAFRAGDGDLDSQPRDMDQMLSRIRQQLAGAPSERQNLKPFMSRRSDQNLEAFSERLRAAGENSIMNAPALISEGVQMKTPVSSSNFSQLLLKRAMKGKGVVGKNQETPPEFVSDQDLGSKEKKLDISKSPTPHDVLPLKSSPKGNGMVSHGDGNHSKSSIGISLREFLRSSYAKREKRHGLCLFRQLVELVDSAHSKRLFLLDLRPSLFTLVPSKKLRYIGNFGKNDLESDVDEDLNRRRPVVEESSSGGRDSKKRKMDLHLNSPGNQLQATSTGRPFKRKSPVIDLNMVDARNPDSCELQQQDYIKNLSVSSVSRKQSMSTWLEEQWYTCPEEINGEDIGEKSNIYALGVLLFELLCHCESGEMHAAMMADLRHRILPPTFLSKYPKEAGFCLWLLHPEPSSRPSARDILKSELICEDDSVKSTAAAEEISELLLHFLSSLEVQKKKKASKLLQDIQTLEDDIKEAERRYSSNVSLVRSHGAIEKRVQSSPLDEHCTTSSALFVPTANTDRLMSNIRQLEDAYFFMRSQINLSSSAATARSDKTLKDRDRCSENQNENQDMSTKGKSSDQLEVFFEGLCKFARYSKFETCGTIRSGDLLNSASVVCSLSFDPDEEHIAAAGISKKIKIFDFNAFMNESVGVHYPLVEMVNKSKLSCVCWNSYIKNYLASTDYDGVVQIWDAGTGQGFSQYTEHQKRAWSVDFSPSDPTKFVSGSDDCSVKLWSINEKRSLGTIWSPANVCCVQFSSYSNHLLAFGSADYKVYCYDLRYVKTPWCTLAGHEKAVSYVKFMDSETIVSASTDNSLKLWNLNKTNSSGLSPGACSLTYKGHTNQKNFVGLSVLDGYIACGSETNEVYSYYKSLPMPMTSYKFGSVDPISGNEYFDDNGQFVSSVCWRKKSNMLVAANSTGNMKLLKLV

SEQ ID NO: 3 - DNA, 5621 bp, Arabidopsis thaliana
cttacttaaacacagtcaaattcattttctgccttagaaaagatttttatcgaaaatcgacgtttttgaaaaaactcaaattatcgtcgttttgttctcagatttcttctgctctcttcttcttctccttcttcttcgttccaccgcctctgttgctttatcttcttcttccttccttcgattgttgattacgtcggtggatctttgttctcctctgtgttgttttcattgctagatttcgtcaatgattggcttctcacgattcgatttttccggctcctgttcttaatttcctctgaggtagattagtaaaactctttgtctcgtaaacattgttcgatgatgaaattagaaagttctcactcttgtttgccaaatttgggatatttctggaattgaaaatgaatcgtcatttttttcaatttctgaggattttgttacaattgttttttatttatgttagaattgagattgtgtgatgaatatcagtaaccctagacaattgtgaaatcaaagctatgaaaatcttttgatgctatgtttcctatgatacagagaatgatggatgagggatcagtaggggatgtgagtaggattgatgaagctgatgttgctcaccttcaattcaagaatagtgagcagtcgtttaaaccggagaacattgaagtccgggaagtaaaagaagtgcaggttcagagagaagctggttctcctgattgttcttatggtgtaatagcagattttttagatggtaagaatggtggtgaccatgtggagcttatagggaatgaaccatgttctagccggcagaatacaaatgatgagggggatgttgtcgaggagttgactgttaaaacttgtgagggatcgagtatggctatagttggtagaccaagtagtagagctagattagagatgaatcgcagtcagtttctgcatcgttttccgctagacggtgatttaccgggtagtagttctatgagtaagaaggtaattgacaggggaacagtaagtatcttgagaaatgctgggaaaatgtctttgcctgagacttcaaatggacaactggctattattgctgttaatggagaagctaatgaacatttgacaaatgttgaacgaaatcctgtacctgtagaggcattgtctcatgaaggcatcaagacaaagatgttatcgcagtctgggttttctcagttttttgttagaaaaacattgaaagggaagggagttactttcagaggcccgccaaacaatagatctaaagcgaggaatatggatcagcaaactgtagctagttctggtagcgctttagtaatagctaatacttcagcaaagatttctagcagcatccctttggcggcttatgatggtttgccgtgcttaccttccaataccagtaaaccatcttcttgtgctaatccttcagatacacatagaggttgtggaggtgaaggtttaagtctgagagaatggttgaagtcggagcggcaggaagttaataaagcagaatgtatgtatatctttaggcagatcgtggatcatgtggactgttctcattctcaaggagttgttttatgtgacctgcgaccatcttcattcaaaattttcaaggaaaatgcagtaaagtatgtggtttcaggttctcaacgagagtcttttgacagcaacatgaacaaagagactttaagtcaactcgaaaaccctctggttagaagacggttaggagatacttctagtctctctatccctgcaaagaagcagaaatcaagtggaccaagctctagacagtggcctatgtttcaacgagctggtggcgtcaatatccaaactgaaaataatgatggcgcgatacaggagtttcatttcagatctagtcaacctcattgtagtacagttgcctgcccttttacttcagtgagtgagcagttggaagagaaatggtatgcgagccctgaggagctcaggggtgacatgcgttcagcctcttcaaacatctatagtttgggtattcttctttatgaggtatgccatcttaattgtagcttttgacctgcaattgtaagtatgtacatccatgagttgaaatgtgaatctgcaagcccttaatgttgacacacatatatatgttttcccgtgggattatgtctttaactgggacatatcgtgtgtgggtcatgtttgggtccagatagatttagatagtgtagcatgtttgttaatgagatggtaacagtgaagtttctcgttgaaagtacagttggctgcatggctcataggtttatgtatatattgattcgactgtttcttggtcaaaacataacattttcattgatatactaatttgtttttttccgtcagttgctgagccaatttcagtgtgaaagagctcgtgaggctgcaatgtctgatatccgacaccgcattcttcctcccaaatttctgtcggagaatccaaaggaagctgggttttgtctttggttacttcatccagaatcttcatgccgaccatcaaccaggcaatgcaaaatctttagctactataattctcaaatgatataaagcgtgttattgaacttttgtatgcttgtttctgttgatagtttaaggccatgaatccctagtgggtctattggaagataatcttgttgtatatctagaaatgtacatatgcaccaaatgtggaagttttgttctcctggctgtaaacgggaggacgattgtctcgtcaacagtttcacataggattgttaggttctactctgaaaaataattagtctttgtactaactgagctccctttatgtgttgatagtctccttcctgtcataatttatctgtcaagaatgtgagttggcttaatctctaaataagtttgatgttgttctgtattagggacatcttacaatctgaagtagttaatggaatcccggatttgtatgctgaaggcttatctttatccattgaacaagaggacacagagtctgaactcttgcagcatttccttttcttgtcccaagagaaaaggcagaagcatgctggaaatttgatggaagaaattgcgtccgtagaagcagatatcgaggaaattgtcaagagacgttgtgccatagggcctccgtccctggaagaagcttctagctcatctcctgcttctagtgtacctgaaatgagactgatcaggaacattaatcagcttgaaagcgcttatttcgctgcaaggatagacgcacatctccctgaagcacgttataggttacgcccagatagagatctgctaagaaatagtgataatactgtagcagaagtagaaaacagtgagacttggagctcagatgatcgtgttggagcgttctttgatgggttatgcaaatatgctcgatatagtaagtttgaaactcggggtgtgctgaggaccagtgagttgaacaacacttcaaatgtgatttgttctctgggttttgatcgggacgaggattattttgccacagctggtgtgtcgaaaaaaattaagatatatgagttcaattcccttttcaacgaatctgttgacattcattatccagccatagaaatgccaaatagatcaaagcttagcggcgtttgctggaacaactacattaggaactacctagcttcctctgattatgatggtattgtcaaggtatttgaacctttgaagttttacgtagattgtttatcacttgattacccctttatttccttcggtctaatgaatacaaacgtatttatgatgaatccagttatgggatgtgacaacaggacaggccatttcacatttcatagagcatgagaagagggcttggtctgttgacttctctgaagcttgtccgacaaaattagccagtggaagcgatgattgttctgtgaagctatggaacatcaatgaggcatgctcttctatactggattttgtcgttttaatagctactcctactgagatatctggcatatatacatatgaatatatcaatagcactttggattattctgtcaatgttgtcatgctcgtattttcagccttgtagctaatagacaatcttcttgtttgactgtgcagaggaactgcttaggaacaatcaggaacattgcaaacgtttgttgtgtgcaattttctccacagtcctctcacttgctagcctttggatcatcagacttcagaacctactgctatgatctaaggaatttaagaactccttggtgcatactgtcaggacacaacaaagcagttagctatgcgaagttcttggataatgaaactcttgtaacagcatcaactgataacacgttaaagctttgggatcttaagaaaaccactcacggtggcctctcaacaaatgcttgcagcttaacttttggcggccatacaaatgagaaggtagctaatgctgaaacgtaattttgagttaatctatataatgatgtttctctttttccttcgtggttgatttgtcttttcctgcctttttcagaactttgttggtttgtctacttctgatggatatatagcttgtggctctgagacaaatgaggtatgcccctacaaacctgggaactctttgatattcctataactttgtgtatggaatctccttttctataatcctttaacaagcattgccacagattgatagtttgattctcaacttaactagaagctccagagtttatttcatgtgatggaaaaaatcacagttccatgtgaattccgtctatttatacgcgatggaggaacttatccattgatactaacaattagtaaaataactggcgtagggtcaaaaaagaactaatctggtagtattgttgtttaagtgatctctctgattgagaaatatgctgtttcttgttttgttcaggtttatgcctatcaccggtctcttcccatgccaattacctcgtacaagtttggttctattgatccaatatcaggtaaggagatagaggaggacaacaacctgttcgtttcaagcgtttgctggagaaaaagatcgaatatggttgtttcagctagctcaaatgggtcaatcaaagttctacagttggtctgaaataaatacatacatacagaagaagctgaggtaattctacacctagagccctcttggtttatggtaaaacaaaaaccccatcaatccaagaatcaggcaaaacaacggagataacgaacaagctttcaacacacagatatgagatagttttatctatctgttgctaaagttttcctcaaatggggtgaaggaatgagagtgagtagaactatagttgttcaatagttgcggtttgatcagcgcggtaagggtttataccgctgatgggtatacatagtaaggtaaaagaccttctcttttgtggtgttctaaggtgtttatttttttattttttattttggctcttacaataagattcctctttgtgatatgtaaatttgctccgatagttctaaggtaaaagagcatactctgatgtatatatagttgaaatgagctttttagttctacggatgaatctcctccaccaagaaaagaaaaatcttcgtttttctttttaaaaactttaaatgcatcgttttttgttgaatgttcacaaaagtaatctttg

SEQ ID NO: 4 - protein, 1036 aa, Arabidopsis thaliana
MMDEGSVGDVSRIDEADVAHLQFKNSEQSFKPENIEVREVKEVQVQREAGSPDCSYGVIADFLDGKNGGDHVELIGNEPCSSRQNTNDEGDVVEELTVKTCEGSSMAIVGRPSSRARLEMNRSQFLHRFPLDGDLPGSSSMSKKVIDRGTVSILRNAGKMSLPETSNGQLAIIAVNGEANEHLTNVERNPVPVEALSHEGIKTKMLSQSGFSQFFVRKTLKGKGVTFRGPPNNRSKARNMDQQTVASSGSALVIANTSAKISSSIPLAAYDGLPCLPSNTSKPSSCANPSDTHRGCGGEGLSLREWLKSERQEVNKAECMYIFRQIVDHVDCSHSQGVVLCDLRPSSFKIFKENAVKYVVSGSQRESFDSNMNKETLSQLENPLVRRRLGDTSSLSIPAKKQKSSGPSSRQWPMFQRAGGVNIQTENNDGAIQEFHFRSSQPHCSTVACPFTSVSEQLEEKWYASPEELRGDMRSASSNIYSLGILLYELLSQFQCERAREAAMSDIRHRILPPKFLSENPKEAGFCLWLLHPESSCRPSTRDILQSEVVNGIPDLYAEGLSLSIEQEDTESELLQHFLFLSQEKRQKHAGNLMEEIASVEADIEEIVKRRCAIGPPSLEEASSSSPASSVPEMRLIRNINQLESAYFAARIDAHLPEARYRLRPDRDLLRNSDNTVAEVENSETWSSDDRVGAFFDGLCKYARYSKFETRGVLRTSELNNTSNVICSLGFDRDEDYFATAGVSKKIKIYEFNSLFNESVDIHYPAIEMPNRSKLSGVCWNNYIRNYLASSDYDGIVKLWDVTTGQAISHFIEHEKRAWSVDFSEACPTKLASGSDDCSVKLWNINERNCLGTIRNIANVCCVQFSPQSSHLLAFGSSDFRTYCYDLRNLRTPWCILSGHNKAVSYAKFLDNETLVTASTDNTLKLWDLKKTTHGGLSTNACSLTFGGHTNEKNFVGLSTSDGYIACGSETNEVYAYHRSLPMPITSYKFGSIDPISGKEIEEDNNLFVSSVCWRKRSNMVVSASSNGSIKVLQLV

SEQ ID NO: 5 - DNA, 3943 bp, Arabidopsis thaliana
cgacagtgaatcgtttctttcagcagtcgtttttttgtttttattctaaaaggccgagaatttatgaggctgagagagagagccttcttctgcttttttttccgagtttaaaactgtgtttggtgtttgtttgttgtgtggctctgtttcaatctcgtaaagattatttctgggtggcctctctcctttagatacttcttatcttctccctctcactaattttgcggtaaagaagcttcctttcttttttttggctctttacatgatcaaaagatttgcattttttcttgtatatatttttatcttttgagtgttgttgttcttaaatgggtctcctaggttcttgagttttcttctgtttatagcctagggtttgtttcttgagtgggatcacttaaaaaaggggctttacattgtgagagtctaagttcttggcgaatgtgtaacgatctttaagcaatgagctgttgaatccgaaatgattccagttagagttgggaatacagagaagtcattggttacgtgttggtttctggttagcttttggtggatgttaaatggaaggttcttcaaattctaactctaggggattcaatacctctggtgtttcagataggaacactgagttcttacctgttgagagactcactactcgttccaagccttcctctcatgttgatgagtatgtcagaagcttgtttggtagtactagcacacacaagtctggtgaagatgattctttagggattgatccttttgttagatccttggaatggggtgatgttagtttgagacagtggctggataaacctgaacgatctgttgatgttttcgagtgcttgcacgttttcagacaaattgttgagattgtgaatgcggctcactctcaaggcattgttgttcataatgtcaggccatcatgttttgtcatgtcttcgtttaaccatgtttcgttcattgaatctgcttcttgttcggactctggctctgattccttggaggatggtccaattagccagaaggaaatcggtagctcgagaagagaggaagctgtatcgaaagctattgcaatagaagagaaaggagtctacaataagttgttggagagaaaaatcgagaaactagaggaagaaaaaacccaaccttttccgatgaaacatatattagcaatggagacaagttggtacacaagccctgaagaggattttggttcttcaagtacttgtgcttcagatgtttaccgccttggcgttcttcttttcgaggtttgtgataaagaaagaaacattgtttctaacaaattagtaactgcttttggattcaaagtttctgtttttgttttgcgtgtttattttcttgaaaaggtgatgagatgaaagttagtccctttgcagctattttgccctgtaccttcaagagaagagaagtctagaactatgtctagtcttagacatcgcgttcttccgcctcagatattgctcaaatgtccaaaagaagcttctttctgcttgtggttactgcatcctgagccaacttgtcgaccatcaatgaggtttacatattgtcttatacttcatgttcttaggttgagcattgttagtctctgtcatcttttactacatggaactaatggggaatttgtcttttatggtgcagtgacttgctgcaaagcgagtttataactgaacctagagataatttggaagaacgtgaagcagcaatcgagcttcgggacagaatcgaggaacaggagtcgttactcgagttcttgttactgattcaacagaggaagcaagaatctgcgtatagattgcaggatactgtttcacttctttcttctgacattgagcaagttgtaaagagacagttaattttgaagaaaaggggaagctcactctctgatttcagtaaagatgatcatcaatatacctctggtcagccattgatgagtttccaggcaaacgaggagccatcagcattcttggcctcaagaaaacgggttagacaagggattctggcattggaaaacggtgttgaagttgatgaagagagtcaaggaagcacacttcttgaaagctcccggttgatgagaaacttcaagaaactagaatcggtctactttctgacaaggcgcaggcaaatgaaggctgctgcttcagggaaatcattaactagacattcgccgttaagtagtgaaaatggaagaggttctatgattgtctcagagaaaagttcagtaagcaacccggtggcacccaaagctttcttcaataatgactcgagacaaggggggtggatagatcctttccttgagggtttgtgcagatatttatcattcagtcagctaagagtgaaagctgatttgaaacaaggagatttgttgaactcttctaacctcgtttgcgctcttgcgtttgaccgagaaggagaattattcgccactgcgggagtcaacaagaaaatcaagatattcgaatgtaactccatagtaaacgataaccgggatattcactatccggtggtggaattggctggtcggtcaaagctaagtagtttatgttggaacagctacataaagagccaaattgcatcaagtaactttgatggtgttgttcaggttagttttacattcagctctagcgttattaaaaacttccagtttcataaaaccaatttgggatacatatgtttttgttttcagatatgggatgttgcaagaagccagttggttacagagatgaaggagcacaagaagcgagtatggtccatcgatatttcatcagcagacccgactttgctggctagcggaagcgatgatggaaccgttaagctctggagtatcaatcaggcaattctaatttgaaatgtcggctattgaaaactgaaactgatttctacaataatgcaaacatcttcattctcagaatgtatgattaagagtttgtttttcttggaaatgttttcagggagttagcattgggaccatcaagacaaaggccaatgtatgctgtgtccagtttccatcagactccggacggtctctagcatttggttctgcagatcataaagtgtattactacgatcttcgaaaccccaagattcctctgtgcacaatgattggtcatagcaagacagtgagttatgtcaagtttgtagattcatccactcttgtgtcctcttctactgataacacactgaagctttgggacttgtcgatgtctgcttctgggattaatgaatcccctcttcactcattcactggacacactaatttaaaggtaacattttaccctgaaatatgctttcaagactctcttcggtctccggtttctgagatgtaatgttcttgtgatgtgttgcagaactttgttggcttatcggtctctgatgggtatatagcaacaggctcagagactaacgaggtaaaggcttgatacagaaagaaaaatgaactttctgacagattgtctcctaaaaacttgattatctgagcttcttgttgttgtgaatctattacaggttttcgtgtaccacaaggcattcccaatgccagtgatgtcgtacatgttcaacaacactgactctatgtctggtctcgaagtggacgatgcatcacagttcatatcttcaatctgctggcgaggacagtcgtctacgttagtcgctgctaactccaatggcaacataaaaattctggagatgatgacttgagcctctcatagatctcttgttcttcttctccgcaagagatatgctctgctggagtgattgatatagggtaaaccatttttaccaaaaaaaaaaaaagataggaaagtggaaacattgtgtatagaatttctattgagaaaaaaaaaagaagagaggatacaaatattggatctttgtatttgtatattctgttataactgaaaacatctcaataaaaaaaggttataatttt

SEQ ID NO: 6 - protein, 837 aa, Arabidopsis thaliana
MEGSSNSNSRGFNTSGVSDRNTEFLPVERLTTRSKPSSHVDEYVRSLFGSTSTHKSGEDDSLGIDPFVRSLEWGDVSLRQWLDKPERSVDVFECLHVFRQIVEIVNAAHSQGIVVHNVRPSCFVMSSFNHVSFIESASCSDSGSDSLEDGPISQKEIGSSRREEAVSKAIAIEEKGVYNKLLERKIEKLEEEKTQPFPMKHILAMETSWYTSPEEDFGSSSTCASDVYRLGVLLFELFCPVPSREEKSRTMSSLRHRVLPPQILLKCPKEASFCLWLLHPEPTCRPSMSDLLQSEFITEPRDNLEEREAAIELRDRIEEQESLLEFLLLIQQRKQESAYRLQDTVSLLSSDIEQVVKRQLILKKRGSSLSDFSKDDHQYTSGQPLMSFQANEEPSAFLASRKRVRQGILALENGVEVDEESQGSTLLESSRLMRNFKKLESVYFLTRRRQMKAAASGKSLTRHSPLSSENGRGSMIVSEKSSVSNPVAPKAFFNNDSRQGGWIDPFLEGLCRYLSFSQLRVKADLKQGDLLNSSNLVCALAFDREGELFATAGVNKKIKIFECNSIVNDNRDIHYPVVELAGRSKLSSLCWNSYIKSQIASSNFDGVVQIWDVARSQLVTEMKEHKKRVWSIDISSADPTLLASGSDDGTGVSIGTIKTKANVCCVQFPSDSGRSLAFGSADHKVYYYDLRNPKIPLCTMIGHSKTVSYVKFVDSSTLVSSSTDNTLKLWDLSMSASGINESPLHSFTGHTNLKNFVGLSVSDGYIATGSETNEVFVYHKAFPMPVMSYMFNNTDSMSGLEVDDASQFISSICWRGQSSTLVAANSNGNIKILEMMT

SEQ ID NO: 7 - DNA, 3718 bp, Arabidopsis thaliana
ccaaaaccaaaatacgaaaaaggttttagctttacgttcttttgattcctaggtcgtcgtttaaccacgttgcttgcgtggaaagagaagagaagtgaagattatcaaaaagcgaaaaataaaaccctcactgtgaagtaaaaatcgtaggcccaaaatagatttctcttagccgagaatttaatttttatgaatctgattattgcttttctacgaactcagtttttgcgatgtgtggttctcaaaaaaggatcgtttcttcggtgcctcttagtctctcaagtacttcttcttcttcttatcttcttcttcgcttttcttttatatatagagagagtttagtggtcaagaagcttcctcgtgtttgtctctttatgtaatcaaaggttgcatttttattcgaatatattttctggttttggtgttttctgtcaaagggtcttttaaaaatcttaactttcgttctaaatttctttactttatttcgattctaacttagggcttttgggctgggatcatgaaatgaagtattgcagaatccagaggagtttgtttggtaatcaatgaagggttcttcagaatctagttccagaggattaaacaacacctctggtgtttcagagttttgtacagatggttctaagtctttgtctcacattgattatgtcagaagcttacttggttcacataaagaagctaacttgggaggacttgatgatgactctattgtcagagctttggaatgtgaggatgttagcttgcgtcagtggttagacaatccagaccgatccgttgatgcctttgagtgttttcatgttttcagacagattgttgagattgttaatgcagctcactctcaaggcattgttgttcataatgttaggccttcttgtttcgttatgtcttcgtttaacaatgtttcgtttatcgaatccgcttcttgttcggattccgggtctgatgaggatgcaacaacaaagtctcgagagattggtagctcgagacaagaggagattctatcagagagacgaagcaaacaacaagaggaagtaaagaagcaacctttccctatgaaacagatattagcaatggagatgagttggtatacaagtcatgaagaagataatggttctctgtgtaattgtgcttctgatatttaccgtcttggtgttcttctctttgaggtttgtgcgtaagaagatttgatttttgttcttaactgctttgttttcttgagaaaactgatttgatgaatgttagtctttgtgcagctattctgccctgtctcctcaagagaagagaagtcaagaactatgtctagtttaagacatcgtgttcttccccctcagatattactcaattggcctaaagaagcttccttttgcttgtggttactgcatcctgagcctagctgtcgaccatcgatgaggtgatgattgattctctctttgatgtttttagtttcaattggaaaagttttgtcctttaattaagagtattgtctcctttgtctttacttttgcagtgagttgctacaaagtgagtttatcaacgaacctagagagaatttggaagaacgtgaagctgcaatggagctacgggatagaatcgaggaacaagagttattactcgagttcttgtttctgattcaacagaggaaacaagaagctgcagacaaattgcaggatacaatatcgcttctctcttctgatatcgatcaagtcgtaaagcgacagttggttttacagcaaaaagggagagatgtccgttcgttcttagcctcaagaaaacggattagacaaggagctgagaccactgcagcagaggaagaaaatgatgataatagcattgatgaggaatctaagcttgatgatactctagaaagtacacttcttgaaagctctagattaatgagaaatttgaagaaactggaatcagtctactttgcaacacgatacagacaaatcaaggctgctactgctgctgaaaaaccgttggctagatattattcggcattgagttgtaacggtagaagttcagaaaagagttcaatgagccaaccatcaaaagatcctatcaatgattctagacaaggcgggtggatcgatccattcctcgagggtttgtgcaagtatttgtcttttagtaagctaagagtgaaagcagatttgaagcaaggagacttgttgaactcgtctaaccttgtttgcgcaattgggtttgatcgtgatggagagtttttcgccacggcaggcgtcaacaaaaagatcaagatattcgaatgcgagtcgataataaaagatggccgggacattcattaccctgttgtggaacttgctagccggtctaagcttagtggtatatgttggaacagttacattaagagccaagtcgcatcaagtaactttgaaggcgtggttcaagtaagttcagaagaaaactctcaaccttattaatcaatgaaaagtatgaaaaaaaatcttataactaaggctaatattttcataggtttgggatgttgcaagaaaccagttggttacagagatgaaggagcatgagaagcgtgtatggtccatcgattattcatcagcagatccaacattgttagctagtggtagtgatgatggttcggttaagctctggagtatcaatcaggcaaacttcgaaactaaaccaaaacgtatttatgctcggaaatgcgagaatagttcttttgagggatgtgtttttaacggttgagttcgttttcctctgtttcagggggttagtattggaaccatcaagacaaaggcaaacatatgttgtgttcagtttccatcagagacgggtcggtctttagcatttggctcagcagatcacaaagtttactactatgatctaaggaaccccaagcttcctctttgcacaatgattggtcaccacaagaccgtgagttatgttagatttgtagattcatcaacacttgtttcttcttcgacggataacacgctgaagctatgggacttatcaatgtctatttccggtattaatgaaacgcctcttcactcattcatgggacacactaatgttaaggtatatttttttcgagattcgtttctattgcttcatttcaagtttgctttatgagattacaaatacatttctgttcttgcattgtgtgtagaactttgttggattatcagtctctgacgggtatatagcaacaggctctgagactaacgaggtatgtctactctcacttcataaatatacaaaagaagaactgttgatcccatttgtcaataaaaaccgtgaatttcgtgtgcttcactggacaggtttttgtgtaccacaaggcatttccaatgccggttttgtcgtacaagttcaaaactatagaccctgtgtcggaactcgaagtagacgatgcctcgcagttcatatcctcagtctgctggcggggacagtcgtcgaccttagttgctgcaaactccaccggcaatatcaagattttggagatggtatgaaccaatataaagtggctcagaagcaaaatggtaatattccagggtttggattggtataggaaacaatttttgggagagaaagtaggaaaagtgaaaacaatattgttgctactaaagaattgtgtatatagaaaaaatatatgatatctttctatttgttgtatagtacttattgtaaaccctaatgacacaggtgggcaacttaacataca

SEQ ID NO: 8 - protein, 794 aa, Arabidopsis thaliana
MKGSSESSSRGLNNTSGVSEFCTDGSKSLSHIDYVRSLLGSHKEANLGGLDDDSIVRALECEDVSLRQWLDNPDRSVDAFECFHVFRQIVEIVNAAHSQGIVVHNVRPSCFVMSSFNNVSFIESASCSDSGSDEDATTKSREIGSSRQEEILSERRSKQQEEVKKQPFPMKQILAMEMSWYTSHEEDNGSLCNCASDIYRLGVLLFELFCPVSSREEKSRTMSSLRHRVLPPQILLNWPKEASFCLWLLHPEPSCRPSMSELLQSEFINEPRENLEEREAAMELRDRIEEQELLLEFLFLIQQRKQEAADKLQDTISLLSSDIDQVVKRQLVLQQKGRDVRSFLASRKRIRQGAETTAAEEENDDNSIDEESKLDDTLESTLLESSRLMRNLKKLESVYFATRYRQIKAATAAEKPLARYYSALSCNGRSSEKSSMSQPSKDPINDSRQGGWIDPFLEGLCKYLSFSKLRVKADLKQGDLLNSSNLVCAIGFDRDGEFFATAGVNKKIKIFECESIIKDGRDIHYPVVELASRSKLSGICWNSYIKSQVASSNFEGVVQVWDVARNQLVTEMKEHEKRVWSIDYSSADPTLLASGSDDGSVKLWSINQGVSIGTIKTKANICCVQFPSETGRSLAFGSADHKVYYYDLRNPKLPLCTMIGHHKTVSYVRFVDSSTLVSSSTDNTLKLWDLSMSISGINETPLHSFMGHTNVKNFVGLSVSDGYIATGSETNEVFVYHKAFPMPVLSYKFKTIDPVSELEVDDASQFISSVCWRGQSSTLVAANSTGNIKILEMV

SEQ ID NO: 9 - DNA, 1283 bp, Arabidopsis thaliana
gtcacaccaaaaggaagacgaatgacttggcggctagtgtaatagttttaaaaatgaccacataatctcaccagcctcaaaacctcacgacacgtcattctctccaattctacaaacaccattcatttcatttccctaaaaaattatggctcatgtaagtttctttgatgtgtgaactgtggaagagactactctcatcaaccatgaaccataaaaactccaccgctctttctctccctcaatcatttacatctcttccttaaatctctcttcccaccatcatcattccaaaccaattctctctcacttctttctggtgatcagagagatcgactcaatggtgagcttttgcgagctttgtggtgccgaagctgatctccattgtgccgcggactctgccttcctctgccgttcttgtgacgctaagttccatgcctcaaattttctcttcgctcgtcatttccggcgtgtcatctgcccaaattgcaaatctcttactcaaaatttcgtttctggtcctcttcttccttggcctccacgaacaacatgttgttcagaatcgtcgtcttcttcttgctgctcgtctcttgactgtgtctcaagctccgagctatcgtcaacgacgcgtgacgtaaacagagcgcgagggagggaaaacagagtgaatgccaaggccgttgcggttacggtggcggatggcatttttgtaaattggtgtggtaagttaggactaaacagggatttaacaaacgctgtcgtttcatatgcgtctttggctttggctgtggagacgaggccaagagcgacgaagagagtgttcttagcggcggcgttttggttcggcgttaagaacacgacgacgtggcagaatttaaagaaagtagaagatgtgactggagtttcagctgggatgattcgagcggttgaaagcaaattggcgcgtgcaatgacgcagcagcttagacggtggcgcgtggattcggaggaaggatgggctgaaaacgacaacgtttgagaaatattattgacatgggtcccgcattatgcaaattaggacatttagtgtttagtgccttaattatagtttgtgtcattgatctgtttgatctttcttttcaacgtaaactaaaaatggtctgaaccaattgagatgatagatatttctttagaggagatttaatttttttaatcaacagtaagaagattttagaaacggaataatggtaatccaatagtcattatcaggttcttttgagatgaaattgaaatcttttctttcccaa

SEQ ID NO: 10 - protein, 225 aa, Arabidopsis thaliana
MVSFCELCGAEADLHCAADSAFLCRSCDAKFHASNFLFARHFRRVICPNCKSLTQNFVSGPLLPWPPRTTCCSESSSSSCCSSLDCVSSSELSSTTRDVNRARGRENRVNAKAVAVTVADGIFVNWCGKLGLNRDLTNAVVSYASLALAVETRPRATKRVFLAAAFWFGVKNTTTWQNLKKVEDVTGVSAGMIRAVESKLARAMTQQLRRWRVDSEEGWAENDNV

SEQ ID NO: 11 - DNA, 5729 bp, Arabidopsis thaliana
accacacctcagcctcactcctccttcatctccgacgtcatacccaaaccactcttttctcacaattccgattttctcaaaaaccaaaatcacaatcgaagaaatcttttgaaagcaaaatggaagagatttcgacggatccggttgttccagcggtgaaacctgacccgagaacatcttcagttggtgaaggtgctaatcgtcatgaaaatgacgacggaggaagcggcggttctgagattggagcaccggatctggataaagacttgctttgtccgatttgtatgcagattattaaagatgctttcctcacggcttgtggtcatagtttctgctatatgtgtatcatcacacatcttaggaacaagagtgattgtccctgttgtagccaacacctcaccaataatcagctttaccctaatttcttgctcgataaggtttctttttctaattttactcgtaatttgtgtttggtccttagcttttcagttcaagttcgggtgttgcttgtttgagtcttagcttgtaatcaagtttggtttttgtcgtggtttttgtggaaacaagtaagctagcttagtctttaatcgattccaatgaaccttgccattagattttcttaggaaggttttgttttgtttttgatgtgtagtattgtggtaggattctgcttattacagctaaatagaagcatgtttataggaatgatagtgtattgaaactatttatcactggaatgcagcttattatatgacgtttatgtatcaattgtgtagctattgaagaaaacttcagctcggcatgtgtcaaaaactgcatcgcccttggatcagtttcgggaagcactacaaagggtcggttcttaacaagctttttatgtttcattttttcctctttaagtttgctttctcagaattattattggagtaaaaagcaattctaagtcatttcttttgatcaaatattgacttcctgcgttcatacttagacagggttgtgatgtgtcaattaaggaggttgataatcttctgacacttcttgcggaaaggaagagaaaaatggaacaggaagaagctgagaggaacatgcagatacttttggactttttgcattgtctaaggaagcaaaaagttgatgaactaaatgaggttggtttgtctgaattatggagtagcattgggctacctattgataattattctcgttgctcttcattccatttgcttcaaaatcttcactgggtttagactgtatagttgttgtaaaagaaagccataaaccatgtttctatcaatagatgtatgatctatccaactattcatgagacgtataacccctgaatgttagatgagatctcagaatattatagaattctgtcatttatgtcttctctatcttaatgtaggtgcaaactgatctccagtatattaaagaagatataaatgccgttgagagacatagaatagatttataccgagctagggacagatattctgtaaagttgcggatgctcggagatgatccaagcacaagaaatgcatggccacatgagaagaaccagattggtttcaactccaattctctcagcataagaggaggaaattttgtaggcaattatcaaaacaaaaaggtagaggggaaggcacaaggaagctctcatgggctaccaaagaaggatgcgctgagtgggtcagattcgcaaagtttgaatcagtcaactgtctcaatggctagaaagaaacggattcatgctcaggtttgttcctccttgtctactcaacatctttatgctgaaatggtaaaaggacaatggcattaaagtttaagatatttgtctattaaagaataagaataattactacaattaggccccagtgattgtgttacagttactaccatgcctagctactcagctcttggtcatctggattgacagctcgagaaattgaaagtggttgtgggtagaggatgaatcttcttacggaaaagataccttagattaaggcccacatttttggtggtctttataaaaagccgattggacttcagagagattgatttcgtattgtttgcacgtaaaagtagaaatagctttctggactttatatctactgcttagtgatagtagccgcttcttgtctctctggtactatagcaacaaaaagtcactatgttctctagacacactaacagttctgtgaatactatgcaatcacaattatagtagttttcttgaaaaaacctgcatggtcctttaatctctctgtaactgcttgttgatatatgcacagattgcctaattctgttaaagtgtcttgtcttgtagttcaatgatttacaagaatgttacctccaaaagcggcgtcagttggcagaccaaccaaatagtaaacaagaaaatgataagagtgtagtacggagggaaggctatagcaacggccttgcagattttcaatctgtgttgactaccttcactcgctacaggtagaggagtgactatcccttttcaatcggtctagccgtacataaagagtgttttcgaattagttggggttttaattgcactctttattcacagtcgtctaagagttatagcagaaatccggcatggggatatatttcattcagccaacattgtatcaaggtgaattttctattccttgttcgttgatcagataacagatgataggtaccaaaagaatgtctccgaatatatcaaaatagacccttaagtgattaagttttgtttaatgtattgatagacgcaattattactatgggattcgttgctgtgtaattctaagccagtgaatagtatgaagataaaactgctgacatggtagggagaaggttgtgtagtcagcaggccacttgaggagaattttctaaaagatggaacactatgttatccttatccatgtcggaatatatagaatcggtttgcgatcttttatatgaaagtgtctcttctcaatgaagagacgaaagatatatatttttagtttatggtctgggagctccggggctcatatctttttctctctatgtgctttggtttatggtcttccatagtttacatgctttatgctttgcctggacctctagtttacctgtaatttgatgtaccatcttactgattttgctgatgaagagggtgctgttccttttcagcatagagtttgatcgtgatgatgagctgtttgccactgctggtgtttctagatgtataaaggtttttgacttctcttcggtacgttcataaatcttggagtgcattcaattacataccaattgaatcttcttgtccaaccttatgcttttgtgaaactgatcatgcatctcaattctaagaaagcttcgttgataatgaaactaagttatgctcttgggaaattctagctttgatgcaagatagccattttacacttttgtattgcgtagaagaatataaatcaaagctaatcaaatttttggttttaattttctgttttatccaatggaccacgctgaactgattgattatcggctgctacttttcaggttgtaaatgaaccagcagatatgcagtgtccgattgtggagatgtcaactcggtctaaacttagttgcttgagttggaataagcatgaaaaaaatcacatagcaagcagtgattatgaaggaatagtaacagtgtgggatgtaactactaggcaggtgagtactttacccaaagtatctaacatcgcacctataagatattcatctcatgaattctgcagcgtatttgaatttttatgtaaactgaggcagagtcttatggagtatgaagagcacgaaaaacgtgcctggagtgttgacttttcacgaacagaaccatcaatgcttgtatctggtagtgacgactgcaaggtaaatatctccatattttccgttatcacctttcatctcacattgggctatgtctcatataaaaagaatactacttttagatatctcctagctcttataactaacttggtcacggatttgtctaatttctacgacgaactaaagagacatattgtgaaacaaaactggagaatctcctacttgctcattagtacattgtgttctcatgaatcttttctactacttcttaaacttttctttgagttgttcttcccaacttggggattggtcatatgtagcccttcctacattgaatgtttgttggtttcttaaattttcagcaaaattttccccttagtgtatctgtggtggctatacattaggaaacaaacgaaactggacacttgtatatagtagattttaccttgaaattttgtagcttcatttgtccaaatttaattaaacgttattgaaaaattcactggatttctaattctgacaaaagcatattataccagtgggctccttttaagtcaattcactactagttaatcctcaaccatgcattattgcatctgttaaatcatgtctcatagattaaccaattgtgctgtgtgtttgatccaggttaaagtttggtgcacgaggcaggaagcaagtgtgattaatattgatatgaaagcaaacatatgttgtgtcaagtacaatcctggctcaagcaactacattgcggtatatacttccacgcctcaactttgcatctcacatttattcgctaccactcatgatcagcatgacactatcacacgtctctagatataatctttaattagatagagagacttagacgtttaagcctgttgacttatcacaggtcggatcagctgatcatcacatccattattacgatctaagaaacataagccaaccacttcatgtcttcagtggacacaagaaagcagtttcctatgttaaatttttgtccaacaacgagctcgcttctgcgtccacagatagcacactacgcttatgggatgtcaaagacaacttgccagttagattactgcccgagccaatcttgcagattcagtcaaaaagaaaaacttgtcttgactatgttgttcaattttacttttcaggttcgaacattcagaggacatactaacgagaagaactttgtgggtctcacagtgaacagcgagtatctcgcctgtggaagcgagacaaacgaagtatatgtatatcacaaggttcacaaatccgaaacctctgctctcaactttagcatttttcattcatgattcacagattttcaacctgtcgttgctcaagtattttaaaatgtgaatgatgcgatgcaggaaatcacgagacccgtgacatcgcacagatttggatcgccagacatggacgatgcagaggaagaggcaggttcctactttattagtgcggtttgctggaagagtgatagtcccacgatgttgactgcgaatagtcaaggaaccatcaaagttctggtactcgctgcgtgattctagtagacattacaaaagatcttatagcttcgtgaatcaataaaaacaaatttgccgtctatgttctttagtgggagttacatatagagagagaacaatttattaaaagtagggttcatcatttggaaagcaactttgtattattatgcttgccttggaacactcctcaagaagaatttgtatcagtgatgtagatatgtcttacggtttcttagcttctactttatataattaaatgttagaatcaaaatgcggtgatgtgaatcggtcatgaaaagtcaaaccttggcttatacaaaaggtccgacccatgcaataagtcat

SEQ ID NO: 12 - protein, 675 aa, Arabidopsis thaliana
MEEISTDPVVPAVKPDPRTSSVGEGANRHENDDGGSGGSEIGAPDLDKDLLCPICMQIIKDAFLTACGHSFCYMCIITHLRNKSDCPCCSQHLTNNQLYPNFLLDKLLKKTSARHVSKTASPLDQFREALQRGCDVSIKEVDNLLTLLAERKRKMEQEEAERNMQILLDFLHCLRKQKVDELNEVQTDLQYIKEDINAVERHRIDLYRARDRYSVKLRMLGDDPSTRNAWPHEKNQIGFNSNSLSIRGGNFVGNYQNKKVEGKAQGSSHGLPKKDALSGSDSQSLNQSTVSMARKKRIHAQFNDLQECYLQKRRQLADQPNSKQENDKSVVRREGYSNGLADFQSVLTTFTRYSRLRVIAEIRHGDIFHSANIVSSIEFDRDDELFATAGVSRCIKVFDFSSVVNEPADMQCPIVEMSTRSKLSCLSWNKHEKNHIASSDYEGIVTVWDVTTRQSLMEYEEHEKRAWSVDFSRTEPSMLVSGSDDCKVKVWCTRQEASVINIDMKANICCVKYNPGSSNYIAVGSADHHIHYYDLRNISQPLHVFSGHKKAVSYVKFLSNNELASASTDSTLRLWDVKDNLPVRTFRGHTNEKNFVGLTVNSEYLACGSETNEVYVYHKEITRPVTSHRFGSPDMDDAEEEAGSYFISAVCWKSDSPTMLTANSQGTIKVLVLAA

SEQ ID NO: 13 - DNA, 3618 bp, Arabidopsis thaliana
aataaaagttgtggtggaaaattattccaataaatctcgtcgtgtgaattgaatcattattcctagtgtcaatcttcttcacctctctctatcaacctccaatttcttcttcttctttttttttagataacttatttttattttatttttctttgatttttcactttaagacaaaaataataattttatatttctaccaaaaataaataataattataatttaataagagataaatattttttcaaactgtaacctaaacaataaataaattaaaagagaaattaatgcaaatattgctatatttttgggttattacttagagactactttgtccaaaattaggaaaaatctttttgggtgaataattcccaaaagtatagttgcattttgccagagaggcactcagagagagagagagatattcgtaaagataagctttttttttttttttgccttctcatcaaaaatctttttttttttgttgtctttcttctcgagagagataagtgaccaaagggtttcgatttctggaaatagtttgaataaaaaagtaatttttatgtgattattggcaaagaaaagttttagttttttttagtttgtgagagatgtctggttctgtatctggttgtggttctggtggttgtagtattgtatggtttagaagagatcttagggttgaagataatccagctttagcagcagcagtaagagctggtccagtgattgctctgtttgtttgggcaccagaagaagaaggacactatcatccaggtagggtttctaggtggtggctcaagaacagtttggctcagcttgattcttctcttagaagtcttggtacttgtcttatcaccaagagatctactgatagtgttgcttctcttcttgatgttgttaaatccactggtgcttctcagatcttcttcaaccatttgtatggtctgttgttcctcttctcttctctctttatcttatctttgaatttgcatgataaaaagatttgatttttgtgattggaatcatcttttctcttatgggttatgttttgtttgatgtatttgtggaataagatgatctggaaatgataaagttttgatctttttcagatccattgtctttggtgcgtgatcaccgagctaaagatgttttgacggcgcaaggcatagcggttcgatcattcaacgcagacttgctttatgagccatgggaagtgactgatgaattaggccgtcctttctctatgtttgctgcgttttgggagagatgtcttagtatgccttatgaccctgagtctcctcttcttccacctaagaagatcatttcaggtttgtttctttgccactctctcttgtgggttttcgttttttccaaatctgtatcattcaataatgtttcttttatgttgcaggggatgtgtctaaatgtgttgcggatccattggtgtttgaggatgactctgagaaaggaagcaatgcacttctggctcgtgcttggtctcctggatggagtaatggtgataaagctctcacaacgtttataaacggtccattgcttgaatactctaagaaccgcagaaaagccgatagtgctacaacctcgtttctttctccacacttgcattttggggaagtgagtgtgagaaaagtttttcatcttgttcggatcaaacaggtcgcgtgggcaaacgaaggaaacgaggccggggaagaaagcgtgaatcttttcctgaaatctattggtctcagggagtattctaggtacataagttttaaccatccatattcccatgaaagaccacttcttggccatctaaagttcttcccttgggctgtggatgagaactatttcaaggcatggaggcaaggccggactggatatccgttggtcgatgccgggatgagagagttatgggctactggttggttgcatgatcgcataagagtagttgtttcaagcttctttgttaaagtgcttcaattaccatggagatgggggatgaagtatttctgggacacacttcttgatgcggatttagaaagcgatgctcttggttggcaatacattaccggtactctcccggatagccgggagtttgatcgcatagataaccctcagtttgaagggtacaagtttgatccaaatggtgaatacgtaaggcgatggcttcctgaactctctagactcccgacagactggatacatcatccgtggaacgcacctgagtccgttcttcaagctgctggtatcgagcttggatcaaactatcctctaccaattgtaggattagacgaagcaaaagcacggcttcatgaagcgctttcacagatgtggcaactagaagctgcttcaagagctgcaatagagaacggatccgaagaaggacttggagattctgctgaggtagaggaagctcctatagagttcccaagggacattacaatggaagagactgaaccaaccagactcaacccaaacaggagatatgaggatcagatggttccaagcattacttcttctttgatcagacctgaagaagacgaagagtcgtctcttaatttgagaaattcagtaggagatagcagagcagaggttccaaggaacatggttaacaccaaccaagctcagcagcggagagcagaaccggcttcaaaccaagtcactgctatgattccagaatttaatatcagaattgttgcagagagcactgaagactcaacagcggaatcttccagcagcggaaggagagaaagaagcggaggcatagtccccgagtggtctccagggtactcagagcagttccctagtgaagaaaatggtattggaggaggaagtacaacgtctagctacttgcagaatcaccatgaaatactgaactggagacggctttcacaaaccgggtatgtaactcgcaaatccgactctctcaacaataagttcacataaagattctaaacattttcaaattgaaagtctccaaattttcagtaagtttcaatcttcctacacaattcaaataaatcgtcgatgcgcgttgtgcttggcacacttgtctagccaagtagtatcagttttaaattgaatttagctgaaaagttttgaactgttttcaaatccaagtgtttgcatttacagtagaccacagtaaaaaaattagtgaaagtttctatgtttcacagttcaattggaatttggtacattggtctaaccaagtggcatgaaatttactgtggtatgtttggttttacaggtaaaaagtgcatttggaggtgcaaaaggagaggaacatcataagggctgtaactccggtgaaatctggttggactgtaaaccgagtacatttggtacggtttaatgtaattccggttatggggctggagagaaactatgtaggagtttgtctgatgtacattttttatttatctctggttccatcatgttataatacactgtatagtaagtagtctgttgcttgtggtattagaccaggtctcatacttgttggctttcaaagttttggttataagtcaaacagatgccaattcttaatcttataaacatatatagttcacatttatttatgataaatgacttgttagtgtatatgaaacacgtac

SEQ ID NO: 14 - protein, 681 aa, Arabidopsis thaliana
MSGSVSGCGSGGCSIVWFRRDLRVEDNPALAAAVRAGPVIALFVWAPEEEGHYHPGRVSRWWLKNSLAQLDSSLRSLGTCLITKRSTDSVASLLDVVKSTGASQIFFNHLYDPLSLVRDHRAKDVLTAQGIAVRSFNADLLYEPWEVTDELGRPFSMFAAFWERCLSMPYDPESPLLPPKKIISGDVSKCVADPLVFEDDSEKGSNALLARAWSPGWSNGDKALTTFINGPLLEYSKNRRKADSATTSFLSPHLHFGEVSVRKVFHLVRIKQVAWANEGNEAGEESVNLFLKSIGLREYSRYISFNHPYSHERPLLGHLKFFPWAVDENYFKAWRQGRTGYPLVDAGMRELWATGWLHDRIRVVVSSFFVKVLQLPWRWGMKYFWDTLLDADLESDALGWQYITGTLPDSREFDRIDNPQFEGYKFDPNGEYVRRWLPELSRLPTDWIHHPWNAPESVLQAAGIELGSNYPLPIVGLDEAKARLHEALSQMWQLEAASRAAIENGSEEGLGDSAEVEEAPIEFPRDITMEETEPTRLNPNRRYEDQMVPSITSSLIRPEEDEESSLNLRNSVGDSRAEVPRNMVNTNQAQQRRAEPASNQVTAMIPEFNIRIVAESTEDSTAESSSSGRRERSGGIVPEWSPGYSEQFPSEENGIGGGSTTSSYLQNHHEILNWRRLSQTG

SEQ ID NO: 15 - DNA, 1739 bp, Arabidopsis thaliana
atctcaattgcctttggatccttgtatttcctcaaggctcacctttctccacgattcactctcgatatccgttcgattcttcagagatctgacggcggtagccagagtaatctattccttcccaaaatgtctcgcaattagattctttccaagttcttctgtaaatcccaagtcccgctcttttcctctttatccttttcaccagcttcgctactaagacaacaaatctttccctctctctctcgcctgatcgatcttcaaagagtaagaaaaatgcaggaacaagcgactagctctttagctgcaagctctttaccatcaagcagcgagaggtcatcaagctctgctccacatttggagatcaaagaaggtaaacaaaaattgtcgattgttttattttcatatcttctcccaacgagtgatctcattgattttcattctcatgcattggatatctagtaaattcacatctagattttgtatcaccattgattcacatctagattcacaacttttagtctcccccagaagatctgcaattttttattgtatttaaaaagtttgaaacttttcgtctaaagtctcttttatgttttaaaggaattgaaagcgatgaggagatacggcgagtgccggagtttggaggagaagctgtcggaaaagaaacttccggtagagaatctggatcggcgaccggtcaggagcggacacaggcgactgtcggagaaagtcaaaggaagcgagggaggacaccggcggagaaagagaacaagcggctgaagaggtgtctttttctctattcattgaatttacatttttaccctttatatggaaggtgattatgttaaataacggttgcgtgatggggttgtttggtaattttgtggtgaaaaggttgttgaggaacagagtttcagctcagcaagcaagagagaggaaaaaggcttacttgagcgagttggaaaacagagtgaaagacttggagaacaaaaactctgaacttgaagagcgactctctactcttcagaacgagaaccagatgcttagacatgtacgttttgtcgatcttactgttgacttaacctgcaacgtcaatgcttttgccatttgttttttgatatatcacagtactggtttatcacatttacgagtctaggattgaccaataattagtttagttagttaaaaggatctaagaagctaacttaagctttagattaattctcagaaggcatgtaggttcttgaaatggtcattcactcgcatattctctcaaaaggcatgtaaattcttcaatggttcttgaaatgctcactcattttgttctatatgaatggagaagatgagattgcaaagttggatctctatgtgtgtgtgtggtaataaagtcttttgagttgttatgttttttatgtggtgtagattctgaagaacacaacaggaaacaagagaggaggtggtggtggttctaatgctgatgcaagcctttgatctccttcttcttcttgtgttatatttttgtggataaaatttacagagaattgtatcaataattatcatgttaaaattatatgggatgtgagagctaatattgcaattgtagaccaagttctcttattgtagtcttagatttctcttaattgaaacataatgttgttttataacaaaaataagctaatttttgttctatgatatgacattgtgacactgtagtagaaaggttgcgttttcgattcccg

SEQ ID NO: 16 - protein, 168 aa, Arabidopsis thaliana
MQEQATSSLAASSLPSSSERSSSSAPHLEIKEGIESDEEIRRVPEFGGEAVGKETSGRESGSATGQERTQATVGESQRKRGRTPAEKENKRLKRLLRNRVSAQQARERKKAYLSELENRVKDLENKNSELEERLSTLQNENQMLRHILKNTTGNKRGGGGGSNADASL

SEQ ID NO: 17 - DNA, 2981 bp, Arabidopsis thaliana
gtcaagttaaagataattttggtatatatgagaaaggtatcgacaaaaaccataacgctatagatgattgtgatttgacaaaaacaccctcaaatcattgttttcagagtttttttagataaggtacagataagaaaccacctctaaaaatcaagcaatagatctcatcgcttaaaagaagagagagatcttcacttgtatgtgtcccactgattccaacacaatgtcccagaacttgccacgtgtcgttcatttcaaaagattgcagtactgttgtccctagagaatcattatctccctcgctgtaatatctttatgctcctgtcactttctgtctgtacccaaaagaagtaatgaacctctctcatcttcttcttctctgtttctttcatgttttgtgagttgtttctcaacaattttctggtctcttagagtgagaggagagagatagagagttgtgttgggcgtggaacttggactagttccacatatcaggttatatagatcttctctttcaacttctgattcgtccagaagctttcctaatctggtcagtagtactctttttatacgggtttttggttttataagatgtggctatatttggaaataactattttgcaagctttcctagattgccagaatataaaaaaagatgtttaacaagagaacggactcatggacttgctttaaattttaattattttaaaatcattctataatgattagagtaaataaactattaggactctgaattataaaattcgattttatatatgctcctccttgtatctcttaatcataagttatcattagctctgttcactagtgctataaaatatattctgaggttaataaaacttttttttctttttattttgaaatgtctccagagatctgacatggaacaccaaggttggagttttgaggagaattatagtttgtccactaatagaagatctatcaggtattatttaataatctttgatctaatgttttcattcattaaaaaaaaaactttataatatagtctgtgtgtgtgtgtattgatgtctaggccacaagatgaactagtggagttattatggcgagatggacaagtggttctgcagagccaaactcatagagaacaaacccaaacccagaaacaagatcatcatgaagaagccctaagatccagcacctttcttgaagatcaagaaactgtctcttggatccaataccctccagatgaagacccattcgaacccgacgacttctcctcccacttcttctcaaccatggatcccctccagagaccaacctcagagacggttaagcctaagtccagtcctgaacctcctcaagtcatggttaagcctaaggcctgtcctgaccctcctcctcaagtcatgcctcctccaaaatttaggttaacaaattcatcatcggggattagggaaacagaaatggaacagtactcggtaacgaccgttggacctagccattgcggaagcaacccatcacagaacgatctcgatgtctcaatgagtcatgatcgaagcaaaaacatagaagaaaagcttaatccgaacgcaagttcctcatcaggtggctcctctggttgcagctttggcaaagatatcaaagaaatggctagtggaagatgcatcacaaccgaccgtaagagaaaacgtataaatcacactgacgaatctgtatctctatcagatgtatgcttcctttaattctaactcatgataattacttcttgaaaacgtaatcttggtctatctcagagagtaatacataatacattttgcaggcaatcggtaacaagtcgaaccaacgatcaggatcaaaccgaaggagtcgagcagctgaagttcataatctctccgaaagggtacttcatcggttcgtctatataatatataatatatagtttaatgttagagaatcgtataacatttatacaaagaaaacagatacaaaactctgttttttattttcttcttttcataacttaaatagaggaggagagataggatcaatgagagaatgaaggctttgcaagaactaatacctcactgcagtaaagtgagttgatacaaatcaaatctaaaagtgaatcagattaagaaatgaagattgattcattcattggtgtgtttttgcagactgataaagcttcgattttagacgaagccatagattatttgaaatcacttcagttacagcttcaagtgatgtggatggggagtggaatggcggcggcggcggcttcggctccgatgatgttccccggagttcaacctcagcagttcatacgtcagatacagagcccggtacagttacctcgatttccggttatggatcagtctgcaattcagaacaatcccggtttagtttgccaaaacccggtacaaaaccagatcatctccgaccggtttgctagatacatcggtgggttcccacacatgcaggccgcgactcaggtaaggccaatacgaagccgtgtttattaaacgacgattttggtaaaatgttttttgggtttactacattaatacgagataagaaaaaatgttaaaccgacgtagttttagttgacacgttgtgtggttttttattagatgcagccgatggagatgttgagatttagttcaccggcgggacagcaaagtcaacaaccgtcgtctgtgccgacgaagaccaccgacggttctcgtttggaccactaggttggtgagccacttttttacttccttatttttggtatgtttcttttttatatctatctttctgaacatacttaaaacgttcaaggatgtattattatagagtaaacgtgcaacttcattacgttattttctgtatatgtgagtttatgtatgtcaaaatgacatgatgagattttttgtaaacaacatcttaaaaacaggacatgtgatttttgtaatcgtaaaaactttgggatgcagtttattttctaatcaaaa

SEQ ID NO: 18 - protein, 430 aa, Arabidopsis thaliana
MEHQGWSFEENYSLSTNRRSIRPQDELVELLWRDGQVVLQSQTHREQTQTQKQDHHEEALRSSTFLEDQETVSWIQYPPDEDPFEPDDFSSHFFSTMDPLQRPTSETVKPKSSPEPPQVMVKPKACPDPPPQVMPPPKFRLTNSSSGIRETEMEQYSVTTVGPSHCGSNPSQNDLDVSMSHDRSKNIEEKLNPNASSSSGGSSGCSFGKDIKEMASGRCITTDRKRKRINHTDESVSLSDAIGNKSNQRSGSNRRSRAAEVHNLSERRRRDRINERMKALQELIPHCSKTDKASILDEAIDYLKSLQLQLQVMWMGSGMAAAAASAPMMFPGVQPQQFIRQIQSPVQLPRFPVMDQSAIQNNPGLVCQNPVQNQIISDRFARYIGGFPHMQAATQMQPMEMLRFSSPAGQQSQQPSSVPTKTTDGSRLDH

SEQ ID NO: 19 - DNA, 4699 bp, Arabidopsis thaliana
aaaaaagtcgcagaaaatatatgaggaaacaaaaagcgaagacgacaaaaaaaaaaaaaactctgatttttttttgttatctctctctatctgagaggcacacattttgcttcgtcttcttcaatttattttattggtttctccacttatctccgatctcaattctccccattttcttcttcctcaagttcaaaattcttgagaatttagctctaccagaattcgtctccgataactagtggatgatgattcaccctaaatccttccttgtctcaaggtaattctgagaaatttctcaaattcaaaatcaaacggcatggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgcagttgcaagagagatggttaggctcattgagactgcaactgttcctatattcgctgtggatgccggaggctgcatcaatggatggaacgctaagattgcagagttgacaggtctctcagttgaagaagctatggggaagtctctggtttctgatttaatatacaaagagaatgaagcaactgtcaataagcttctttctcgtgctttgagaggtatattcagttcttcagctatgttgtatctgcggtgtatataccaattcgcgggtatttgattattttgttgcatttggcaatgcaggggacgaggaaaagaatgtggaggttaagctgaaaactttcagccccgaactacaagggaaagcagtttttgtggttgtgaatgcttgttccagcaaggactacttgaacaacattgtcggcgtttgttttgttggacaagacgttactagtcagaaaatcgtaatggataagttcatcaacatacaaggagattacaaggctattgtacatagcccaaaccctctaatcccgccaatttttgctgctgacgagaacacgtgctgcctggaatggaacatggcgatggaaaagcttacgggttggtctcgcagtgaagtgattgggaaaatgattgtcggggaagtgtttgggagctgttgcatgctaaagggtcctgatgctttaaccaagttcatgattgtattgcataatgcgattggtggccaagatacggataagttccctttcccattctttgaccgcaatgggaagtttgttcaggctctattgactgcaaacaagcgggttagcctcgagggaaaggttattggggctttctgtttcttgcaaatcccgagccctgagctgcagcaagctttagcagtccaacggaggcaggacacagagtgtttcacgaaggcaaaagagttggcttatatttgtcaggtgataaagaatcctttgagcggtatgcgtttcgcaaactcattgttggaggccacagacttgaacgaggaccagaagcagttacttgaaacaagtgtttcttgcgagaaacagatctcaaggatcgtcggggacatggatcttgaaagcattgaagacgggtgagtatagttagaatttatctagaagctagttttgcttacttcacaaaatgtgaccaaatcccaaattttgtttttttcattgatcagttcatttgtgctaaagagggaagagtttttccttggaagtgtcataaacgcgattgtaagtcaagcgatgttcttattaagggacagaggtcttcagctgatccgtgacattcccgaagagatcaaatcaatagaggtttttggagaccagataaggattcaacagctcctggctgagtttctgctgagtataatccggtatgcaccatctcaagagtgggtggagatccatttaagccaactttcaaagcaaatggctgatggattcgccgccatccgcacagaattcaggtacatttcattgttcccgctgttgtctccacatatccataaccaaaattatgcaatccggtttttttggttccttattttgtacataaagaaaatgaatttggtttggttaattacgaatttgatttaggcgtttaaagaatttgaggttttaaccaattcactatttgttttggttattgtttagttggaacctagattagtttgatttttgtattcggtttagtcgacttgggaacttttagacacatccataggcctagaattagcagtcaaggaatgtaatgttttcaaattgatgaaaaccagctcaaaagtgtaaaacttgggtttcatgtgttggtgtctttgttatgtctttattcgttgtttgcagaatggcgtgtccaggtgaaggtctgcctccagagctagtccgagacatgttccatagcagcaggtggacaagccctgaaggtttaggtctaagcgtatgtcgaaagattttaaagctaatgaacggtgaggttcaatacatccgagaatcagaacggtcctatttcctcatcattctggaactccctgtacctcgaaagcgaccattgtcaactgctagtggaagtggtgacatgatgctgatgatgccatattagtcacacttcagttggtatgagagtttgtatcattgtatgagtgtttgtgtgtctaacgacgtcggaggaggatagaaagttttttttttgtttccggtgagattagtagagaagagggagattatttgcgttcagctcagctcgccggaaaaaaaacgtaacagtagttgtagagaatttcaagacttttgtttgtgctgtgtaaattgacaactccgagagaaacaaaacaatgagataagaagagagcatattaatcgatgaccaatccttttaatttgttactcgttacatcaattcaaaattatta

SEQ ID NO: 20 - protein, 1172 aa, Arabidopsis thaliana
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAVQRRQDTECFTKAKELAYICQVIKNPLSGMRFANSLLEATDLNEDQKQLLETSVSCEKQISRIVGDMDLESIEDGSFVLKREEFFLGSVINAIVSQAMFLLRDRGLQLIRDIPEEIKSIEVFGDQIRIQQLLAEFLLSIIRYAPSQEWVEIHLSQLSKQMADGFAAIRTEFRMACPGEGLPPELVRDMFHSSRWTSPEGLGLSVCRKILKLMNGEVQYIRESERSYFLIILELPVPRKRPLSTASGSGDMMLMMPY

SEQ ID NO: 21 - protein, 1019 aa, Solanum lycopersicum
MDKSKEEADANDVGANVLSRTRECDSGEPSMGKCTSTSHELPEGSTSASSGMLESDGMNRNVTSMKGPQLHGTSSYSLNSSRLTLEKLCSYKISEPASLRCSNSQKTIQKPQIQWQRFYQLGSGSRSLKGDGDPSSTDKAVQQLSSKELPRINLLALKMLKQASDKDIKGGSNAESSQSTEDHNLIIPRNRLLPGSSQSKLLSTSSFSHFFANRSLKTKDVLPKGPALHKEVHIASTLQNKNDFEQASTGMVSSDVFLKQGANSNQASFSRSDHQRPTSTYNGFTLREWLNSTGSQINKAERIHIFRQIVKLIDIAHSEGNAFQDIRPSCFILLSPNGVKYIGSSVQIDSMYAVSRNTNGKRPSDMEMHANSNLGIKLQKVNVDVGLIRQQPESNARSCSRDEGTSFQAGCLLESDINQLEKKWYTCPEELHHESLASSNIYSLGVLFFELLCCFESPAAHSTAMLNLQSRILPPNFLCQNPKEVGFCFLLLHPVPSSRPTTREILQSELIIGAEEVCKIDGVPSFIEKDDDPDSDVLLYFLVSLQEEKQNDTSKLLQRIECLEADIKDVEKREVLRHSDWVETDFNNMRQGSYLKHLNSADSISRSFSIPNMRNEKLMKNISQLESAYFCMRSQIQLAENDTIGRTDTDLLTSRDRLFQVSAKEAEPILKSVDRVGAFFEGICKYARYCKFEEYGTLRNGDLLNSINVICSLCFDYEEDYIAAAGVSKKIKIFEFASLLNESADLQYPVAEMSNISKLSCVSWNKYMRNYLASTDYDGVVKMWDASTGQEFSQHTEHQKRAWSVDFSQVNPTKFATGSDDCSVKVWNINERSSVDTIWNPANICCVQFSAYSSHLLAFGSADYKIYCYDLRHTRIPWCTLTGHEKAVSFVKFLDYGTLVSASTDNTLKLWDLKRTSLEGLSSNACSLTFKGHTNEKNFVGLSVLDGYIACGSESNEVYAYHRSLPMPITSYKFGSVDPSSGNDGESNGKFVSSVCWRRKSNMVVAANSTGCIKLLRLV

SEQ ID NO: 22 - protein, 964 aa, Solanum lycopersicum
MDKSKEEADANDVGANVLSRTRECDSGEPSMGKCTSTSHELPEGSTSASSGMLESDGMNRNVTSMKGPQLHGTSSYSLNSSRLTLEKLCSYKISEPASLRCSNSQKTIQKPQIQWQRFYQLGSGSRSLKGDGDPSSTDKAVQQLSSKELPRINLLALKMLKQASDKDIKGGSNAESSQSTEDHNLIIPRNRLLPGSSQSKLLSTSSFSHFFANRSLKTKDVLPKGPALHKEVHIASTLQNKNDFEQASTGMVSSDVFLKQGANSNQASFSRSDHQRPTSTYNGFTLREWLNSTGSQINKAERIHIFRQIVKLIDIAHSEGNAFQDIRPSCFILLSPNGVKYIGSSVQIDSMYAVSRNTNGKRPSDMEMHANSNLGIKLQKVNVDVGLIRQQPESNARSCSRDEGTSFQAGCLLESDINQLEKKWYTCPEELHHESLASSNIYSLGVLFFELLCCFESPAAHSTAMLNLQSRILPPNFLCQNPKEVGFCFLLLHPVPSSRPTTREILQSELIIGAEEVCKIDGVPSFIEKDDDPDSDVLLYFLVSLQEEKQNDTSKLLQRIECLEADIKDVEKREVLRHSDWVETDFNNMRQGSYLKHLNSADSISRSFSIPNMRNEKLMKNISQLESAYFCMRSQIQLAENDTIGRTDTDLLTSRDRLFQVSAKEAEPILKSVDRVGAFFEGICKYARYCKFEEYGTLRNGDLLNSINVICSLCFDYEEDYIAAAGVSKKIKIFEFASLLNESADLQYPVAEMSNISKLSCVSWNKYMRNYLASTDYDGVVKMWDASTGQEFSQHTEHQKRAWSVDFSQVNPTKFATGSDDCSVKVWNINERSSVDTIWNPANICCVQFSAYSSHLLAFGSADYKIYCYDLRHTRIPWCTLTGHEKAVSFVKFLDYGTLVSASTDNTLKLWDLKRTSLEGLSSNACSLTFKGHTNEKNFVGLSVLDGYIACGSESNECLLEKKI